(19) United States
(12) Patent Application Publication
(10) Pub. No.: US 2010/0209997 A1
(43) Pub. Date: Aug. 19, 2010

(54) CARBONIC ANHYDRASE POLYPEPTIDES AND USES THEREOF

(75) Inventors: Lisa M. Newman, San Jose, CA (US); Louis Clark, San Francisco, CA (US); Charlene Ching, San Jose, CA (US); Sabrina Zimmerman, Woodside, CA (US)

Correspondence Address: Codexis, Inc., 200 Penobscot Drive, Redwood City, CA, 94063, US

(73) Assignee: CODEXIS, INC., Redwood City, CA (US)

(21) Appl. No.: 12/684,800

(22) Filed: Jan. 8, 2010

Related U.S. Application Data
(60) Provisional application No. 61/143,734, filed on Jan. 9, 2009.
(60) Provisional application No. 61/144,111, filed on Jan. 12, 2009.
(60) Provisional application No. 61/247,315, filed on Sep. 30, 2009.

(51) Int. Cl. A62D 3/02; C07H 21/04; C12N 9/88; C12N 15/63; C12N 1/21; C12N 1/19; C12N 1/15

(52) U.S. Cl. 435/232; 536/23.2; 435/320.1; 435/266; 435/252.33; 435/254.21; 435/252.31; 435/254.3; 435/254.6; 435/254.11; 435/254.7; 435/254.5; 435/262.5

(57) ABSTRACT

The present disclosure relates to recombinant carbonic anhydrase enzymes having improved properties as compared to a naturally-occurring wild type carbonic anhydrase and uses thereof for the sequestration of carbon dioxide as well as for the release of carbon dioxide from a composition comprising bicarbonate. Also provided are polynucleotides encoding the recombinant carbonic anhydrase enzymes and host cells capable of expressing the recombinant carbonic anhydrase enzymes.

### CROSS REFERENCE TO RELATED APPLICATIONS

The present application claims priority of U.S. provisional patent applications 61/143,734, filed Jan. 9, 2009, 61/144,111, filed Jan. 12, 2009, and 61/247,315, filed Sep. 30, 2009, each of which is hereby incorporated by reference herein.

### TECHNICAL FIELD

The present disclosure relates to carbonic anhydrase polypeptides and uses thereof. The present disclosure further relates to nucleic acids encoding carbonic anhydrase polypeptides, expression systems for the production of carbonic anhydrase polypeptides, as well as to methods and bioreactors for the capture and sequestration of carbon dioxide using the carbonic anhydrase polypeptides of the present disclosure.

### REFERENCE TO SEQUENCE LISTING, TABLE OR COMPUTER PROGRAM

The Sequence Listing concurrently submitted electronically under 37 C.F.R. §1.821 via EFS-Web in a computer readable form (CRF) as file name CX3-009US1_ST25.txt is herein incorporated by reference. The electronic copy of the Sequence Listing was created on Jan. 8, 2010 with a file size of 460 Kbytes.

### BACKGROUND

The enzyme, carbonic anhydrase (“CA”) (EC 4.2.1.1), catalyzes the reversible reactions depicted in Scheme 1:

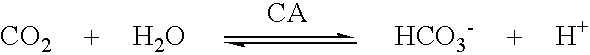

In the forward or “hydration” reaction, CA combines carbon dioxide and water to provide bicarbonate and a proton, or depending on the pH, to provide carbonate (CO₃⁻²) and two protons. In the reverse, or “dehydration” reaction, CA combines bicarbonate and a proton to provide carbon dioxide and water. Carbonic anhydrases are metalloenzymes that typically have Zn⁺² in the active site. However carbonic anhydrases having e.g. Co⁺² or Cd⁺² in the active site have been reported. At least three classes of carbonic anhydrases have been identified in nature.

The α-class carbonic anhydrases are found in vertebrates, bacteria, algae, and the cytoplasm of green plants. Vertebrate α-carbonic anhydrases are among the fastest enzymes known, exhibiting a turnover number (k_{cat}) (the number of molecules of substrate converted by an enzyme to product per catalytic site per unit of time) of 10⁶ sec⁻¹. The β-class carbonic anhydrases are found in bacteria, algae, and chloroplasts, while γ-class carbonic anhydrases are found in Archaea and some bacteria. Although carbonic anhydrases of each of these classes have similar active sites, they do not exhibit significant overall amino acid sequence homology and they are structurally distinguishable from one another. Hence, these three classes of carbonic anhydrase provide an example of convergent evolution.

It has been suggested that carbonic anhydrase could be used as a biological catalyst to accelerate the capture of carbon dioxide produced by produced by combustion of fossil fuels. However, the carbonic anhydrases found in nature are not ideally suited for use in such applications. Accordingly, there is a need in the art for engineered carbonic anhydrases that can effectively hydrate carbon dioxide at elevated temperatures and at alkaline pH for extended periods of time in the presence of relatively high concentrations of carbonate. In addition, such carbonic anhydrases should also be stable to variations in pH, e.g. stable not only at a relatively alkaline pH suitable for hydration and sequestration of carbon dioxide but also at a relatively acidic pH suitable for subsequent release and/or recapture of the hydrated and/or sequestered carbon dioxide.

### SUMMARY

The present disclosure provides heat-stable carbonic anhydrases that are capable of catalyzing the hydration of carbon dioxide at elevated temperatures. The present disclosure also provides carbonic anhydrases that are capable of catalyzing the hydration of carbon dioxide in the presence of relatively high concentrations of carbonate. In particular, the present disclosure provides heat-stable carbonic anhydrases that are capable of catalyzing the hydration of carbon dioxide at elevated temperatures in the presence of relatively high concentrations of carbonate.

The present disclosure also provides polynucleotides encoding the carbonic anhydrase enzymes of the disclosure, methods and hosts cells for the expression of those polypeptides, as well as methods and bioreactors for using the presently disclosed polypeptides.

In one aspect, the carbonic anhydrase polypeptides described herein have an amino acid sequence that has one or more amino acid differences as compared to a wild-type carbonic anhydrase or an engineered carbonic anhydrase that result in an improved property of the enzyme. Generally, the engineered carbonic anhydrase polypeptides have an improved property as compared to the naturally-occurring wild-type carbonic anhydrase enzymes obtained from Methanosarcina thermophila (“M. thermophila”; SEQ ID NO: 2). Improvements in an enzyme property include increases in thermostability, solvent stability, increased level of expression, enzyme activity at elevated pH, and enzyme stability and/or activity during pH variations, as well as reduced product inhibition (e.g., product inhibition by carbonate or bicarbonate). Improvements in an enzyme property of engineered carbonic anhydrases disclosed herein also include increased stability, solubility, and/or activity in the presence of additional reagents useful for absorption or sequestration of carbon dioxide, including, for example, calcium ions, aqueous carbonate solutions, amines such as monoethanolamine (MEA), methyldiethanolamine (MDEA), 2-aminomethylpropanolamine (AMP), 2-(2-aminoethylamino)ethanol (AEE), triethanolamine, 2-amino-2-hydroxymethyl-1,3-propanediol (Tris), piperazine, piperazine mono- and diethanolamine, ammonia, and mixtures thereof.

In certain embodiments, the present disclosure provides a recombinant carbonic anhydrase polypeptide having an improved enzyme property relative to the reference sequence of SEQ ID NO:2, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO:2, and at least one of the following amino acid substitutions at the position corresponding to the indicated position of SEQ ID NO:2: residue at position 2 is an aliphatic or non-polar amino acid selected from the group consisting of alanine, leucine, isoleucine, valine, glycine, and methionine, or a polar amino acid selected from the group consisting of asparagine, serine, and threonine, or a constrained amino acid selected from the group consisting of proline and histidine; residue at position 3 is an aliphatic or non-polar amino acid selected from the group consisting of alanine, leucine, isoleucine, valine, glycine, and methionine, or an aromatic amino acid selected from phenylalanine, tyrosine, or tryptophan; residue at position 6 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 7 is a polar amino acid selected from the group consisting of asparagine, glutamine, serine, and threonine, or a constrained amino acid selected from the group consisting of proline and histidine; residue at position 8 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 10 is an aliphatic or non-polar amino acid selected from the group consisting of alanine, leucine, isoleucine, valine, glycine, and methionine, or an aromatic amino acid selected from phenylalanine, tyrosine, or tryptophan; residue at position 11 is a constrained amino acid selected from the group consisting of proline and histidine; residue at position 14 is an aromatic amino acid selected from phenylalanine, tyrosine, or tryptophan; residue at position 16 is an aliphatic or non-polar amino acid selected from the group consisting of alanine, leucine, isoleucine, valine, glycine, and methionine; residue at position 22 is an aliphatic or non-polar amino acid selected from the group consisting of alanine, leucine, isoleucine, valine, glycine, and methionine, or a basic amino acid selected from the group consisting of lysine and arginine; residue at position 23 is a basic amino selected from the group consisting of lysine and arginine, or a non-polar amino acid selected from the group consisting of alanine, leucine, isoleucine, valine, glycine, and methionine, or a polar amino acid selected from the group consisting of asparagine, glutamine, serine, and threonine; residue at position 26 is a polar amino acid selected from the group consisting of asparagine, glutamine, serine, and threonine; residue at position 27 is a non-polar amino acid selected from the group consisting of alanine, leucine, isoleucine, valine, glycine, and methionine, or an acidic amino acid selected from aspartic acid and glutamic acid; residue at position 31 is a cysteine, or an acidic amino acid selected from aspartic acid and glutamic acid, or a polar amino acid selected from the group consisting of asparagine, glutamine, serine, and threonine; residue at position 33 is an aliphatic or non-polar amino acid selected from the group consisting of alanine, leucine, isoleucine, valine, glycine, and methionine; residue at position 36 is an aliphatic or non-polar amino acid selected from the group consisting of alanine, leucine, isoleucine, valine, glycine, and methionine, or a constrained amino acid selected from the group consisting of proline and histidine; residue at position 37 is a constrained amino acid selected from the group consisting of proline and histidine; residue at position 40 is an aliphatic or non-polar amino acid selected from the group consisting of alanine, leucine, isoleucine, valine, glycine, and methionine, or a cysteine; residue at position 44 is an aliphatic or non-polar amino acid selected from the group consisting of alanine, leucine, isoleucine, valine, glycine, and methionine, or a polar amino acid selected from the group consisting of asparagine, glutamine, serine, and threonine, or a constrained amino acid selected from the group consisting of proline and histidine; residue at position 46 is an aliphatic or non-polar amino acid selected from the group consisting of alanine, leucine, isoleucine, valine, glycine, and methionine, or a polar amino acid selected from the group consisting of asparagine, glutamine, and serine, or an acidic amino acid selected from aspartic acid and glutamic acid; residue at position 56 is cysteine or a constrained amino acid selected from the group consisting of proline and histidine; residue at position 57 is an aliphatic or non-polar amino acid selected from the group consisting of alanine, leucine, isoleucine, valine, glycine, and methionine; residue at position 58 is an aliphatic or non-polar amino acid selected from the group consisting of alanine, leucine, isoleucine, valine, glycine, and methionine; residue at position 87 is a polar amino acid selected from the group consisting of asparagine, glutamine, serine, and threonine; residue at position 90 is a basic amino acid selected from the group consisting of lysine and arginine; residue at position 95 is a polar amino acid selected from the group consisting of asparagine, glutamine, serine, and threonine, or a basic amino acid selected from the group consisting of lysine and arginine; residue at position 98 is an aliphatic or non-polar amino acid selected from the group consisting of alanine, leucine, valine, glycine, and methionine, or a basic amino acid selected from the group consisting of lysine and arginine; residue at position 104 is a polar amino acid selected from the group consisting of asparagine, glutamine, serine, and threonine; residue at position 105 is a polar amino acid selected from the group consisting of asparagine, glutamine, serine, and threonine, or an aromatic amino acid selected from phenylalanine, tyrosine, or tryptophan; residue at position 122 is an aliphatic or non-polar amino acid selected from the group consisting of alanine, leucine, isoleucine, glycine, and methionine; residue at position 127 is an acidic amino acid selected from aspartic acid and glutamic acid, or a basic amino acid selected from the group consisting of lysine and arginine, or an aromatic amino acid selected from phenylalanine, tyrosine, or tryptophan; residue at position 131 is a polar amino acid selected from the group consisting of asparagine, glutamine, serine, and threonine; residue at position 136 is a polar amino acid selected from the group consisting of asparagine, glutamine, serine, and threonine; residue at position 137 is an aliphatic or non-polar amino acid selected from the group consisting of alanine, leucine, isoleucine, valine, glycine, and methionine; residue at position 138 is a polar amino acid selected from the group consisting of asparagine, glutamine, serine, and threonine; residue at position 139 is an aliphatic or non-polar amino acid selected from the group consisting of alanine, leucine, isoleucine, valine, glycine, and methionine; residue at position 142 is a polar amino acid selected from the group consisting of asparagine, glutamine, serine, and threonine; residue at position 147 is a polar amino acid selected from the group consisting of asparagine, glutamine, serine, and threonine, or a constrained amino acid selected from the group consisting of proline and histidine; residue at position 149 is a polar amino acid selected from the group consisting of asparagine, glutamine, serine, and threonine; residue at position 156 is a polar amino acid selected from the group consisting of asparagine, glutamine, serine, and threonine; residue at position 161 is a polar amino acid selected from the group consisting of asparagine, glutamine, or serine; residue at position 165 is a polar amino acid selected from the group consisting of asparagine, glutamine, serine, and threonine, or a basic amino acid selected from the group consisting of lysine and arginine; residue at position 191 is a constrained amino acid selected from the group consisting of proline and histidine; residue at position 194 is an aliphatic or non-polar amino acid selected from the group consisting of alanine, leucine, isoleucine, valine, glycine, and methionine, or an acidic amino acid selected from aspartic acid and glutamic acid; residue at position 195 is a non-polar amino acid selected from the group consisting of alanine, leucine, isoleucine, valine, glycine, and methionine; residue at position 203 is an aliphatic or non-polar amino acid selected from the group consisting of alanine, leucine, isoleucine, valine, glycine, and methionine; residue at position 204 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 208 is an aliphatic or non-polar amino acid selected from the group consisting of alanine, leucine, isoleucine, valine, glycine, and methionine; residue at position 212 is a basic amino acid selected from the group consisting of arginine and lysine, or a non-polar amino acid selected from the group consisting of alanine, leucine, isoleucine, valine, glycine, and methionine; residue at position 213 is an aliphatic or non-polar amino acid selected from the group consisting of alanine, leucine, isoleucine, valine, glycine, and methionine; and residue at position 214 is a cysteine, or an acidic amino acid selected from aspartic acid and glutamic acid, or an aliphatic or non-polar amino acid selected from the group consisting of alanine, leucine, isoleucine, valine, glycine, and methionine, or a basic amino acid selected from the group consisting of lysine and arginine, or an aromatic amino acid selected from phenylalanine, tyrosine, or tryptophan, or a constrained amino acid selected from the group consisting of proline and histidine.

In certain embodiments, the present disclosure provides a recombinant carbonic anhydrase polypeptide having an improved enzyme property relative to the reference sequence of SEQ ID NO:2, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO:2, and at least one of the following amino acid substitutions at the position corresponding to the indicated position of SEQ ID NO:2: residue at position 2 is alanine, histidine, asparagine, or proline; residue at position 3 is alanine, leucine, or tryptophan; residue at position 6 is methionine, or glutamine; residue at position 7 is proline, or serine; residue at position 8 is alanine, or glutamine; residue at position 10 is valine, or tryptophan; residue at position 11 is proline; residue at position 14 is phenylalanine; residue at position 16 is valine; residue at position 22 is isoleucine, or lysine; residue at position 23 is glycine, lysine, or serine; residue at position 26 is serine; residue at position 27 is glutamic acid, or leucine; residue at position 31 is cysteine, aspartic acid, or glutamine; residue at position 33 is glycine; residue at position 36 is alanine, or histidine; residue at position 37 is histidine; residue at position 40 is cysteine, or valine; residue at position 44 is alanine, proline, or glutamine; residue at position 46 is aspartic acid, leucine, serine, or valine; residue at position 56 is cysteine, or histidine; residue at position 57 is valine; residue at position 58 is valine; residue at position 87 is threonine; residue at position 90 is lysine; residue at position 95 is glutamine; residue at position 98 is lysine, or valine; residue at position 104 is glutamine; residue at position 105 is threonine, or tryptophan; residue at position 122 is isoleucine; residue at position 127 is glutamic acid, arginine, or tryptophan; residue at position 131 is asparagine; residue at position 136 is glutamine; residue at position 137 is glycine; residue at position 138 is serine; residue at position 139 is methionine, or valine; residue at position 142 is glutamine; residue at position 147 is alanine, or histidine; residue at position 149 is serine; residue at position 156 is threonine; residue at position 161 is asparagine; residue at position 165 is asparagine, or lysine; residue at position 191 is proline; residue at position 194 is alanine, glutamic acid, or glycine; residue at position 195 is methionine; residue at position 203 is isoleucine; residue at position 204 is glycine, glutamine, or threonine; residue at position 208 is valine; residue at position 212 is arginine, glycine, or lysine; residue at position 213 is leucine; and residue at position 214 is cysteine, aspartic acid, glutamic acid, histidine, lysine, methionine, or tryptophan.

In certain embodiments, the present disclosure provides a recombinant carbonic anhydrase polypeptide having an improved enzyme property relative to a reference polypeptide of SEQ ID NO:2, an amino acid sequence having at least 80% identity to SEQ ID NO:2, wherein the amino acid sequence comprises one or more of the following amino acid substitutions at the position corresponding to the indicated position of SEQ ID NO: 2: Q2A; Q2H; Q2N; Q2P; E3A; E3L; E3W; V6M; V6Q; D7P; D7S; E8A; E8Q; S10V; S10W; N11P; E14F; P16V; P22I; P22K; E23G; E23K; E23S; A26S; P27E; P27L; P31C; P31D; P31Q; A33G; D36A; D36H; P37H; S40C; S40V; E44A; E44P; E44Q; T46D; T46L; T46S; T46V; M56C; M56H; A57V; S58V; P66G; I87T; E90K; E95K; E95Q; I98K; I98V; K104Q; E105T; E105W; V122I; A127E; A127R; A127W; D131N; M136Q; Q137G; A138S; F139M; F139V; K142Q; N147A; N147H; C149S; A156T; T161N; G165K; G165N; A191P; H194A; H194E; H194G; T195M; N203I; V204Q; V204T; E208V; E212G; E212K; E212R; T213L; S214C; S214D; S214E; S214H; S214K; S214M; S214W.

In certain embodiments, the disclosure provides a recombinant carbonic anhydrase polypeptide having an improved enzyme property relative to a reference polypeptide of SEQ ID NO:2 which comprises an amino acid sequence selected from the group consisting of SEQ ID NO: 4, 6, 10, 12, 14, 16, 20, 22, 24, 28, 36, 38, 44, 50, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In some embodiments, a carbonic anhydrase polypeptide of the present disclosure comprises a sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to a portion of the reference sequence of SEQ ID NO:2, the portion comprising a contiguous sequence of 25, 50, 75, 100, or more than 100 contiguous amino acids of SEQ ID NO:2.

In certain embodiments, the recombinant carbonic anhydrase polypeptide of the present disclosure comprises a sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the reference sequence of SEQ ID NO:2, and further comprises additional amino acids at the amino terminus and/or the carboxyl terminus. In some embodiments, the additional amino acids comprise a carboxy terminal fusion of any one of the polypeptides of SEQ ID NOs: 101-118, 316-338, the tri-peptide KAK, the dipeptide KA, or the single amino acid K.

Accordingly, in certain embodiments, a recombinant carbonic anhydrase polypeptide of the present disclosure (1) comprises a sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the reference sequence of SEQ ID NO:2, (2) comprises additional amino acids at the amino terminus and/or the carboxyl terminus, in some embodiments, from about 5 to about 40, from about 10 to about 30, or about 20 additional amino acids at the carboxyl terminus, or in some embodiments an additional 21 amino acid carboxy terminal fusion, and (3) has, at the position corresponding to the indicated position of SEQ ID NO:2, at least one of the following above-listed amino acid substitutions. In some embodiments, the additional amino acids comprise a carboxy terminal fusion of any one of the polypeptides of SEQ ID NOs: 101-118, 316-338, the tri-peptide KAK, the dipeptide KA, or the single amino acid K. In certain embodiments, the carboxy terminal fusion comprises a polypeptide of any one of SEQ ID NOs: 101, 102, 103, 104, 105, 106, 107, 108, 109, 110, 111, 112, 114, 115, 116, 118, 316, 317, 318, 319, 320, 321, 322, 323, 324, 325, 326, 327, 328, 329, 330, 331, 332, 333, 334, 335, 336, 337, or 338.

In some embodiments, the carbonic anhydrase polypeptides of the disclosure are improved as compared to SEQ ID NO: 2 with respect to their rate of enzymatic activity, i.e., their rate at which they catalyze either the forward (hydration) or reverse (dehydration) reaction, as depicted in Scheme 1. In some embodiments, the recombinant carbonic anhydrase polypeptides are equivalent to or increased at least 1.2-times, 1.5-times, 2-times, 3-times, 4-times, 5-times, 6-times, or more as compared to a reference polypeptide (e.g., wild-type of SEQ ID NO: 2, or a recombinant carbonic anhydrase polypeptide of SEQ ID NO: 24, 100, or 120) with respect to their enzymatic activity, i.e., their rate or ability of converting the substrate to the product. The present disclosure provides exemplary recombinant carbonic anhydrase polypeptides capable of converting the substrate to the product at a rate that is equivalent to or improved over a reference polypeptide, wherein the polypeptides comprise an amino acid sequence having at least 80% identity to SEQ ID NO: 2 and one or more of the above-listed amino acid substitutions. Such exemplary recombinant carbonic anhydrase polypeptide include but are not limited to, polypeptides that comprise the amino acid sequences corresponding to any one of SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In some embodiments, an improved carbonic anhydrase comprises an amino acid sequence that is at least about 85%, 86%, 87%, 88%, 89%, 90%, 91%, 92%, 93%, 94%, 95%, 96%, 97%, 98%, or 99% identical to the amino acid sequence corresponding to SEQ ID NO: 2, wherein the improved carbonic anhydrase polypeptide amino acid sequence includes any one or more of the amino acid substitutions, or combinations of substitutions, presented in Table 2. In some embodiments, these carbonic anhydrase polypeptides can have mutations at other amino acid residues, and/or insertions, deletions at other positions, and/or additional amino or carboxy terminal extensions.

In some embodiments, an improved carbonic anhydrase comprises an amino acid sequence that is at least about 85%, 86%, 87%, 88%, 89%, 90%, 91%, 92%, 93%, 94%, 95%, 96%, 97%, 98%, or 99% identical to the amino acid sequence corresponding to SEQ ID NO: 2, wherein the improved carbonic anhydrase polypeptide amino acid sequence includes any one set of the specified amino acid substitution combinations presented in Table 2 and a carboxy terminal fusion of any one of the polypeptides of SEQ ID NOs: 101-118, 316-338, the tri-peptide KAK, the dipeptide KA, or the single amino acid K. In some embodiments, these carbonic anhydrase polypeptides can have mutations at other amino acid residues.

In certain embodiments, as compared to the wild-type enzyme of SEQ ID NO: 2, the recombinant carbonic anhydrase polypeptides of the present disclosure exhibit the improved property of increased rate of hydrating carbon dioxide to bicarbonate as in Scheme 1, wherein the increased rate is determined under specified conditions.

In some embodiments, this improvement of increased rate (or activity) can be determined in the presence of basic solvents, e.g., the recombinant carbonic anhydrase polypeptides of the present disclosure retain substantially more enzymatic activity when assayed in the presence of CO₃⁻² at a concentration within a range of from about 0.1 M CO₃⁻² to about 5 M CO₃⁻², from about 0.2 M CO₃⁻² to about 4 M CO₃⁻², or from about 0.3 M CO₃⁻² to about 3 M CO₃⁻².

In some embodiments, the rate can be determined in the presence of an aqueous solution (e.g., a buffered solution), a solvent solution (e.g., an organic solvent), or co-solvent solution (e.g., an aqueous-organic co-solvent system). In some embodiments, the rate can be determined in the presence of a co-solvent selected from the group consisting of: monoethanolamine (MEA), methyldiethanolamine (MDEA), 2-aminomethylpropanolamine (AMP), 2-(2-aminoethylamino)ethanol (AEE), triethanolamine, 2-amino-2-hydroxymethyl-1,3-propanediol (Tris), dimethyl ether of polyethylene glycol (PEG DME), piperazine, ammonia, and mixtures thereof. In some embodiments, the rate can be determined in the presence of from about 0.5 M AMP to about 3.0 M AMP, from about 1.0 M AMP to about 2.0 M AMP, or from about 1.25 M AMP to about 1.75 M AMP.

In some embodiments, the rate can be determined in the presence of a solution at a basic pH such as a pH of from about pH 8 to about pH 12, from about pH 9 to about pH 11.5, or from about pH 9.5 to pH 11.

In certain embodiments, as compared to the wild-type enzyme of SEQ ID NO: 2, the recombinant carbonic anhydrase polypeptides of the present disclosure exhibit increased thermotolerance (e.g., thermostability). That is, the recombinant carbonic anhydrase polypeptides of the present disclosure retain substantially more enzymatic activity after exposure to a temperature within the range of from about 50° C. to about 100° C., or within the range of from about 60° C. to about 90° C., or within a range of from 70° C. to about 80° C.

In another aspect, the present disclosure provides polynucleotides encoding the engineered carbonic anhydrases described herein or polynucleotides that hybridize to such polynucleotides under highly stringent conditions. The polynucleotide can include promoters and other regulatory elements useful for expression of the encoded engineered carbonic anhydrases, and can utilize codons optimized for specific desired expression systems. In some embodiments, the polynucleotides encode a carbonic anhydrase polypeptide having at least the following amino acid sequence as compared to the amino acid sequence of SEQ ID NO: 2, and further comprising at least one acid substitution selected from the group of amino acid substitutions and additions provided in Table 2. In some embodiments, the polynucleotides encoding an engineered carbonic anhydrase comprise a nucleotide sequence having one or more of the following nucleotide substitutions relative to SEQ ID NO: 119: a537g; t160a; a300g; g48t; c165t; a333t; a217t; t453g; t618g; c612t. Exemplary polynucleotides include, but are not limited to, a polynucleotide sequence of any of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 304, 305, 306, 307, 308, 309, 310, 311, and 312.

In another aspect, the present disclosure provides host cells comprising the polynucleotides and/or expression vectors described herein. The host cells may be M. thermophila or they may be a different organism, such as E. coli, Saccharomyces cerevisiae, Bacillus spp. (e.g., B. amyloliquefaciens, B. licheniformis, B. megaterium, B. stearothermophilus, and B. subtilis), or filamentous fungal organisms such as Aspergillus spp. including but not limited to A. niger, A. nidulans, A. awamori, A. oryzae, A. sojae and A. kawachi; Trichoderma reesei; Chrysosporium lucknowense; Myceliophthora thermophilia; Fusarium venenatum; Neurospora crassa; Humicola insolens; Humicola grisea; Penicillum verruculosum; Thielavia terrestris; and teleomorphs, or anamorphs and synonyms or taxonomic equivalents thereof. The host cells can be used for the expression and isolation of the engineered carbonic anhydrase enzymes described herein, or, alternatively, they can be used directly for carrying out the reactions of Scheme 1.

In some embodiments, the disclosure provides a method of producing a recombinant carbonic anhydrase polypeptide of the present disclosure, wherein said method comprises the steps of: (a) transforming a host cell with an expression vector polynucleotide encoding the recombinant carbonic anhydrase polypeptide; (b) culturing said transformed host cell under conditions whereby said recombinant carbonic anhydrase polypeptide is produced by said host cell; and (c) recovering said recombinant carbonic anhydrase polypeptide from said host cells. In some embodiments, the method of producing the recombinant carbonic anhydrase may be carried out wherein said expression vector comprises a secretion signal, and said cell is cultured under conditions whereby the recombinant carbonic anhydrase polypeptide is secreted from the cell. In some embodiments of the method, the expression vector comprises a polynucleotide encoding a secretion signal. In some embodiments, the secretion signal encodes a signal peptide is selected from SEQ ID NO: 313, 314, and 315.

In some embodiments, the recombinant carbonic anhydrase polypeptides of the present disclosure are used in methods for the absorption and/or desorption of carbon dioxide produced, for example, by the combustion of fossil fuels. In one aspect of this embodiment, a recombinant carbonic anhydrase polypeptide of the present disclosure is used to catalyze the hydration of carbon dioxide absorbed in a solution so as to provide a solution comprising bicarbonate and/or carbonate ions (depending on the pH of that solution). The bicarbonate and/or carbonate containing solution can be recovered (e.g., isolated) and contacted with a recombinant carbonic anhydrase polypeptide of the present disclosure to release the carbon dioxide. In some aspects of this embodiment, the recombinant carbonic anhydrase polypeptides of the present disclosure are immobilized on a solid surface and one or both of the hydration and dehydration reactions is carried out in a bioreactor comprising the immobilized polypeptides. In other aspects of this embodiment, the hydration reaction is performed at a relatively alkaline pH while the dehydration is carried out at a relatively acidic pH.

In some embodiments, the present disclosure provides a method for removing carbon dioxide from a gas stream comprising the step of contacting the gas stream with a solution comprising a recombinant carbonic anhydrase polypeptide having an improved property of the disclosure, whereby carbon dioxide from the gas stream is dissolved in the solution and converted to hydrated carbon dioxide. In certain embodiments, the method is carried out wherein the solution is aqueous, or an aqueous co-solvent system. In some embodiments of the method, the solution used is an aqueous co-solvent system comprising a co-solvent selected from: monoethanolamine (MEA), methyldiethanolamine (MDEA), 2-aminomethylpropanolamine (AMP), 2-(2-aminoethylamino)ethanol (AEE), triethanolamine, 2-amino-2-hydroxymethyl-1,3-propanediol (Tris), dimethyl ether of polyethylene glycol (PEG DME), piperazine, ammonia, and mixtures thereof.

In any of the above embodiments, the methods can be carried out wherein the recombinant carbonic anhydrase polypeptide is immobilized on a surface, for example a surface on a particle in the solution. In another embodiment of the above methods, the method further comprises the step of isolating the solution comprising the hydrated carbon dioxide and contacting the isolated solution with hydrogen ions and a recombinant carbonic anhydrase polypeptide, thereby converting the hydrated carbon dioxide to carbon dioxide gas and water.

Whether carrying out the method with whole cells, cell extracts or purified carbonic anhydrase enzymes, a single carbonic anhydrase enzyme may be used or, alternatively, mixtures of two or more recombinant carbonic anhydrase enzymes of the present disclosure may be used.

### BRIEF DESCRIPTION OF THE FIGURES

FIG. 1 illustrates the improvement in activity of the carbonic anhydrase polypeptide of SEQ ID NO:24 in the presence of increasing concentrations of carbonate buffer, as compared to that of the parent wild type enzyme of SEQ ID NO:2.

FIG. 2 illustrates the improvement in activity of the carbonic anhydrase polypeptide of SEQ ID NO:24 in the presence of increasing concentrations of carbonate, as compared to that of the parent wild type enzyme of SEQ ID NO:2, after exposure to elevated temperature.

FIG. 3 illustrates the improvement in activity of the carbonic anhydrase polypeptides of and SEQ ID NO: 4 (“H108”) and SEQ ID NO: 24 (“H101”), as compared to that of the parent wild type enzyme of SEQ ID NO: 2 (“WT”), as well as the relative activity of four other isolates SEQ ID NO: 36 (“H104”), SEQ ID NO: 50 (“H105”), and SEQ ID NO: 56 (“H106”), either with or without prior exposure to elevated temperature.

FIG. 4 depicts results of thermostability assays of members of a C-terminal extension truncation library based on SEQ ID NO: 24. Each polypeptide was incubated for 30 minutes at 75° C. in 150 mM K2CO₃, pH 10.9 and then assayed with 400 μM phenolphthalein, 150 mM K2CO₃, pH 10.9. The recombinant carbonic anhydrase polypeptide of SEQ ID NO: 24 (denoted “G05” with star in figure) includes a 21 amino acid C-terminal extension (begins after position 214). G05-1 represents the recombinant carbonic anhydrase polypeptide of SEQ ID NO: 24 with its 21 amino acid C-terminal extension truncated by 1 amino acid. Similarly, G05-2 through G05-20 each represents a further 1 amino acid truncation of the 21 amino acid extension of SEQ ID NO: 24. Accordingly, G05-21 is a recombinant carbonic anhydrase polypeptide of SEQ ID NO: 24 without any C-terminal extension beyond position 214. All values were the average of four assays (N=4) except for G05 where N=6. Variants above the top horizontal bar exhibited increased thermostability relative to SEQ ID NO: 24 (G05). Variants below the lower horizontal bar exhibited decreased thermostability compared to SEQ ID NO: 24. However, all exhibited improved thermostability over the polypeptide without any C-terminal extension (G05-21).

### DETAILED DESCRIPTION

The present disclosure is directed to recombinant carbonic anhydrases having improved properties, particularly as compared to those of their parent, the carbonic anhydrase of SEQ ID NO: 2. The present disclosure is also directed to the use of such carbonic anhydrases in methods for the capture and sequestration of carbon dioxide generated by combustion of fossil fuel. The present disclosure is further directed to the use of such carbonic anhydrases in bioreactors useful for not only for sequestration (hydration) of carbon dioxide generated by fossil fuel burning power plants but also for the subsequent recovery (dehydration) of that previously sequestered carbon dioxide.

### 7.1. DEFINITIONS

As used herein, the following terms are intended to have the following meanings:

“Carbonic anhydrase” and “CA” are used interchangeably herein to refer to a polypeptide having an enzymatic capability of carrying out the reactions depicted in Scheme 1. Carbonic anhydrase as used herein include naturally occurring (wild type) carbonic anhydrases as well as non-naturally occurring engineered polypeptides generated by human manipulation.

“Protein”, “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Polynucleotide” or “nucleic Acid’ refers to two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. Non-limiting example of such non-standard linkages include phosphoramidates (Beaucage et al., 1993, Tetrahedron 49:1925; Letsinger, 1970, Nucl. Acids. Res. 14:3487; Sawai et al, 1984, Chem Lett N5:805-808; Letsinger et al., 1988, J. Am. Chem. Soc. 110:4470; Pauwels et al., 1986, Chemica Scripta 26:141), phosphorothioates (Mag et al., 1991, Nucl. Acids. Res. 19:1437; U.S. Pat. No. 5,644,048), phosphorothioates (Briu et al., 1989, J. Am. Chem. Soc. 111:2321), O-methylphosphodiesters (Eckstein, 1991, Oligonucleotides and Analogues: A Practical Approach, Oxford University Press), amides (Egholm, 1992, J. Am. Chem. Soc. 114:1895; Meier et al., 1992, Chem. Rut. Ed. Engl. 31:1008; Nielson, 1993, Nature 365:366; Carlsson et al., 1996, Nature 380:207 WO 94/25477; WO 92/20702; U.S. Pat. Nos. 6,107,470; 5,786,461; 5 773 571; 5 719 262; and 5 539 082), positively-charged linkages (Denocy et al., 1995, Proc. Natl. Acad. Sci. USA 92:6097 and non-ionic linkages (U.S. Pat. Nos. 5,386,023; 5,637,684; 5,602,240; 5,216,141; and 4,469 863); Kiedrowski et al., 1991, Angew. Chem. Intl. Ed. English 30:423; Letsinger et al. 1988, J. Am. Chem. Soc. 110:4470; and Letsinger et al. 1994, Nucleosides & Nucleotides 13:1597).

“Coding sequence” refers to that portion of a nucleic acid (e.g., a gene) that encodes an amino acid sequence of a protein.

“Naturally occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Recombinant” or “engineered” or “non-naturally occurring” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. Mol. Biol. 215: 403-410 and Altschul et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison Wis.), using default parameters provided.

“Reference sequence” refers to a defined sequence to which another sequence is compared. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity.

The term “reference sequence” is not intended to be limited to wild-type sequences, and can include engineered or altered sequences. For example, in some embodiments, a “reference sequence” can be a previously engineered or altered amino acid sequence.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Substantial identity” refers to a polynucleotide or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered carbonic anhydrase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Improved enzyme property” refers to any enzyme property made better or more desirable for a particular purpose as compared to that property found in a reference enzyme. For the engineered carbonic anhydrase polypeptides described herein, the comparison is generally made to the wild-type carbonic anhydrase enzyme of SEQ ID NO: 2, although in some embodiments, the reference carbonic anhydrase could be another naturally occurring or an engineered carbonic anhydrase (e.g., the recombinant polypeptides of SEQ ID NO: 4, 24 or 120). Enzyme properties for which improvement is desirable include, but are not limited to, enzymatic activity (which can be expressed in terms of percent conversion of the substrate in a period of time), thermal stability, pH activity profile, refractoriness to inhibitors, e.g., product inhibition, substrate inhibition or inhibition by a component of the feedstock (e.g. exhaust, flue gas etc.) comprising carbon dioxide, bicarbonate, or carbonate, as well as increased expression of active enzyme, increased stability and/or activity in the presence of additional reagents useful for absorption or sequestration of carbon dioxide, including, for example, calcium ions, monoethanolamine, methyldiethanolamine, and 2-aminomethylpropanolamine.

“Increased enzymatic activity” or “increased activity” refers to an improved property of the engineered enzyme (e.g., carbonic anhydrase), which can be represented by an increase in specific activity (e.g., product produced/time/weight protein) or an increase in percent conversion of the substrate to the product (e.g., percent conversion of carbon dioxide to bicarbonate and/or carbonate in a specified time period using a specified amount of carbonic anhydrase) as compared to a reference enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1-times the enzymatic activity of the corresponding wild-type carbonic anhydrase enzyme, to as much as 1.2-times, 1.5-times, 2-times, 3-times, 4-times, 5-times, 6-times, 7-times, or more than 8-times the enzymatic activity than the naturally occurring parent carbonic anhydrase. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M⁻¹ s⁻¹). Hence, any improvements in the enzyme activity of the carbonic anhydrase will have an upper limit related to the diffusion rate of the substrates acted on by the carbonic anhydrase enzyme. Carbonic anhydrase activity can be measured by any one of standard assays used for measuring carbonic anhydrase, e.g., as provided in the Examples. Comparisons of enzyme activities are made, e.g., using a defined preparation of enzyme, a defined assay under a set of conditions, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Conversion” refers to the enzymatic conversion of the substrate to the corresponding product. “Percent conversion” refers to the percent of the substrate that is reduced to the product within a period of time under specified conditions. Thus, the “enzymatic activity” or “activity” of a carbonic anhydrase polypeptide can be expressed as “percent conversion” of the substrate to the product.

“Thermostable” refers to a carbonic anhydrase polypeptide that maintains similar activity (more than 60% to 80% for example) after exposure to elevated temperatures (e.g. 55-100° C.) for a period of time (e.g. 0.5-24 hrs) compared to the untreated enzyme.

“Solvent stable” refers to a carbonic anhydrase polypeptide that maintains similar activity (more than e.g., 60% to 80%) after exposure to varying concentrations (e.g., 5-99%) of solvent or other reaction component (e.g., monoethanolamine, methyldiethanolamine, and 2-aminomethylpropanolamine) for a period of time (e.g., 0.5-24 hrs) compared to the untreated enzyme.

“pH stable” refers to a carbonic anhydrase polypeptide that maintains similar activity (more than e.g., 60% to 80%) after exposure to high or low pH (e.g., 8 to 12, or 4.5 to 6) for a period of time (e.g., 0.5-24 hrs) compared to the untreated enzyme.

“Thermo- and solvent stable” refers to a carbonic anhydrase polypeptide that is both thermostable and solvent stable.

“Derived from” as used herein in the context of engineered carbonic anhydrase enzymes, identifies the originating carbonic anhydrase enzyme, and/or the gene encoding such carbonic anhydrase enzyme, upon which the engineering was based.

“Amino acid” or “residue” as used in context of the polypeptides disclosed herein refers to the specific monomer at a sequence position (e.g., D7 indicates that the “amino acid” or “residue” at position 7 of SEQ ID NO: 2 is an aspartic acid (D).)

“Hydrophilic Amino Acid or Residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L Ser (S), L His (H), L Glu (E), L Asn (N), L Gln (O), L Asp (D), L Lys (K) and L Arg (R).

“Acidic Amino Acid or Residue” refers to a hydrophilic amino acid or residue having a side chain exhibiting a pKa value of less than about 6 when the amino acid is included in a peptide or polypeptide. Acidic amino acids typically have negatively charged side chains at physiological pH due to loss of a hydrogen ion. Genetically encoded acidic amino acids include L Glu (E) and L Asp (D).

“Basic Amino Acid or Residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L Arg (R) and L Lys (K).

“Polar Amino Acid or Residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L Asn (N), L Gln (O), L Ser (S) and L Thr (T).

“Hydrophobic Amino Acid or Residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L Pro (P), L Ile (I), L Phe (F), L Val (V), L Leu (L), L Trp (W), L Met (M), L Ala (A) and L Tyr (Y).

“Aromatic Amino Acid or Residue” refers to a hydrophilic or hydrophobic amino acid or residue having a side chain that includes at least one aromatic or heteroaromatic ring. Genetically encoded aromatic amino acids include L Phe (F), L Tyr (Y) and L Trp (W). Although owing to the pKa of its heteroaromatic nitrogen atom L His (H) it is sometimes classified as a basic residue, or as an aromatic residue as its side chain includes a heteroaromatic ring, herein histidine is classified as a hydrophilic residue or as a “constrained residue” (see below).

“Constrained amino acid or residue” refers to an amino acid or residue that has a constrained geometry. Herein, constrained residues include L pro (P) and L his (H). Histidine has a constrained geometry because it has a relatively small imidazole ring. Proline has a constrained geometry because it also has a five membered ring.

“Non-polar Amino Acid or Residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L Gly (G), L Leu (L), L Val (V), L Ile (I), L Met (M) and L Ala (A).

“Aliphatic Amino Acid or Residue” refers to a hydrophobic amino acid or residue having an aliphatic hydrocarbon side chain. Genetically encoded aliphatic amino acids include L Ala (A), L Val (V), L Leu (L) and L Ile (I).

“Cysteine” or the amino acid L Cys (C) is unusual in that it can form disulfide bridges with other L Cys (C) amino acids or other sulfanyl- or sulfhydryl-containing amino acids. The “cysteine-like residues” include cysteine and other amino acids that contain sulfhydryl moieties that are available for formation of disulfide bridges. The ability of L Cys (C) (and other amino acids with SH containing side chains) to exist in a peptide in either the reduced free SH or oxidized disulfide-bridged form affects whether L Cys (C) contributes net hydrophobic or hydrophilic character to a peptide. While L Cys (C) exhibits a hydrophobicity of 0.29 according to the normalized consensus scale of Eisenberg (Eisenberg et al., 1984, supra), it is to be understood that for purposes of the present disclosure L Cys (C) is categorized into its own unique group.

“Small Amino Acid or Residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L Ala (A), L Val (V), L Cys (C), L Asn (N), L Ser (S), L Thr (T) and L Asp (D).

“Hydroxyl-containing Amino Acid or Residue” refers to an amino acid containing a hydroxyl (—OH) moiety. Genetically-encoded hydroxyl-containing amino acids include L Ser (S) L Thr (T) and L-Tyr (Y).

“Conservative” amino acid substitutions or mutations refer to the interchangeability of residues having similar side chains, and thus typically involves substitution of the amino acid in the polypeptide with amino acids within the same or similar defined class of amino acids. However, as used herein, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 1 below shows exemplary conservative substitutions.

TABLE 1
Conservative Substitutions

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P, H | Other constrained (P, H) |
| N, Q, S, T | Other polar (N, Q, S, T) |
| Y, W, F | Other aromatic (Y, W, F) |
| C | None |

“Non-conservative substitution” refers to substitution or mutation of an amino acid in the polypeptide with an amino acid with significantly differing side chain properties. Non-conservative substitutions may use amino acids between, rather than within, the defined groups listed above. In one embodiment, a non-conservative mutation affects (a) the structure of the peptide backbone in the area of the substitution (e.g., proline for glycine) (b) the charge or hydrophobicity, or (c) the bulk of the side chain.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered carbonic anhydrase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. In some embodiments, the improved engineered carbonic anhydrase enzymes comprise insertions of one or more amino acids to the naturally occurring carbonic anhydrase polypeptide as well as insertions of one or more amino acids to other improved carbonic anhydrase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Different from” or “differs from” with respect to a designated reference sequence refers to difference of a given amino acid or polynucleotide sequence when aligned to the reference sequence. Generally, the differences can be determined when the two sequences are optimally aligned. Differences include insertions, deletions, or substitutions of amino acid residues in comparison to the reference sequence.

“Fragment” as used herein refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long, at least 75 amino acids long, at least 100 amino acids long, or longer, and up to 70%, 80%, 90%, 95%, 98%, and 99% of the full-length carbonic anhydrase polypeptide.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The improved carbonic anhydrase enzymes may be present within a cell, present in the cellular medium, or prepared in various forms, such as lysates or isolated preparations. As such, in some embodiments, the improved carbonic anhydrase enzyme can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure carbonic anhydrase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated improved carbonic anhydrase polypeptide is a substantially pure polypeptide composition.

“Stringent hybridization” is used herein to refer to conditions under which nucleic acid hybrids are stable. As known to those of skill in the art, the stability of hybrids is reflected in the melting temperature (Tm) of the hybrids. In general, the stability of a hybrid is a function of ion strength, temperature, G/C content, and the presence of chaotropic agents. The Tm values for polynucleotides can be calculated using known methods for predicting melting temperatures (see, e.g., Baldino et al., Methods Enzymology 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res 18:6409-6412 (erratum, 1991, Nucleic Acids Res 19:698); Sambrook et al., supra); Suggs et al., 1981, In Developmental Biology Using Purified Genes (Brown et al., eds.), pp. 683-693, Academic Press; and Wetmur, 1991, Crit Rev Biochem Mol Biol 26:227-259. All publications incorporate herein by reference). In some embodiments, the polynucleotide encodes the polypeptide disclosed herein and hybridizes under defined conditions, such as moderately stringent or highly stringent conditions, to the complement of a sequence encoding an engineered carbonic anhydrase enzyme of the present disclosure.

“Hybridization stringency” relates to such washing conditions of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA; with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tm as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Other high stringency hybridization conditions, as well as moderately stringent conditions, are described in the references cited above.

“Heterologous” polynucleotide refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the carbonic anhydrases enzymes may be codon optimized for optimal production from the host organism selected for expression.

“Preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; McInerney, J. 0, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTs), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed at a position relative to the coding sequence of the DNA sequence such that the control sequence directs the expression of a polynucleotide and/or polypeptide.

“Promoter sequence” is a nucleic acid sequence that is recognized by a host cell for expression of the coding region. The control sequence may comprise an appropriate promoter sequence. The promoter sequence contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Fusion construct” refers to a nucleic acid comprising the coding sequence for a first polypeptide and the coding sequence (with or without introns) for a second polypeptide in which the coding sequences are adjacent and in the same reading frame such that, when the fusion construct is transcribed and translated in a host cell, a polypeptide is produced in which the C-terminus of the first polypeptide is joined to the N-terminus of the second polypeptide. A “fusion polypeptide” refers to the polypeptide product of the fusion construct.

### 7.2. RECOMBINANT CARBONIC ANHYDRASE ENZYMES

The recombinant (or engineered) carbonic anhydrase (“CA”) enzymes of the present disclosure are those having an improved property when compared with a naturally-occurring, wild type carbonic anhydrase enzyme obtained from Methanosarcina thermophila (SEQ ID NO: 2). Enzyme properties for which improvement is desirable include, but are not limited to, enzymatic activity, thermal stability, pH activity profile, refractoriness to inhibitors, e.g. product inhibition by bicarbonate and/or carbonate, refractoriness to inhibition by other reaction components, such as monoethanolamine (MEA), methyldiethanolamine (MDEA), and 2-aminomethylpropanolamine (AMP), and solvent stability. The improvements can relate to a single enzyme property, such as enzymatic activity, or a combination of different enzyme properties, such as enzymatic activity and thermostability.

As noted above, the amino acid residue positions of the engineered carbonic anhydrases with improved enzyme property disclosed herein are described with reference to the wild-type enzyme from Methanosarcina thermophila which is listed herein as reference polypeptide of SEQ ID NO: 2. The amino acid residue positions are determined in the recombinant carbonic anhydrases described beginning from the initiating methionine (M) residue (i.e., M represents residue position 1) of SEQ ID NO: 2, although it will be understood by the skilled artisan that this initiating methionine residue may be removed by biological processing machinery, such as in a host cell or in vitro translation system, to generate a mature protein lacking the initiating methionine residue. Consequently, the term “residue difference at position corresponding to X of SEQ ID NO: 2” as used herein may refer to position X the naturally occurring carbonic anhydrase or to the equivalent position (e.g., X-1 position) in a reference sequence that has been processed so as to lack the starting methionine.

The polypeptide sequence position at which a particular amino acid or amino acid change (e.g., a “residue difference”) is present is sometimes described herein as “Xₙ”, “Xn,” “residue n,” or “position n”, where n refers to the residue position with respect to the reference sequence. A specific substitution mutation, which is a replacement of the specific residue in a reference sequence with a different specified residue may be denoted by the conventional notation “X(number)Y”, where X is the single letter identifier of the residue in the reference sequence, “number” is the residue position in the reference sequence (e.g., the wild-type carbonic anhydrase of SEQ ID NO:2), and Y is the single letter identifier of the residue substitution in the engineered sequence. In such instances, the single letter codes are used to represent the amino acid; e.g. D7S refers to an instance in which the “wild type” amino acid residue, aspartic acid at position 7 of SEQ ID NO: 2 has been replaced with the amino acid serine.

Herein, mutations are sometimes described as a mutation of a residue “to a” type of amino acid. For example, SEQ ID NO: 2, residue 7 (aspartic acid (D)) can be mutated “to a” polar residue. But the use of the phrase “to a” does not exclude mutations from one amino acid of a class to another amino acid of the same class. For example, residue 7 can be mutated from aspartic acid “to an” asparagine.

The naturally occurring polynucleotide encoding the naturally occurring carbonic anhydrase of Methanosarcina thermophila TM-1 can be obtained from the isolated polynucleotide known to encode the carbonic anhydrase activity (e.g., Genbank Accession No. U08885).

In some embodiments, the carbonic anhydrase polypeptides herein can have a number of modifications (e.g., substitutions, insertions, and/or deletions) to the reference sequence (e.g., Methanosarcina thermophila CA polypeptide of SEQ ID NO: 2) to result in an improved carbonic anhydrase enzyme property. In such embodiments, the number of modifications to the amino acid sequence can comprise one or more amino acids, 2 or more amino acids, 3 or more amino acids, 4 or more amino acids, 5 or more amino acids, 6 or more amino acids, 8 or more amino acids, 9 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the reference enzyme sequence. In some embodiments, the number of modifications to the naturally occurring polypeptide or an engineered polypeptide that produces an improved carbonic anhydrase property may comprise from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 modifications of the reference sequence. The modifications can comprise insertions, deletions, substitutions, or combinations thereof.

In some embodiments, the modifications comprise amino acid substitutions relative to a reference sequence (e.g., the sequence of Methanosarcina thermophila carbonic anhydrase of SEQ ID NO: 2). Substitutions that can produce an improved carbonic anhydrase property may be at one or more amino acids, 2 or more amino acids, 3 or more amino acids, 4 or more amino acids, 5 or more amino acids, 6 or more amino acids, 7 or more amino acids, 8 or more amino acids, 9 or more amino acids, 10 or more amino acids, 15 or more amino acids, or 20 or more amino acids, up to 10% of the total number of amino acids, up to 15% of the total number of amino acids, up to 20% of the total number of amino acids, or up to 30% of the total number of amino acids of the reference enzyme sequence. In some embodiments, the number of substitutions to the naturally occurring polypeptide or an engineered polypeptide that produces an improved carbonic anhydrase property can comprise from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 amino acid substitutions of the reference sequence.

In some embodiments, the improved property of the carbonic anhydrase polypeptide is with respect to an increase in its ability to convert a greater percentage of the substrate to the product. In some embodiments, the improved property of the carbonic anhydrase polypeptide is with respect to an increase in its rate of conversion of the substrate to the product (e.g., hydration of carbon dioxide to bicarbonate). This improvement in enzymatic activity can be manifested by the ability to use less of the improved polypeptide as compared to the wild-type or other reference sequence(s) to reduce or convert the same amount of product. In some embodiments, the improved property of the carbonic anhydrase polypeptide is with respect to its stability or thermostability. In some embodiments, the carbonic anhydrase polypeptide has more than one improved property, such as a combination of enzyme activity and thermostability.

In some embodiments, the improved property of the recombinant carbonic anhydrase polypeptide is increased rate of hydrating carbon dioxide to bicarbonate. In some embodiments of the recombinant carbonic anhydrase polypeptides provided herein, this rate is increased at least 1.2-times, 1.5-times, 2-times, 3-times, 4-times, 5-times, 6-times, or more than that of the reference polypeptide having the amino acid sequence of SEQ ID NO: 2. In some embodiments, the rate is increased at least 1.2-times, 1.5-times, 2-times, 3-times, 4-times, 5-times, 6-times, or more than that of the reference polypeptide that is a recombinant carbonic anhydrase polypeptide (e.g., SEQ ID NO: 4, 24, or 120), that is already improved over the wild type polypeptide of SEQ ID NO: 2. In such embodiments, relative improvement over the WT is assumed. For example, where a second recombinant carbonic anhydrase polypeptide of the present disclosure has e.g., at least a 2-fold increased rate over a first recombinant carbonic anhydrase polypeptide, which in turn has a rate at least 2-fold increased over WT, it is understood that the second recombinant carbonic anhydrase polypeptide has at least 4-fold increased rate relative to the WT polypeptide.

In some embodiments where the improved property is rate of hydrating carbon dioxide to bicarbonate, the rate can be determined or measured under a range of different reaction (or assay) conditions to provide measures of various improved properties—e.g., thermal stability, solvent stability, and/or base stability. Accordingly, in some embodiments, the rate can be measured in the presence of from about 0.1 M K2CO₃ to about 5 M K2CO₃, from about 0.2 M K2CO₃ to about 4 M K2CO₃, or from about 0.3 M K2CO₃ to about 3 M K2CO₃.

In some embodiments, the rate can be determined after heating the recombinant carbonic anhydrase polypeptide and the reference polypeptide at a temperature of from about 50° C. to 100° C., from about 60° C. to 90°, or from about 70° C. to 80°, wherein said heating is for a period of time from about 5 minutes to about 180 minutes, from about 10 minutes to about 120 minutes, or from about 15 minutes to about 60 minutes.

In some embodiments, the rate can be determined under a combination of conditions, including e.g., in the presence of from about 0.1 M K2CO₃ to about 0.5 M K2CO₃ after heating the recombinant carbonic anhydrase polypeptide and the reference polypeptide at a temperature within the range of from about 50° C. to 100° C. for a period of time within the range of from about 5 minutes to about 180 minutes.

In some embodiments, the rate can be determined in the presence of an aqueous solution (e.g., a buffered solution), a solvent solution (e.g., an organic solvent), or co-solvent solution (e.g., an aqueous-organic co-solvent system). In some embodiments, the solution, or co-solvent system, comprises a solvent that thermodynamically and/or kinetically favors the absorption of CO₂. Solutions and solvent systems having improved thermodynamic and kinetic characteristics for the absorption of CO₂ are described in e.g., WO2006/089423A1, which is hereby incorporated by reference herein.

In some embodiments, the rate can be determined in the presence of a co-solvent selected from the group consisting of: monoethanolamine (MEA), methyldiethanolamine (MDEA), 2-aminomethylpropanolamine (AMP), 2-(2-aminoethylamino)ethanol (AEE), triethanolamine, 2-amino-2-hydroxymethyl-1,3-propanediol (Tris), dimethyl ether of polyethylene glycol (PEG DME), piperazine, ammonia, and mixtures thereof. In some embodiments, the rate can be determined in the presence of from about 0.5 M AMP to about 3.0 M AMP, from about 1.0 M AMP to about 2.0 M AMP, or from about 1.25 M AMP to about 1.75 M AMP.

In some embodiments, the rate can be determined in the presence of a solution at a basic pH—e.g., from about pH 8 to about pH 12. Accordingly, in some embodiments, the rate is determined at a pH of from about pH 8 to about pH 12, from about pH 9 to about pH 11.5, or from about pH 9.5 to pH 11.

In some embodiments, the recombinant carbonic anhydrase polypeptides are equivalent to or increased at least 1.2-times, 1.5-times, 2-times, 3-times, 4-times, 5-times, 6-times, or more as compared to a reference polypeptide (e.g., wild-type of SEQ ID NO: 2, or a recombinant CA of SEQ ID NO: 24, 100, or 120) with respect to their enzymatic activity, i.e., their rate or ability of converting the substrate to the product.

Exemplary polypeptides that are capable of converting the substrate to the product at a rate that is equivalent to or improved over wild-type, include but are not limited to, polypeptides that comprise the amino acid sequences corresponding to any one of SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

Exemplary polypeptides that are capable of converting the substrate to the product at a rate that is at least about 2-fold improved as compared to the wild-type, include but are not limited to, polypeptides that comprise the amino acid sequences corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 60, 62, 64, 66, 68, 70, 72, 74, 76, 78, 80, 82, 84, 86, 88, 90, 92, 94, 96, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

Exemplary polypeptides that are capable of converting the substrate to the product at a rate that is at least about 3-fold improved as compared to the wild-type, include but are not limited to, polypeptides that comprise the amino acid sequences corresponding to SEQ ID NO: 4, 6, 10, 12, 14, 16, 20, 22, 24, 28, 36, 38, 44, 50, 56, 60, 62, 64, 66, 68, 70, 72, 74, 76, 78, 80, 82, 84, 86, 88, 90, 94, 96, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

Exemplary polypeptides that are capable of converting the substrate to the product at a rate that is at least about 4-fold improved as compared to the wild-type, include but are not limited to, polypeptides that comprise the amino acid sequences corresponding to SEQ ID NO: 4, 6, 10, 16, 20, 22, 24, 60, 62, 64, 66, 68, 70, 72, 74, 76, 78, 80, 82, 84, 86, 88, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

Exemplary polypeptides that are capable of converting the substrate to the product at a rate that is at least about 5-fold improved as compared to the wild-type, include but are not limited to, polypeptides that comprise the amino acid sequences corresponding to SEQ ID NO: 4, 6, 16, 22, 24, 60, 62, 64, 66, 68, 70, 72, 74, 76, 78, 80, 84, 86, 88, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

Exemplary polypeptides that are capable of converting the substrate to the product at a rate that is at least about 6-fold improved as compared to the wild-type, include but are not limited to, polypeptides that comprise the amino acid sequences corresponding to SEQ ID NO: 4, 22, 24, 60, 62, 64, 66, 68, 70, 72, 74, 76, 78, 80, 84, 86, 88, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

Exemplary polypeptides that are capable of converting the substrate to the product at a rate that is at least about 7-fold improved as compared to the wild-type, include but are not limited to, polypeptides that comprise the amino acid sequences corresponding to SEQ ID NO: 4, 24, 60, 62, 64, 66, 68, 70, 72, 74, 76, 78, 80, 84, 86, 88, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

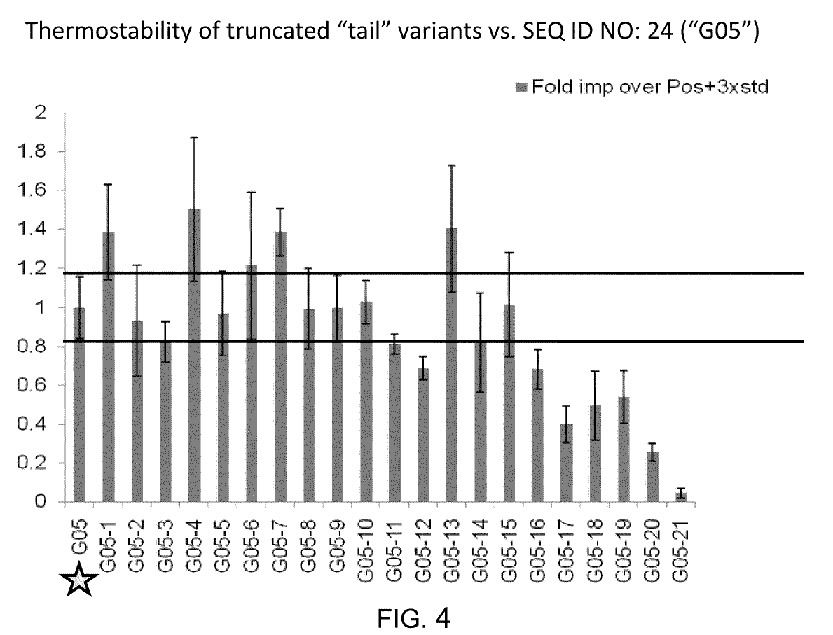

Table 2 below provides a list of the SEQ ID NOs for recombinant carbonic anhydrases disclosed herein. Examples 5-11 (including Tables 3, 5, 6 and FIG. 4) below provide improved enzyme properties, e.g., reaction rate, exhibited by the engineered polypeptides of the SEQ ID NOs disclosed herein. In Table 2 below, each row lists two SEQ ID NOs, where the odd number refers to the nucleotide sequence that encodes the amino acid sequence provided by the even number. All sequences below are derived from the wild-type Methanosarcina thermophila carbonic anhydrase sequences (SEQ ID NO: 1 and SEQ ID NO: 2). As described elsewhere herein, the listed amino acid sequences of the recombinant carbonic anhydrase polypeptides of SEQ ID NO: 4-100, which were expressed from E. coli, include an initiating methionine residue at position 1 (M1), whereas the listed amino acid sequences of the recombinant carbonic anhydrase polypeptides of SEQ ID NO: 120-302 do not include an initiating methionine residue at position 1 (M1). The listed recombinant carbonic anhydrase polypeptides of SEQ ID NO: 120-302 are provided as the polypeptides that were secreted from Bacillus megatarium, as such the initiating methionine (M1) which was part of the signal peptide construct, is cleaved and the first listed amino acid residue corresponds to position 2 of SEQ ID NO: 2 (e.g., Q2). It should be noted however, that due to the signal peptide construct used, each of the secreted carbonic anhydrase polypeptides of SEQ ID NO: 120-302 also has three amino acid residues Ala-Thr-Ser (encoded by a Spe1 restriction site) at the N-terminus. This N-terminus Ala-Thr-Ser is not included in the listed amino acid sequences of SEQ ID NO: 120-302.

TABLE 2
List of Sequences

| | Amino Acid Substitutions and |
|---|---|
| SEQ ID | Additional Amino and Carboxy Terminal Sequences |
| NO: | (As Compared To SEQ ID NO: 2) |
| 3/4 | E212K T213L S214H and the following 21 additional amino acids attached to |
| | the carboxy terminus: KAKLATITITIREEQMGKLDL (SEQ ID NO: 101) |
| 5/6 | S40V S58V E90K |
| 7/8 | S40V M56C S58V |
| 9/10 | M56H |
| 11/12 | S40V S58V |
| 13/14 | M56H S58V |
| 15/16 | M56H |
| 17/18 | S40V M56C S58V |
| 19/20 | M56H I87T |
| 21/22 | M56H E212G |
| 23/24 | D7S E212K T213L S214H and the following 21 additional amino acids |
| | attached to the carboxy terminus: KAKLATITITIREEQMGKLDL (SEQ ID NO: |
| | 101). |
| 25/26 | D7S T195M |
| 27/28 | D7S E23K G165N |
| 29/30 | D7S |
| 31/32 | D7S E95K D131N T195M |
| 33/34 | D7S T195M |
| 35/36 | D7S E95K T195M |
| 37/38 | D7S T195M |
| 39/40 | D7S D131N G165N T195M |
| 41/42 | D7S E95Q G165N T195M |
| 43/44 | D7S E95K D131N G165N T195M |
| 45/46 | D7S E95Q D131N G165N T195M |
| 47/48 | D7S D131N T195M |
| 49/50 | D7S D131N G165N E208V |
| 51/52 | D7S E95Q T195M |
| 53/54 | D7S D131N T195M |
| 55/56 | D7S E95K D131N G165N T195M |
| 57/58 | V122I |
| 59/60 | D7S E212K T213L S214H and the following 20 additional amino acids |
| | attached to the carboxy terminus: KAKLATITITIREEQMGKLD (SEQ ID NO: |
| | 102). |
| 61/62 | D7S E212K T213L S214H and the following 19 additional amino acids |
| | attached to the carboxy terminus: KAKLATITITIREEQMGKL (SEQ ID NO: |
| | 103). |
| 63/64 | D7S E212K T213L S214H and the following 18 additional amino acids |
| | attached to the carboxy terminus: KAKLATITITIREEQMGK (SEQ ID NO: |
| | 104). |
| 65/66 | D7S E212K T213L S214H and the following 17 additional amino acids |
| | attached to the carboxy terminus: KAKLATITITIREEQMG (SEQ ID NO: 105). |
| 67/68 | D7S E212K T213L S214H and the following 16 additional amino acids |
| | attached to the carboxy terminus: KAKLATITITIREEQM (SEQ ID NO: 106). |
| 69/70 | D7S E212K T213L S214H and the following 15 additional amino acids |
| | attached to the carboxy terminus: KAKLATITITIREEQ (SEQ ID NO: 107). |
| 71/72 | D7S E212K T213L S214H and the following 14 additional amino acids |
| | attached to the carboxy terminus: KAKLATITITIREE (SEQ ID NO: 108). |
| 73/74 | D7S E212K T213L S214H and the following 13 additional amino acids |
| | attached to the carboxy terminus: KAKLATITITIRE (SEQ ID NO: 109). |
| 75/76 | D7S E212K T213L S214H and the following 12 additional amino acids |
| | attached to the carboxy terminus: KAKLATITITIR (SEQ ID NO: 110). |
| 77/78 | D7S E212K T213L S214H and the following 11 additional amino acids |
| | attached to the carboxy terminus: KAKLATITITI (SEQ ID NO: 111). |
| 79/80 | D7S E212K T213L S214H and the following 10 additional amino acids |
| | attached to the carboxy terminus: KAKLATITIT (SEQ ID NO: 112). |
| 81/82 | D7S E212K T213L S214H and the following 9 additional amino acids attached |
| | to the carboxy terminus: KAKLATITI (SEQ ID NO: 113). |
| 83/84 | D7S E212K T213L S214H and the following 8 additional amino acids attached |
| | to the carboxy terminus: KAKLATIT (SEQ ID NO: 114). |
| 85/86 | D7S E212K T213L S214H and the following 7 additional amino acids attached |
| | to the carboxy terminus: KAKLATI (SEQ ID NO: 115). |
| 87/88 | D7S E212K T213L S214H and the following 6 additional amino acids attached |
| | to the carboxy terminus: KAKLAT (SEQ ID NO: 116). |
| 89/90 | D7S E212K T213L S214H and the following 5 additional amino acids attached |
| | to the carboxy terminus: KAKLA (SEQ ID NO: 117). |
| 91/92 | D7S E212K T213L S214H and the following 4 additional amino acids attached |
| | to the carboxy terminus: KAKL (SEQ ID NO: 118). |
| 93/94 | D7S E212K T213L S214H and the following 3 additional amino acids attached |
| | to the carboxy terminus: KAK |
| 95/96 | D7S E212K T213L S214H and the following 2 additional amino acids attached |
| | to the carboxy terminus: KA |
| 97/98 | D7S E212K T213L S214H and the following 1 additional amino acid attached |
| | to the carboxy terminus: K |
| 99/100 | D7S E212K T213L S214H |
| 119/120 | D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 121/122 | A191P; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 123/124 | N147A; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 125/126 | P16V; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 127/128 | A57V; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 129/130 | H194G; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 131/132 | A127R; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 133/134 | A26S; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 135/136 | E105W; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 137/138 | D7S; E212K; T213L; S214M; N-terminal ATS and M1 deleted; and the |
| | following 21 additional amino acids attached to the C-terminus: |
| | KAKLATITITIREEQMGKLDL (SEQ ID NO: 101). |
| 139/140 | T46L; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 141/142 | E3W; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 143/144 | A33G; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 145/146 | H194E; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 147/148 | E3A; P66G; D7S; E212K; T213L; S214H; N-terminal ATS and M1 deleted; and |
| | the following 21 additional amino acids attached to the C-terminus: |
| | KAKLATITITIREEQMGKLDL (SEQ ID NO: 101). |
| 149/150 | N147H; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 151/152 | P27L; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 153/154 | D7S; E212R; T213L; S214H; N-terminal ATS and M1 deleted; and the |
| | following 21 additional amino acids attached to the C-terminus: |
| | KAKLATITITIREEQMGKLDL (SEQ ID NO: 101). |
| 155/156 | Q2N; N11P; D7S; E212K; T213L; S214H; N-terminal ATS and M1 deleted; and |
| | the following 21 additional amino acids attached to the C-terminus: |
| | KAKLATITITIREEQMGKLDL (SEQ ID NO: 101). |
| 157/158 | C149S; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 159/160 | T161N; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 161/162 | E44A; A156T; D7S; E212K; T213L; S214H; N-terminal ATS and M1 deleted; |
| | and the following 21 additional amino acids attached to the C-terminus: |
| | KAKLATITITIREEQMGKLDL (SEQ ID NO: 101). |
| 163/164 | E44Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 165/166 | P27E; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 167/168 | D7S; E212K; T213L; S214E; N-terminal ATS and M1 deleted; and the |
| | following 21 additional amino acids attached to the C-terminus: |
| | KAKLATITITIREEQMGKLDL (SEQ ID NO: 101). |
| 169/170 | D36A; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 171/172 | D7S; E212K; T213L; S214W; N-terminal ATS and M1 deleted; and the |
| | following 21 additional amino acids attached to the C-terminus: |
| | KAKLATITITIREEQMGKLDL (SEQ ID NO: 101). |
| 173/174 | E3A; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 175/176 | V6M; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 177/178 | D7S; E212K; T213L; S214C; N-terminal ATS and M1 deleted; and the |
| | following 21 additional amino acids attached to the C-terminus: |
| | KAKLATITITIREEQMGKLDL (SEQ ID NO: 101). |
| 179/180 | P22K; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 181/182 | Q2P; T46S; D7S; E212K; T213L; S214H; N-terminal ATS and M1 deleted; and |
| | the following 21 additional amino acids attached to the C-terminus: |
| | KAKLATITITIREEQMGKLDL (SEQ ID NO: 101). |
| 183/184 | P31D; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 185/186 | K104Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 187/188 | E105T; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 189/190 | A138S; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 191/192 | E3L; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 193/194 | E14F; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 195/196 | V6Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 197/198 | D36H; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 199/200 | D7P; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 201/202 | Q2A; S10V; T46V; D7S; E212K; T213L; S214H; N-terminal ATS and M1 |
| | deleted; and the following 21 additional amino acids attached to the C-terminus: |
| | KAKLATITITIREEQMGKLDL (SEQ ID NO: 101). |
| 203/204 | E8A; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 205/206 | S40C; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 207/208 | Q137G; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 209/210 | G165K; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 211/212 | T46D; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 213/214 | D7S; E212K; T213L; S214D; N-terminal ATS and M1 deleted; and the |
| | following 21 additional amino acids attached to the C-terminus: |
| | KAKLATITITIREEQMGKLDL (SEQ ID NO: 101). |
| 215/216 | Q2H; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 217/218 | S10W; P37H; D7S; E212K; T213L; S214H; N-terminal ATS and M1 deleted; |
| | and the following 21 additional amino acids attached to the C-terminus: |
| | KAKLATITITIREEQMGKLDL (SEQ ID NO: 101). |
| 219/220 | A127E; D7S; E212K; T213L; S214K; N-terminal ATS and M1 deleted; and the |
| | following 21 additional amino acids attached to the C-terminus: |
| | KAKLATITITIREEQMGKLDL (SEQ ID NO: 101). |
| 221/222 | E23G; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 223/224 | H194A; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 225/226 | E235; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 227/228 | P31Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 229/230 | N203I; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 231/232 | E44P; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 233/234 | P31C; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 235/236 | E8Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 237/238 | A127W; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 239/240 | K142Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 241/242 | P22I; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 243/244 | I98V; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 245/246 | I98K; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 247/248 | M136Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 249/250 | F139M; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 251/252 | F139V; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 253/254 | V204T; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 255/256 | V204Q; D7S; E212K; T213L; S214H; N-terminal ATS and M1 deleted; and the |
| | following 21 additional amino acids attached to the C-terminus: |
| | KAKLATITITIREEQMGKLDL (SEQ ID NO: 101). |
| 257/258 | D7S; E212K; T213L; S214H; N-terminal ATS and M1 deleted; and the |
| | following 21 additional amino acids attached to the C-terminus: |
| | KAKLATITITIPEEQMGKLDL (SEQ ID NO: 318). (R226P) |
| 259/260 | D7S; E212K; T213L; S214H; N-terminal ATS and M1 deleted; and the |
| | following 21 additional amino acids attached to the C-terminus: |
| | KAKLATIGITIREEQMGKLDL (SEQ ID NO: 319). (T222G) |
| 261/262 | D7S; E212K; T213L; S214H; N-terminal ATS and M1 deleted; and the |
| | following 21 additional amino acids attached to the C-terminus: |
| | KAKLATITITIDEEQMGKLDL (SEQ ID NO: 320). (R226D) |
| 263/264 | D7S; E212K; T213L; S214H; N-terminal ATS and M1 deleted; and the |
| | following 21 additional amino acids attached to the C-terminus: |
| | KAKLATITITIREEQMGKLDT (SEQ ID NO: 321). (L235T) |
| 265/266 | D7S; E212K; T213L; S214H; N-terminal ATS and M1 deleted; and the |
| | following 21 additional amino acids attached to the C-terminus: |
| | KAKLATITITIREEQMGKLDV (SEQ ID NO: 322). (L235V) |
| 267/268 | D7S; E212K; T213L; S214H; N-terminal ATS and M1 deleted; and the |
| | following 21 additional amino acids attached to the C-terminus: |
| | KAKLATITITIREEQMGKLDS (SEQ ID NO: 323). (L235S) |
| 269/270 | D7S; E212K; T213L; S214H; N-terminal ATS and M1 deleted; and the |
| | following 21 additional amino acids attached to the C-terminus: |
| | KAKLATITITMREEQMGKLDL (SEQ ID NO: 324). (I225M) |
| 271/272 | D7S; E212K; T213L; S214H; N-terminal ATS and M1 deleted; and the |
| | following 21 additional amino acids attached to the C-terminus: |
| | KAKLATITITIREEQAGKLDL (SEQ ID NO: 325). (M230A) |
| 273/274 | D7S; E212K; T213L; S214H; N-terminal ATS and M1 deleted; and the |
| | following 21 additional amino acids attached to the C-terminus: |
| | KSKLATITITIREEQMGKLDL (SEQ ID NO: 326). (A216S) |
| 275/276 | D7S; E212K; T213L; S214H; N-terminal ATS and M1 deleted; and the |
| | following 21 additional amino acids attached to the C-terminus: |
| | KAKLAGITITIREEQMGKLDL (SEQ ID NO: 327). (T220G) |
| 277/278 | D7S; E212K; T213L; S214H; N-terminal ATS and M1 deleted; and the |
| | following 21 additional amino acids attached to the C-terminus: |
| | AAKLATITITIREEQMGKLDL (SEQ ID NO: 328). (K215A) |
| 279/280 | D7S; E212K; T213L; S214H; N-terminal ATS and M1 deleted; and the |
| | following 21 additional amino acids attached to the C-terminus: |
| | KAKLATIEITIREEQMGKLDL (SEQ ID NO: 329). (T222E) |
| 281/282 | D7S; E212K; T213L; S214H; N-terminal ATS and M1 deleted; and the |
| | following 21 additional amino acids attached to the C-terminus: |
| | KAKLATITITLREEQMGKLDL (SEQ ID NO: 330). (I225L) |
| 283/284 | D7S; E212K; T213L; S214H; N-terminal ATS and M1 deleted; and the |
| | following 21 additional amino acids attached to the C-terminus: |
| | KAKLANITITIREEQMGKLDL (SEQ ID NO: 331). (T220N) |
| 285/286 | D7S; E212K; T213L; S214H; N-terminal ATS and M1 deleted; and the |
| | following 21 additional amino acids attached to the C-terminus: |
| | KAKLATITITIGEEQMGKLDL (SEQ ID NO: 332). (R226G) |
| 287/288 | D7S; E212K; T213L; S214H; N-terminal ATS and M1 deleted; and the |
| | following 21 additional amino acids attached to the C-terminus: |
| | KAKLATITITIREEQMDKLDL (SEQ ID NO: 333). (G231D) |
| 289/290 | D7S; E212K; T213L; S214H; N-terminal ATS and M1 deleted; and the |
| | following 21 additional amino acids attached to the C-terminus: |
| | KAKLATITITIREEQMGKQDL (SEQ ID NO: 334). (L233Q) |
| 291/292 | D7S; E212K; T213L; S214H; N-terminal ATS and M1 deleted; and the |
| | following 21 additional amino acids attached to the C-terminus: |
| | KAGLATITITIREEQMGKLDL (SEQ ID NO: 335). (K217G) |
| 293/294 | D7S; E212K; T213L; S214H; N-terminal ATS and M1 deleted; and the |
| | following 21 additional amino acids attached to the C-terminus: |
| | KAKLATITITCREEQMGKLDL (SEQ ID NO: 336). (I225C) |
| 295/296 | D7S; E212K; T213L; S214H; N-terminal ATS and M1 deleted; and the |
| | following 21 additional amino acids attached to the C-terminus: |
| | KAKLATGTITIREEQMGKLDL (SEQ ID NO: 337). (I221G) |
| 297/298 | D7S; E212K; T213L; S214H; N-terminal ATS and M1 deleted; and the |
| | following 21 additional amino acids attached to the C-terminus: |
| | KAKLATITTTIREEQMGKLDL (SEQ ID NO: 338). (I223T) |
| 299/300 | D7S; E212K; T213L; S214H; N-terminal ATS and M1 deleted; and the |
| | following 21 additional amino acids attached to the C-terminus: |
| | KAKLADITITIREEQMGKLDL (SEQ ID NO: 339). (T220D) |
| 301/302 | D7S; E212K; T213L; S214H; N-terminal ATS and M1 deleted; and the |
| | following 21 additional amino acids attached to the C-terminus: |
| | KAKLATITITGREEQMGKLDL (SEQ ID NO: 340). (I225G) |

In some embodiments, the present disclosure provides improved recombinant carbonic anhydrase polypeptides comprising an amino acid sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical as compared to SEQ ID NO:2 and comprises at least one amino acid residue difference (e.g., substitution, insertion, and/or deletion) listed in Table 2. Such improved carbonic anhydrase polypeptides disclosed herein may further comprise additional modifications, including substitutions, deletions, insertions, or combinations thereof. The substitutions can be non-conservative substitutions, conservative substitutions, or a combination of non-conservative and conservative substitutions. In some embodiments, these carbonic anhydrase polypeptides can have optionally from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 mutations at other amino acid residues. In some embodiments, the number of modifications can be 1, 2, 3, 4, 5, 6, 7, 8, 9, 10, 11, 12, 14, 15, 16, 18, 20, 22, 24, 26, 30, 35 or about 40 other amino acid residues.

In certain embodiments, the present disclosure provides recombinant carbonic anhydrase polypeptides having an improved enzyme property relative to the reference sequence of SEQ ID NO:2, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO:2, and at least one of the amino acid substitutions listed in Table 2 at a position corresponding to any one of the position 2 to position 214 of SEQ ID NO:2.

In certain embodiments, the present disclosure provides a recombinant carbonic anhydrase polypeptide having an improved enzyme property relative to the reference sequence of SEQ ID NO:2, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO:2, and at least one of the following amino acid substitutions at the position corresponding to the indicated position of SEQ ID NO:2: residue at position 2 is an aliphatic or non-polar amino acid selected from the group consisting of alanine, leucine, isoleucine, valine, glycine, and methionine, or a polar amino acid selected from the group consisting of asparagine, serine, and threonine, or a constrained amino acid selected from the group consisting of proline and histidine; residue at position 3 is an aliphatic or non-polar amino acid selected from the group consisting of alanine, leucine, isoleucine, valine, glycine, and methionine, or an aromatic amino acid selected from phenylalanine, tyrosine, or tryptophan; residue at position 6 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 7 is a polar amino acid selected from the group consisting of asparagine, glutamine, serine, and threonine, or a constrained amino acid selected from the group consisting of proline and histidine; residue at position 8 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 10 is an aliphatic or non-polar amino acid selected from the group consisting of alanine, leucine, isoleucine, valine, glycine, and methionine, or an aromatic amino acid selected from phenylalanine, tyrosine, or tryptophan; residue at position 11 is a constrained amino acid selected from the group consisting of proline and histidine; residue at position 14 is an aromatic amino acid selected from phenylalanine, tyrosine, or tryptophan; residue at position 16 is an aliphatic or non-polar amino acid selected from the group consisting of alanine, leucine, isoleucine, valine, glycine, and methionine; residue at position 22 is an aliphatic or non-polar amino acid selected from the group consisting of alanine, leucine, isoleucine, valine, glycine, and methionine, or a basic amino acid selected from the group consisting of lysine and arginine; residue at position 23 is a basic amino selected from the group consisting of lysine and arginine, or a non-polar amino acid selected from the group consisting of alanine, leucine, isoleucine, valine, glycine, and methionine, or a polar amino acid selected from the group consisting of asparagine, glutamine, serine, and threonine; residue at position 26 is a polar amino acid selected from the group consisting of asparagine, glutamine, serine, and threonine; residue at position 27 is a non-polar amino acid selected from the group consisting of alanine, leucine, isoleucine, valine, glycine, and methionine, or an acidic amino acid selected from aspartic acid and glutamic acid; residue at position 31 is a cysteine, or an acidic amino acid selected from aspartic acid and glutamic acid, or a polar amino acid selected from the group consisting of asparagine, glutamine, serine, and threonine; residue at position 33 is an aliphatic or non-polar amino acid selected from the group consisting of alanine, leucine, isoleucine, valine, glycine, and methionine; residue at position 36 is an aliphatic or non-polar amino acid selected from the group consisting of alanine, leucine, isoleucine, valine, glycine, and methionine, or a constrained amino acid selected from the group consisting of proline and histidine; residue at position 37 is a constrained amino acid selected from the group consisting of proline and histidine; residue at position 40 is an aliphatic or non-polar amino acid selected from the group consisting of alanine, leucine, isoleucine, valine, glycine, and methionine, or a cysteine; residue at position 44 is an aliphatic or non-polar amino acid selected from the group consisting of alanine, leucine, isoleucine, valine, glycine, and methionine, or a polar amino acid selected from the group consisting of asparagine, glutamine, serine, and threonine, or a constrained amino acid selected from the group consisting of proline and histidine; residue at position 46 is an aliphatic or non-polar amino acid selected from the group consisting of alanine, leucine, isoleucine, valine, glycine, and methionine, or a polar amino acid selected from the group consisting of asparagine, glutamine, and serine, or an acidic amino acid selected from aspartic acid and glutamic acid; residue at position 56 is cysteine or a constrained amino acid selected from the group consisting of proline and histidine; residue at position 57 is an aliphatic or non-polar amino acid selected from the group consisting of alanine, leucine, isoleucine, valine, glycine, and methionine; residue at position 58 is an aliphatic or non-polar amino acid selected from the group consisting of alanine, leucine, isoleucine, valine, glycine, and methionine; residue at position 87 is a polar amino acid selected from the group consisting of asparagine, glutamine, serine, and threonine; residue at position 90 is a basic amino acid selected from the group consisting of lysine and arginine; residue at position 95 is a polar amino acid selected from the group consisting of asparagine, glutamine, serine, and threonine, or a basic amino acid selected from the group consisting of lysine and arginine; residue at position 98 is an aliphatic or non-polar amino acid selected from the group consisting of alanine, leucine, valine, glycine, and methionine, or a basic amino acid selected from the group consisting of lysine and arginine; residue at position 104 is a polar amino acid selected from the group consisting of asparagine, glutamine, serine, and threonine; residue at position 105 is a polar amino acid selected from the group consisting of asparagine, glutamine, serine, and threonine, or an aromatic amino acid selected from phenylalanine, tyrosine, or tryptophan; residue at position 122 is an aliphatic or non-polar amino acid selected from the group consisting of alanine, leucine, isoleucine, glycine, and methionine; residue at position 127 is an acidic amino acid selected from aspartic acid and glutamic acid, or a basic amino acid selected from the group consisting of lysine and arginine, or an aromatic amino acid selected from phenylalanine, tyrosine, or tryptophan; residue at position 131 is a polar amino acid selected from the group consisting of asparagine, glutamine, serine, and threonine; residue at position 136 is a polar amino acid selected from the group consisting of asparagine, glutamine, serine, and threonine; residue at position 137 is an aliphatic or non-polar amino acid selected from the group consisting of alanine, leucine, isoleucine, valine, glycine, and methionine; residue at position 138 is a polar amino acid selected from the group consisting of asparagine, glutamine, serine, and threonine; residue at position 139 is an aliphatic or non-polar amino acid selected from the group consisting of alanine, leucine, isoleucine, valine, glycine, and methionine; residue at position 142 is a polar amino acid selected from the group consisting of asparagine, glutamine, serine, and threonine; residue at position 147 is a polar amino acid selected from the group consisting of asparagine, glutamine, serine, and threonine, or a constrained amino acid selected from the group consisting of proline and histidine; residue at position 149 is a polar amino acid selected from the group consisting of asparagine, glutamine, serine, and threonine; residue at position 156 is a polar amino acid selected from the group consisting of asparagine, glutamine, serine, and threonine; residue at position 161 is a polar amino acid selected from the group consisting of asparagine, glutamine, or serine; residue at position 165 is a polar amino acid selected from the group consisting of asparagine, glutamine, serine, and threonine, or a basic amino acid selected from the group consisting of lysine and arginine; residue at position 191 is a constrained amino acid selected from the group consisting of proline and histidine; residue at position 194 is an aliphatic or non-polar amino acid selected from the group consisting of alanine, leucine, isoleucine, valine, glycine, and methionine, or an acidic amino acid selected from aspartic acid and glutamic acid; residue at position 195 is a non-polar amino acid selected from the group consisting of alanine, leucine, isoleucine, valine, glycine, and methionine; residue at position 203 is an aliphatic or non-polar amino acid selected from the group consisting of alanine, leucine, isoleucine, valine, glycine, and methionine; residue at position 204 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 208 is an aliphatic or non-polar amino acid selected from the group consisting of alanine, leucine, isoleucine, valine, glycine, and methionine; residue at position 212 is a basic amino acid selected from the group consisting of arginine and lysine, or a non-polar amino acid selected from the group consisting of alanine, leucine, isoleucine, valine, glycine, and methionine; residue at position 213 is an aliphatic or non-polar amino acid selected from the group consisting of alanine, leucine, isoleucine, valine, glycine, and methionine; and residue at position 214 is a cysteine, or an acidic amino acid selected from aspartic acid and glutamic acid, or an aliphatic or non-polar amino acid selected from the group consisting of alanine, leucine, isoleucine, valine, glycine, and methionine, or a basic amino acid selected from the group consisting of lysine and arginine, or an aromatic amino acid selected from phenylalanine, tyrosine, or tryptophan, or a constrained amino acid selected from the group consisting of proline and histidine. Such improved recombinant carbonic anhydrase polypeptides disclosed herein may further comprise additional modifications, including substitutions, deletions, insertions, or combinations thereof. The substitutions can be non-conservative substitutions, conservative substitutions, or a combination of non-conservative and conservative substitutions. In some embodiments, these carbonic anhydrase polypeptides can have optionally from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 mutations at other amino acid residues. In some embodiments, the number of modifications can be 1, 2, 3, 4, 5, 6, 7, 8, 9, 10, 11, 12, 14, 15, 16, 18, 20, 22, 24, 26, 30, 35 or about 40 other amino acid residues.

In certain embodiments, the present disclosure provides a recombinant carbonic anhydrase polypeptide having an improved enzyme property relative to the reference sequence of SEQ ID NO:2, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO:2, and at least one of the following amino acid substitutions at the position corresponding to the indicated position of SEQ ID NO:2: residue at position 2 is alanine, histidine, asparagine, or proline; residue at position 3 is alanine, leucine, or tryptophan; residue at position 6 is methionine, or glutamine; residue at position 7 is proline, or serine; residue at position 8 is alanine, or glutamine; residue at position 10 is valine, or tryptophan; residue at position 11 is proline; residue at position 14 is phenylalanine; residue at position 16 is valine; residue at position 22 is isoleucine, or lysine; residue at position 23 is glycine, lysine, or serine; residue at position 26 is serine; residue at position 27 is glutamic acid, or leucine; residue at position 31 is cysteine, aspartic acid, or glutamine; residue at position 33 is glycine; residue at position 36 is alanine, or histidine; residue at position 37 is histidine; residue at position 40 is cysteine, or valine; residue at position 44 is alanine, proline, or glutamine; residue at position 46 is aspartic acid, leucine, serine, or valine; residue at position 56 is cysteine, or histidine; residue at position 57 is valine; residue at position 58 is valine; residue at position 87 is threonine; residue at position 90 is lysine; residue at position 95 is glutamine; residue at position 98 is lysine, or valine; residue at position 104 is glutamine; residue at position 105 is threonine, or tryptophan; residue at position 122 is isoleucine; residue at position 127 is glutamic acid, arginine, or tryptophan; residue at position 131 is asparagine; residue at position 136 is glutamine; residue at position 137 is glycine; residue at position 138 is serine; residue at position 139 is methionine, or valine; residue at position 142 is glutamine; residue at position 147 is alanine, or histidine; residue at position 149 is serine; residue at position 156 is threonine; residue at position 161 is asparagine; residue at position 165 is asparagine, or lysine; residue at position 191 is proline; residue at position 194 is alanine, glutamic acid, or glycine; residue at position 195 is methionine; residue at position 203 is isoleucine; residue at position 204 is glycine, glutamine, or threonine; residue at position 208 is valine; residue at position 212 is arginine, glycine, or lysine; residue at position 213 is leucine; and residue at position 214 is cysteine, aspartic acid, glutamic acid, histidine, lysine, methionine, or tryptophan.

In certain embodiments, the present disclosure provides a recombinant carbonic anhydrase polypeptide having an improved enzyme property relative to a reference polypeptide of SEQ ID NO:2, wherein said polypeptide comprises an amino acid sequence having at least 80% identity to SEQ ID NO:2 and one or more of the following amino acid substitutions at the position corresponding to the indicated position of SEQ ID NO: 2: residue at position 2 is alanine, histidine, asparagine, or proline; residue at position 3 is tryptophan; residue at position 7 is proline; residue at position 8 is alanine, or glutamine; residue at position 10 is valine, or tryptophan; residue at position 11 is proline; residue at position 14 is phenylalanine; residue at position 16 is valine; residue at position 22 is isoleucine, or lysine; residue at position 23 is lysine, or serine; residue at position 26 is serine; residue at position 27 is glutamic acid, or leucine; residue at position 31 is cysteine, or aspartic acid; residue at position 33 is glycine; residue at position 36 is alanine; residue at position 37 is histidine; residue at position 40 is cysteine; residue at position 46 is aspartic acid, leucine, serine, or valine; residue at position 56 is cysteine, or histidine; residue at position 57 is valine; residue at position 58 is valine; residue at position 87 is threonine; residue at position 90 is lysine; residue at position 95 is glutamine; residue at position 98 is lysine; residue at position 105 is threonine, or tryptophan; residue at position 127 is glutamic acid, or arginine; residue at position 131 is asparagine; residue at position 136 is glutamine; residue at position 137 is glycine; residue at position 142 is glutamine; residue at position 147 is alanine, or histidine; residue at position 149 is serine; residue at position 156 is threonine; residue at position 161 is asparagine; residue at position 165 is asparagine, or lysine; residue at position 191 is proline; residue at position 194 is alanine, glutamic acid, or glycine; residue at position 195 is methionine; residue at position 203 is isoleucine; residue at position 212 is glycine; residue at position 213 is leucine; residue at position 214 is cysteine, aspartic acid, glutamic acid, histidine, lysine, methionine, or tryptophan.

In certain embodiments, the recombinant carbonic anhydrase polypeptide having an improved enzyme property relative to a reference polypeptide of SEQ ID NO:2, an amino acid sequence having at least 80% identity to SEQ ID NO:2, and one or more of the above-listed amino acid substitutions, additionally comprises one or more of the following amino acid substitutions at the position corresponding to the indicated position of SEQ ID NO: 2: residue at position 3 is alanine, leucine, or tryptophan; residue at position 6 is methionine, or glutamine; residue at position 7 is proline, or serine; residue at position 23 is glycine, lysine, or serine; residue at position 31 is cysteine, aspartic acid, or glutamine; residue at position 36 is alanine, or histidine; residue at position 40 is cysteine, or valine; residue at position 44 is alanine, proline, or glutamine; residue at position 98 is lysine, or valine; residue at position 104 is glutamine; residue at position 105 is threonine, or tryptophan; residue at position 122 is isoleucine; residue at position 127 is glutamic acid, arginine, or tryptophan; residue at position 138 is serine; residue at position 139 is methionine, or valine; residue at position 204 is glycine, glutamine, or threonine; residue at position 208 is valine; residue at position 212 is arginine, glycine, or lysine.

In certain embodiments, the recombinant carbonic anhydrase polypeptide having an improved enzyme property relative to a reference polypeptide of SEQ ID NO:2, an amino acid sequence having at least 80% identity to SEQ ID NO:2, and one or more of the above-listed amino acid substitutions, additionally comprises one or more of the following amino acid substitutions at the position corresponding to the indicated position of SEQ ID NO: 2: residue at position 7 is proline, or serine; residue at position 212 is arginine, glycine, or lysine.

In certain embodiments, the recombinant carbonic anhydrase polypeptide having an improved enzyme property relative to a reference polypeptide of SEQ ID NO:2, an amino acid sequence having at least 80% identity to SEQ ID NO:2, and one or more of the above-listed amino acid substitutions, additionally comprises at least two of the following amino acid substitutions at the position corresponding to the indicated position of SEQ ID NO: 2: residue at position 7 is proline, or serine; residue at position 212 is arginine, glycine, or lysine; residue at position 213 is leucine; residue at position 214 is cysteine, aspartic acid, glutamic acid, histidine, lysine, methionine, or tryptophan.

In some embodiments, the recombinant carbonic anhydrase polypeptide of the present disclosure having an improved enzyme property relative to a reference polypeptide of SEQ ID NO:2, an amino acid sequence having at least 80% identity to SEQ ID NO:2, comprises the following at least three of the following four amino acid substitutions at the position corresponding to the indicated position of SEQ ID NO: 2: residue at position 7 is serine; residue at position 212 is lysine; residue at position 213 is leucine; and residue at position 214 is histidine. In some embodiments, the recombinant carbonic anhydrase polypeptide comprises all four of the amino acid substitutions: residue at position 7 is serine; residue at position 212 is lysine; residue at position 213 is leucine; and residue at position 214 is histidine

In certain embodiments, the present disclosure provides a recombinant carbonic anhydrase polypeptide having an improved enzyme property relative to a reference polypeptide of SEQ ID NO:2, an amino acid sequence having at least 80% identity to SEQ ID NO:2, wherein the amino acid sequence comprises one or more of the following amino acid substitutions at the position corresponding to the indicated position of SEQ ID NO: 2: Q2A; Q2H; Q2N; Q2P; E3A; E3L; E3W; V6M; V6Q; D7P; D7S; E8A; E8Q; S10V; S10W; N11P; E14F; P16V; P22I; P22K; E23G; E23K; E23S; A26S; P27E; P27L; P31C; P31D; P31Q; A33G; D36A; D36H; P37H; S40C; S40V; E44A; E44P; E44Q; T46D; T46L; T46S; T46V; M56C; M56H; A57V; S58V; P66G; I87T; E90K; E95K; E95Q; I98K; I98V; K104Q; E105T; E105W; V122I; A127E; A127R; A127W; D131N; M136Q; Q137G; A138S; F139M; F139V; K142Q; N147A; N147H; C149S; A156T; T161N; G165K; G165N; A191P; H194A; H194E; H194G; T195M; N203I; V204Q; V204T; E208V; E212G; E212K; E212R; T213L; S214C; S214D; S214E; S214H; S214K; S214M; S214W.

In certain embodiments, the disclosure provides a recombinant carbonic anhydrase polypeptide having an improved enzyme property relative to a reference polypeptide of SEQ ID NO:2 which comprises an amino acid sequence selected from the group consisting of SEQ ID NO: 4, 6, 10, 12, 14, 16, 20, 22, 24, 28, 36, 38, 44, 50, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

As described herein, the carbonic anhydrase polypeptides of the disclosure can be in the form of fusion polypeptides in which the carbonic anhydrase polypeptides are fused to other polypeptides, such as antibody tags (e.g., myc epitope) or purifications sequences (e.g., His tags). Thus, the carbonic anhydrase polypeptides can be used with or without fusions to other polypeptides.

In certain embodiments, the recombinant carbonic anhydrase polypeptides of the present disclosure further comprise additional amino acids at the amino terminus and/or the carboxyl terminus. In some embodiments, the recombinant carbonic anhydrase polypeptides further comprise a carboxy terminal fusion of from about 5 to about 40, from about 10 to about 30, or about 20 additional amino acids at the carboxyl terminus. In some embodiments, the carboxy terminal fusion comprises an additional 21 amino acids beginning after the residue corresponding to 5214 of SEQ ID NO: 2.

In some embodiments, a recombinant carbonic anhydrase polypeptide of the present disclosure further comprises a fusion polypeptide at its carboxy terminus of any one of SEQ ID NOs: 101-118. For example, the carbonic anhydrase polypeptides of SEQ ID NOs: 4 and 24 each comprises a 21 amino acid C-terminal fusion of SEQ ID NO: 101. It has been observed that the polypeptides of SEQ ID NOs: 101-118 when attached as a fusion polypeptide to the C-terminus carbonic anhydrase polypeptide results in increased thermostability relative to the carbonic anhydrase without the extension polypeptide. As described further in Example 9, the carbonic anhydrase polypeptides of SEQ ID NOs: 24, 60, 62, 64, 66, 68, 70, 72, 74, 76, 78, 80, 82, 84, 86, 88, 90, and 92, each comprises a C-terminal extension polypeptide of SEQ ID NOs: 101, 102, 103, 104, 105, 106, 107, 108, 109, 110, 111, 112, 113, 114, 115, 116, 117, and 118, respectively. Each of these carbonic anhydrase polypeptides with C-terminal fusion exhibits increased thermostability relative to the polypeptide without any C-terminal fusion (e.g., SEQ ID NO: 100, which corresponds to the polypeptide of SEQ ID NO: 24 without the 21 amino acid fusion of SEQ ID NO: 101).

Additionally, the carbonic anhydrase polypeptides of SEQ ID NOs: 94, 96, and 98, each comprises a short (less than 4 amino acid) C-terminal extension of Lys-Ala-Lys, Lys-Ala, and Lys, respectively, and yet still exhibit increased thermostability relative to the polypeptide without any C-terminal fusion. Thus, in some embodiments the carbonic anhydrase polypeptides can comprise short C-terminal fusions of Lys-Ala-Lys, Lys-Ala, or just a Lys amino acid.

Similarly, in some embodiments the present disclosure contemplates a recombinant carbonic anhydrase polypeptide wherein the amino acid sequence further comprises a fusion polypeptide at its carboxy terminus of any one of SEQ ID NOs: 316-338. For example, the carbonic anhydrase polypeptides of SEQ ID NOs: 258, 260, 262, 264, 266, 268, 270, 272, 274, 276, 278, 280, 282, 284, 286, 288, 290, 292, 294, 296, 298, 300, and 302 each comprises a 21 amino acid C-terminal fusion of SEQ ID NOs: 316-338, respectively. Each of the polypeptides of SEQ ID NOs: 316-338 includes an amino acid substitution relative to the 21 amino acid C-terminal fusion of SEQ ID NO: 101. As described further in Example 11, the substituted C-terminal extension polypeptides of SEQ ID NOs: 316-338 results in increased carbonic anhydrase activity under basic conditions (1.5 M AMP co-solvent, pH 9.7) —i.e., increased base stability, relative to SEQ ID NO: 120, which has the extension of SEQ ID NO: 101.

Accordingly, it is contemplated in some embodiments that the C-terminal extension (or fusion) polypeptides represented by SEQ ID NOs: 101-118, 316-338, or Lys-Ala-Lys, Lys-Ala, or just a Lys amino acid, can be used with any carbonic anhydrase polypeptide that does not already include such an extension (e.g., SEQ ID NOs: 2, 6, 8, 10, 12, 14, 16, 18, 20, 22, 26, 28, 30, 32, 34, 36, 38, 40, 42, 44, 46, 48, 50, 52, 54, 56, 58, and 100) to provide a carbonic anhydrase having the improved property of increased thermostability and/or increased basic solvent stability. Thus, in some embodiments the present disclosure provides a carbonic anhydrase polypeptide comprising a C-terminal extension (i.e., at position 314) of any one of SEQ ID NOs: 101-118, 316-338, or a Lys-Ala-Lys, Lys-Ala, or just a Lys amino acid, wherein the carbonic anhydrase has increased thermostability relative to the carbonic anhydrase without the C-terminal extension.

In certain embodiments, the present disclosure provides a recombinant carbonic anhydrase polypeptide having an improved enzyme property relative to a reference sequence of SEQ ID NO:2, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:2, and wherein the amino acid sequence further comprises a carboxy terminal fusion of any one of the polypeptides of SEQ ID NOs: 101-118, 316-338, KAK, KA, or the single amino acid K. In some embodiments, the amino acid sequence further comprises a carboxy terminal fusion of a polypeptide of SEQ ID NO: 101.

In some embodiments the present disclosure provides a recombinant carbonic anhydrase polypeptide having an improved enzyme property relative to a reference sequence of SEQ ID NO:2, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO:2, wherein the amino acid sequence comprises one or more of the amino acid substitutions listed in Table 2 at the position corresponding to the indicated position of a polypeptide comprising SEQ ID NO: 2 and the carboxy terminal fusion of a polypeptide of SEQ ID NO: 101.

Accordingly, in some embodiments, the recombinant carbonic anhydrase polypeptide comprises the amino acid sequence of SEQ ID NO: 2 and the carboxy terminal fusion of a polypeptide of SEQ ID NO: 101, wherein the sequence comprises at least one substitution selected from: Q2A; Q2H; Q2N; Q2P; E3A; E3L; E3W; V6M; V6Q; D7P; D7S; E8A; E8Q; 510V; 510W; N11P; E14F; P16V; P22I; P22K; E23G; E23K; E23S; A26S; P27E; P27L; P31C; P31D; P31Q; A33G; D36A; D36H; P37H; 540C; 540V; E44A; E44P; E44Q; T46D; T46L; T46S; T46V; M56C; M56H; A57V; S58V; P66G; I87T; E90K; E95K; E95Q; I98K; I98V; K104Q; E105T; E105W; V122I; A127E; A127R; A127W; D131N; M136Q; Q137G; A138S; F139M; F139V; K142Q; N147A; N147H; C149S; A156T; T161N; G165K; G165N; A191P; H194A; H194E; H194G; T195M; N203I; V204Q; V204T; E208V; E212G; E212K; E212R; T213L; S214C; S214D; S214E; S214H; S214K; S214M; S214W; K215A; A216S; K217G; T220D; T220G; T220N; I221G; T222E; T222G; I223T; I225C; I225G; I225L; I225M; R226D; R226G; R226P; M230A; G231D; L233Q; L235S; L235T; L235V.

In certain embodiments, the present disclosure provides a recombinant carbonic anhydrase polypeptide having an improved enzyme property relative to a reference sequence of SEQ ID NO:24 or SEQ ID NO: 120, wherein the polypeptide comprises an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO:24 or SEQ ID NO: 120, and comprises at least one of the amino acid substitutions listed in Table 2 at a position corresponding to any one of the position 2 to position 235 of SEQ ID NO:24 or SEQ ID NO:120.

In some embodiments, the recombinant carbonic anhydrase polypeptide having an improved enzyme property relative to a reference sequence of SEQ ID NO:120, wherein the polypeptide comprises an amino acid sequence at least about 80% identical to SEQ ID NO: 120 with one or more of the following amino acid substitutions at the position corresponding to the indicated position of SEQ ID NO: 2: Q2A; Q2H; Q2N; Q2P; E3A; E3L; E3W; V6M; V6Q; S7P; E8A; E8Q; S10V; S10W; N11P; E14F; P16V; P22I; P22K; E23G; E23S; A26S; P27E; P27L; P31C; P31D; P31Q; A33G; D36A; D36H; P37H; 540C; E44A; E44P; E44Q; T46D; T46L; T46S; T46V; A57V; P66G; I98K; I98V; K104Q; E105T; E105W; A127E; A127R; A127W; M136Q; Q137G; A138S; F139M; F139V; K142Q; N147A; N147H; C149S; A156T; T161N; G165K; A191P; H194A; H194E; H194G; N203I; V204Q; V204T; K212R; H214C; H214D; H214E; H214K; H214M; H214W; K215A; A216S; K217G; T220D; T220G; T220N; I221G; T222E; T222G; I223T; I225C; I225G; I225L; I225M; R226D; R226G; R226P; M230A; G231D; L233Q; L235S; L235T; L235V.

In some embodiments, the recombinant carbonic anhydrase polypeptide having an improved enzyme property relative to a reference sequence of SEQ ID NO:120, wherein the polypeptide comprises an amino acid sequence at least about 80% identical to SEQ ID NO: 120 with one or more of the following amino acid substitutions at the position corresponding to the indicated position of SEQ ID NO: 2: Q2A; Q2H; Q2N; Q2P; E3A; E3L; E3W; V6Q; S7P; E8A; S10V; S10W; N11P; E14F; P22I; P22K; E23S; A26S; P31C; P31Q; A33G; D36H; P37H; 540C; E44P; E44Q; T46D; T46L; T46S; T46V; A57V; P66G; I98K; E105T; E105W; A127E; A127R; A127W; Q137G; A138S; F139M; K142Q; N147A; T161N; G165K; H194A; H194E; N203I; V204Q; V204T; K212R; H214C; H214D; H214E; H214K; H214M; K215A; T220D; T220G; T220N; T222E; I223T; I225L; R226D; R226G; R226P; G231D; L235S; L235T; and L235V.

In some embodiments, the recombinant carbonic anhydrase polypeptide having an improved enzyme property relative to a reference sequence of SEQ ID NO:120, wherein the polypeptide comprises an amino acid sequence at least about 80% identical to SEQ ID NO: 120 with one or more of the following amino acid substitutions at the position corresponding to the indicated position of SEQ ID NO: 2: Q2P; E3L; E3W; S7P; E14F; P22K; A26S; P31C; A33G; D36H; E44P; E44Q; T46D; T46L; T46S; A127E; A127R; Q137G; A138S; F139M; T161N; N203I; H214D; H214E; H214K; H214M; T220D; I225L; R226G; and L235T.

In some embodiments, the amino acid sequence of a recombinant carbonic anhydrase polypeptide as disclosed herein can further comprise a signal peptide sequence, whereby the polypeptide is secreted by a host cell. In some embodiments, the recombinant carbonic anhydrase polypeptide comprises a signal peptide sequence a selected from SEQ ID NO: 313, 314, and 315.

The ordinary artisan will recognize that in embodiments involving signal peptides, the methionine codon at position 1 will be deleted in the polynucleotides encoding the recombinant carbonic anhydrase polypeptides of SEQ ID NO: 4, 6, 10, 12, 14, 16, 20, 22, 24, 28, 36, 38, 44, 50, 56, 58, 60, 62, 64, 66, 68, 70, 72, 74, 76, 78, 80, 82, 84, 86, 88, 90, 92, 94, 96, 98, 100.

Suitable host cells and signal peptides useful for secretion are described further below, and include but are not limited to Saccharomyces cerevisiae, Bacillus spp. (e.g., B. amyloliquefaciens, B. licheniformis, B. megaterium, B. stearothermophilus, and B. subtilis), or filamentous fungal organisms such as Aspergillus spp. including but not limited to A. niger, A. nidulans, A. awamori, A. oryzae, A. sojae and A. kawachi; Trichoderma reeseil; Chrysosporium lucknowense; Myceliophthora thermophilia; Fusarium venenatum; Neurospora crassa; Humicola insolens; Humicola grisea; Penicillum verruculosum; Thielavia terrestris; and teleomorphs, or anamorphs and synonyms or taxonomic equivalents thereof.

In some embodiments, a carbonic anhydrase polypeptide of the present disclosure comprises a sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to a portion of the reference sequence of SEQ ID NO:2, the portion comprising a contiguous sequence of 25, 50, 75, 100, or more than 100 contiguous amino acids of SEQ ID NO:2.

In some embodiments, the improved engineered carbonic anhydrase enzymes can comprise deletions of the naturally occurring carbonic anhydrase polypeptides as well as deletions of other improved carbonic anhydrase polypeptides. In some embodiments, each of the improved engineered carbonic anhydrase enzymes described herein can comprise deletions of the polypeptides described herein. Thus, for each and every embodiment of the carbonic anhydrase polypeptides of the disclosure, the deletions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the carbonic anhydrase polypeptides, as long as the functional activity of the carbonic anhydrase activity is maintained. In some embodiments, the deletions can comprise, 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 amino acid residues.

In some embodiments, the recombinant carbonic anhydrase polypeptides having an improved enzyme property relative to a reference polypeptide of SEQ ID NO:2, and an amino acid sequence having at least 80% identity to SEQ ID NO:2, wherein the amino acid sequence comprises one or more amino acid substitutions specifically exclude those wild-type carbonic anhydrase amino acid sequences of Methanosarcina barkeri str. Fusaro (Accession: gi|73670479|ref|YP_{—}306494.1|), Methanosarcina mazei Go1 (Accession: gi|21229190|ref|NP_{—}635112.1|), or Methanosarcina acetivorans C2A (Accession: gi|20091364|ref|NP_{—}617439.1|).

Additionally, in some embodiments, the recombinant carbonic anhydrase polypeptides having an improved enzyme property relative to a reference polypeptide of SEQ ID NO:2, and an amino acid sequence having at least 80% identity to SEQ ID NO:2, wherein the amino acid sequence comprises one or more amino acid substitutions specifically exclude sequences having one or more of the following amino acid substitutions (relative SEQ ID NO: 2) found in the wild-type carbonic anhydrase amino acid sequences of Methanosarcina barkeri str. Fusaro (Accession: gi|73670479|ref|YP_{—}306494.1|), Methanosarcina mazei Go1 (Accession: gi|21229190|ref|NP_{—}635112.1|), or Methanosarcina acetivorans C2A (Accession: gi|20091364|ref|NP_{—}617439.1|): E3G, V6E, D7S, F9V, E14A, E23V, 525T, S25V, A26E, P31S, Y34F, D36H, 540A, E44D, E44N, N50S, I59V, M65T, R72E, S73C, S73T, V75I, V80I, I87V, N88D, I94V, D96E, D96N, D965, 198L, I98Q, D102G, K104E, E105K, N112E, N113R, S120A, V122I, A126L, A127S, A127Y, D130N, A138T, F139L, S143A, K144N, V145I, N147D, R154K, R154T, A156G, I162V, M172T, A178D, K182E, K182N, P184S, V186I, A191G, S193K, V204T, H205N, E208A, K211N, E212K.

Alternatively, in some embodiments the present disclosure also contemplates recombinant carbonic anhydrase polypeptides having an improved enzyme property relative to a reference polypeptide of SEQ ID NO:2, and an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO:2, wherein the amino acid sequence comprises one or more amino acid substitutions, C-terminal fusions, or amino terminus extensions, selected from those listed in Table 2, and one or more of the following amino acid substitutions (relative SEQ ID NO: 2) found in the wild-type carbonic anhydrase amino acid sequences of Methanosarcina barkeri str. Fusaro (Accession: gi|73670479|ref|YP_{—}306494.1|), Methanosarcina mazei Go1 (Accession: gi|21229190|ref|NP_{—}635112.1|), or Methanosarcina acetivorans C2A (Accession: gi|20091364|ref|NP_{—}617439.1|): E3G, V6E, D7S, F9V, E14A, E23V, S25T, S25V, A26E, P31S, Y34F, D36H, S40A, E44D, E44N, N50S, I59V, M65T, R72E, S73C, S73T, V75I, V80I, I87V, N88D, I94V, D96E, D96N, D965, 198L, I98Q, D102G, K104E, E105K, N112E, N113R, S120A, V122I, A126L, A127S, A127Y, D130N, A138T, F139L, S143A, K144N, V145I, N147D, R154K, R154T, A156G, I162V, M172T, A178D, K182E, K182N, P184S, V186I, A191G, S193K, V204T, H205N, E208A, K211N, E212K.

In some embodiments, the present disclosure also contemplates a recombinant carbonic anhydrase polypeptides having an improved enzyme property relative to a reference polypeptide of SEQ ID NO:2, and an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the wild-type carbonic anhydrase amino acid sequences of any one of Methanosarcina barkeri str. Fusaro (Accession: gi|73670479|ref|YP_{—}306494.1|), Methanosarcina mazei Go1 (Accession: gi|21229190|ref|NP_{—}635112.1|), or Methanosarcina acetivorans C2A (Accession: gi|20091364|ref|NP_{—}617439.1|), wherein the amino acid sequence further comprises a carboxy terminal fusion of any one of the polypeptides of SEQ ID NOs: 101-118, 316-338, KAK, KA, or the single amino acid K. In some embodiments, the polypeptide further comprises one or more amino acid substitutions (relative SEQ ID NO: 2) selected from those listed in Table 2.

The polypeptides described herein are not restricted to the genetically encoded amino acids. In addition to the genetically encoded amino acids, the polypeptides described herein may be comprised, either in whole or in part, of naturally-occurring and/or synthetic non-encoded amino acids. Certain commonly encountered non-encoded amino acids of which the polypeptides described herein may be comprised include, but are not limited to: the D-enantiomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (NaI); 2-chlorophenylalanine (Ocf); 3-chlorophenylalanine (Mcf); 4 chlorophenylalanine (Pcf); 2 fluorophenylalanine (Off); 3 fluorophenylalanine (Mff); 4 fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisolencine (hIle); homoarginine (hArg); N acetyl lysine (AcLys); 2,4 diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art (see, e.g., the various amino acids provided in Fasman, 1989, CRC Practical Handbook of Biochemistry and Molecular Biology, CRC Press, Boca Raton, Fla., at pp. 3-70 and the references cited therein, all of which are incorporated by reference). These amino acids may be in either the L or D configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(δ-benzylester), Gln(xanthyl), Asn(N-δ-xanthyl), His(bom), His(benzyl), His(tos), Lys(fmoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

As described above the various modifications introduced into the naturally occurring polypeptide to generate an engineered carbonic anhydrase enzyme can be targeted to a specific property of the enzyme.

### 7.3. POLYNUCLEOTIDES ENCODING ENGINEERED CARBONIC ANHYDRASES

In another aspect, the present disclosure provides polynucleotides encoding the engineered carbonic anhydrase enzymes. The polynucleotides may be operatively linked to one or more heterologous regulatory sequences that control gene expression to create a recombinant polynucleotide capable of expressing the polypeptide. Expression constructs containing a heterologous polynucleotide encoding the engineered carbonic anhydrase can be introduced into appropriate host cells to express the corresponding carbonic anhydrase polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved carbonic anhydrase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein, including the amino acid sequences presented in Table 2.

In some embodiments, the polynucleotide comprises a nucleotide sequence encoding a recombinant carbonic anhydrase polypeptide with an amino acid sequence that has at least about 80% or more sequence identity, at least 85%, 86%, 87%, 88%, 89%, 90%, 91%, 92%, 93%, 94%, 95%, 96%, 97%, 98%, or 99% identity, or more sequence identity to any of the engineered carbonic anhydrase polypeptides described herein, i.e., a polypeptide comprising an amino acid sequence selected from the group consisting of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302. Exemplary polynucleotides encoding the engineered carbonic anhydrase are selected from SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 304, 305, 306, 307, 308, 309, 310, 311, and 312.

In some embodiments, the polynucleotides encoding the engineered carbonic anhydrases are capable of hybridizing under highly stringent conditions to a polynucleotide comprising SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 304, 305, 306, 307, 308, 309, 310, 311, and 312. These polynucleotides encode some of the recombinant carbonic anhydrase polypeptides represented by the amino acid sequences listed in Table 2.

In various embodiments, the codons are preferably selected to fit the host cell in which the recombinant carbonic anhydrase polypeptide is being produced. For example, preferred codons used in bacteria are used to express the gene in bacteria; preferred codons used in yeast are used for expression in yeast; and preferred codons used in mammals are used for expression in mammalian cells. For example, the polynucleotide of SEQ ID NO: 1 could be codon optimized for expression in E. coli, but otherwise encode the naturally occurring carbonic anhydrase of Methanosarcina thermophila.

In some embodiments, all codons need not be replaced to optimize the codon usage of the recombinant carbonic anhydrase polypeptide since the natural sequence will comprise preferred codons and because use of preferred codons may not be required for all amino acid residues. Consequently, codon optimized polynucleotides encoding the carbonic anhydrase enzymes may contain preferred codons at about 40%, 50%, 60%, 70%, 80%, or greater than 90% of codon positions of the full length coding region.

In other embodiments, the polynucleotides comprise polynucleotides that encode the recombinant carbonic anhydrase polypeptide described herein but have about 80% or more sequence identity, about 85% or more sequence identity, about 90% or more sequence identity, about 95% or more sequence identity, about 98% or more sequence identity, or 99% or more sequence identity at the nucleotide level to a reference polynucleotide encoding an engineered carbonic anhydrase.

In some embodiments, the polynucleotides encoding an engineered carbonic anhydrase comprise a nucleotide sequence comprising one or more of the following nucleotide substitutions (e.g., “silent mutations”) relative to SEQ ID NO: 119: a537g; t160a; a300g; g48t; c165t; a333t; a217t; t453g; t618g; c612t. In some embodiments, the reference polynucleotide comprising a nucleotide substitution relative to SEQ ID NO: 119 is selected from polynucleotide sequences represented by SEQ ID NO: 303, 304, 305, 306, 307, 308, 309, 310, 311, and 312.

An isolated polynucleotide encoding an improved carbonic anhydrase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2006.

For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proc. Natl. Acad. Sci. USA 75: 3727-3731), as well as the tac promoter (DeBoer et al., 11983, Proc. Natl. Acad. Sci. USA 80: 21-25). Further promoters are described in “Useful proteins from recombinant bacteria” in Scientific American, 1980, 242:74-94; and in Sambrook et al., supra.

For filamentous fungal host cells, suitable promoters for directing the transcription of the nucleic acid constructs of the present disclosure include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae those phosphate isomerase), and mutant, truncated, and hybrid promoters thereof.

In a yeast host, useful promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-488.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are described by Guo and Sherman, 1995, Mol Cell Bio 15:5983-5990.

The control sequence may also be a signal peptide coding region that codes for an amino acid sequence linked to the amino terminus of an engineered carbonic anhydrase polypeptide and directs the encoded polypeptide into the cell's secretory pathway. The 5′-end of the coding sequence of the nucleic acid sequence may inherently contain a signal peptide coding region naturally linked in translation reading frame with the segment of the coding region that encodes the secreted polypeptide. Alternatively, the 5′-end of the coding sequence may contain a signal peptide coding region that is foreign to the coding sequence. The foreign signal peptide coding region may be required where the coding sequence does not naturally contain a signal peptide coding region.

In some embodiments, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention. Accordingly, an engineered carbonic anhydrase polypeptide of the invention can be operably linked to a signal sequence derived from a bacterial species such as a signal sequence derived from a Bacillus (e.g., B. stearothermophilus, B. licheniformis, B. subtilis, and B. megaterium).

Effective signal peptide coding regions for bacterial host cells are the signal peptide coding regions obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), Bacillus megaterium enzymes (nprM, yngK, penG), and Bacillus subtilis prsA. Further signal peptides are described by Simonen and Palva, 1993, Microbiol Rev 57: 109-137.

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells can be from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a pro-enzyme or pro-polypeptide (or a zymogen in some cases). A pro-polypeptide is generally inactive and can be converted to a mature active polypeptide by catalytic or autocatalytic cleavage of the propeptide from the pro-polypeptide. The pro-peptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the carbonic anhydrase polypeptide of the present invention would be operably linked with the regulatory sequence.

Thus, in another embodiment, the present disclosure is also directed to a recombinant expression vector comprising a polynucleotide encoding an engineered carbonic anhydrase polypeptide or a variant thereof, and one or more expression regulating regions such as a promoter and a terminator, a replication origin, etc., depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector of the present invention preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol or tetracycline resistance. Suitable markers for yeast host cells are ADE2, HIS3, LEU2, LYS2, MET3, TRP1, and URA3.

Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

The expression vectors of the present invention preferably contain an element(s) that permits integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or non-homologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A on or the origins of replication of plasmids pBR322, pUC19, pACYC177 (which plasmid has the P15A ori), or pACYC184 permitting replication in E. coli, and pUB110, pE194, pTA1060, or pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes it's functioning temperature-sensitive in the host cell (see, e.g., Ehrlich, 1978, Proc Natl Acad. Sci. USA 75:1433).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present disclosure are commercially available. Suitable commercial expression vectors include p3xFLAGTMTM expression vectors from Sigma-Aldrich Chemicals, St. Louis Mo., which includes a CMV promoter and hGH polyadenylation site for expression in mammalian host cells and a pBR322 origin of replication and ampicillin resistance markers for amplification in E. coli. Other suitable expression vectors are Bacillus megaterium shuttle vector pMM1525 (Boca Scientific Inc. Boca Raton, Fla.), pBluescriptll SK(−) and pBK-CMV, which are commercially available from Stratagene, La Jolla Calif., and plasmids which are derived from pBR322 (Gibco BRL), pUC (Gibco BRL), pREP4, pCEP4 (Invitrogen) or pPoly (Lathe et al., 1987, Gene 57:193-201).

### 7.4. HOST CELLS FOR EXPRESSION OF CARBONIC ANHYDRASE POLYPEPTIDES

In another aspect, the present disclosure provides a host cell comprising a polynucleotide encoding an improved carbonic anhydrase polypeptide of the present disclosure, the polynucleotide being operatively linked to one or more control sequences for expression of the carbonic anhydrase enzyme in the host cell. Host cells for use in expressing the carbonic anhydrase polypeptides encoded by the expression vectors of the present invention are well known in the art and include but are not limited to, bacterial cells, such as E. coli, Bacillus, Lactobacillus, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells.

In some embodiments of the invention the host cell is a bacterial host cell of the Bacillus species, e.g., B. thuringiensis, B. anthracia, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. Appropriate culture mediums and growth conditions for the above-described host cells are well known in the art.

Polynucleotides for expression of the carbonic anhydrase may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

An exemplary host cell is Escherichia coli W3110. The expression vector was created by operatively linking a polynucleotide encoding an improved carbonic anhydrase into the plasmid pCK110900 (see, US application publication 20040137585) operatively linked to the lac promoter under control of the lad repressor. The expression vector also contained the P15a origin of replication and the chloramphenicol resistance gene. Cells containing the subject polynucleotide in Escherichia coli W3110 were isolated by subjecting the cells to chloramphenicol selection. Another exemplary host cell is Escherichia coli BL21.

The disclosure also provides methods for producing the recombinant carbonic anhydrase polypeptides using a host cell. In some embodiments, the method for producing a recombinant carbonic anhydrase polypeptide comprises the steps of: (a) transforming a host cell with an expression vector polynucleotide encoding the recombinant carbonic anhydrase polypeptide; (b) culturing said transformed host cell under conditions whereby said recombinant carbonic anhydrase polypeptide is produced by said host cell; and (c) recovering said recombinant carbonic anhydrase polypeptide from said host cells. In some embodiments, the methods of producing the recombinant carbonic anhydrase may be carried out wherein said expression vector comprises a secretion signal, and said cell is cultured under conditions whereby the recombinant carbonic anhydrase polypeptide is secreted from the cell. In some embodiments of the method, the expression vector comprises a polynucleotide encoding a secretion signal. In some embodiments, the secretion signal encodes a signal peptide is selected from SEQ ID NO: 313, 314, and 315.

Recovery, isolation and purification of the recombinant carbonic anhydrase polypeptide may be carried out using standard methods known by the ordinary artisan such those as described further below.

### 7.5. METHODS OF GENERATING ENGINEERED CARBONIC ANHYDRASE POLYPEPTIDES

In some embodiments, to make the improved carbonic anhydrase polynucleotides and polypeptides of the present disclosure, the naturally-occurring carbonic anhydrase enzyme that catalyzes the hydration reaction is obtained (or derived) from Methanosarcina thermolphda. In some embodiments, the parent polynucleotide sequence is codon optimized to enhance expression of the carbonic anhydrase in a specified host cell. As an illustration, the parental polynucleotide sequence encoding the wild-type carbonic anhydrase polypeptide of Methanosarcina thermophila (SEQ ID NO:1), can be assembled from oligonucleotides based upon that sequence or from oligonucleotides comprising a codon-optimized coding sequence for expression in a specified host cell, e.g., an E. coli host cell. In one embodiment, the polynucleotide can be cloned into an expression vector, placing the expression of the carbonic anhydrase gene under the control of the lac promoter and lad repressor gene. Clones expressing the active carbonic anhydrase in E. coli can be identified and the genes sequenced to confirm their identity.

The engineered carbonic anhydrase can be obtained by subjecting the polynucleotide encoding the naturally occurring carbonic anhydrase to mutagenesis and/or directed evolution methods, as discussed above. An exemplary directed evolution technique is mutagenesis and/or DNA shuffling as described in Stemmer, 1994, Proc Natl Acad Sci USA 91:10747-10751; WO 95/22625; WO 97/0078; WO 97/35966; WO 98/27230; WO 00/42651; WO 01/75767 and U.S. Pat. No. 6,537,746. Other directed evolution procedures that can be used include, among others, staggered extension process (StEP), in vitro recombination (Zhao et al., 1998, Nat. Biotechnol. 16:258-261), mutagenic PCR (Caldwell et al., 1994, PCR Methods Appl. 3:S136-S140), and cassette mutagenesis (Black et al., 1996, Proc Natl Acad Sci USA 93:3525-3529).

Methodologies for screening and identifying polypeptides for desired activities are useful in the preparation of new compounds such as modified enzymes and/or new pharmaceuticals. Directed evolution can be used to discover or enhance activity of polypeptides of commercial interest. For example, if the activity of a known catalyst is insufficient for a commercial process, directed evolution and/or other protein engineering technologies may be used to make appropriate improvements to the catalyst to improve activity on the substrate of interest. Improvements to process engineering can be developed to enhance an active enzyme and/or to optimize a microbe/enzyme for scaled-up production. Current methodologies are often limited by time and cost factors. In some instances, it may take months or years, at great expense, to find a new polypeptide with the desired activity, if one is ever found. Furthermore, the number of polypeptide variants that must be screened is often cumbersome. Thus, there is a long felt need for compositions and methods used to identify novel polypeptide variants having a desired activity.

Many methodologies directed to the design and/or identification of polypeptides having particular characteristics are known in the art. For example, methods for high-throughput screening arrays of clones in a sequential manner are presented in PCT Publication No. WO 01/32858; an in vitro selection method of screening a library of catalyst molecules is disclosed in PCT Publication No. WO 00/11211; a screening method for identifying active peptides or proteins with improved performance is disclosed in PCT Publication No. WO 02/072876 and US Patent Application Publication No. 2004/0132039; a methods for creating and screening transgenic organisms having desirable traits are disclosed in U.S. Pat. No. 7,033,781; methods for making circularly permuted proteins and peptides having novel and/or enhanced functions with respect to a native protein or peptide are disclosed in PCT Publication No. WO 2006/086607; methods for preparing variants of a catalytic polypeptide are disclosed in US Patent Application Publication No. 2003/0073109; and methods for biopolymer engineering using a variant set to model sequence-activity relationships are disclosed in PCT Publication No. WO 2005/013090; each of which is incorporated herein by reference in its entirety.

The clones obtained following mutagenesis treatment are screened for engineered carbonic anhydrase having a desired improved enzyme property. Measuring enzyme activity from the expression libraries can be performed using the standard biochemistry technique of monitoring changes in pH, either directly or indirectly, as indicated in the Examples. Similarly, and as again demonstrated in the Examples, activity of the carbonic anhydrases of the disclosure may be measured using either the forward or reverse reactions depicted in Scheme 1. Where the improved enzyme property desired is thermal stability, enzyme activity may be measured after subjecting the enzyme preparations to a defined temperature for a defined period of time and measuring the amount of enzyme activity remaining after heat treatments. Clones containing a polynucleotide encoding a carbonic anhydrase are then isolated, sequenced to identify the nucleotide sequence changes (if any), and used to express the enzyme in a host cell.

Where the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the invention can be prepared by chemical synthesis using, e.g., the classical phosphoramidite method described by Beaucage et al., 1981, Tet Left 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., Operon Technologies Inc., Alameda, Calif., and many others.

Engineered carbonic anhydrase enzymes expressed in a host cell can be recovered from the cells and or the culture medium using any one or more of the well known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic BTM from Sigma-Aldrich of St. Louis Mo.

Chromatographic techniques for isolation of the carbonic anhydrase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved carbonic anhydrase enzymes. For affinity chromatography purification, any antibody which specifically binds the carbonic anhydrase polypeptide may be used. For the production of antibodies, various host animals, including but not limited to rabbits, mice, rats, etc., may be immunized by injection with a polypeptide of the disclosure. The polypeptide may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette Guerin) and Corynebacterium parvum.

### 7.6. METHODS OF USING THE ENGINEERED CARBONIC ANHYDRASE ENZYMES

The carbonic anhydrase enzymes described herein can catalyze both the forward and reverse reactions depicted in Scheme 1 above. In certain embodiments, a carbonic anhydrase of the present disclosure can be used to hydrate carbon dioxide in the form of bicarbonate and a proton, which in turn, will be converted to carbonate and/or a mixture of bicarbonate and carbonate at an elevated pH. In other embodiments, a carbonic anhydrase of the disclosure can be used to dehydrate sequestered carbon dioxide by reaction at a relatively acidic pH.

Accordingly, in some embodiments the present disclosure provides methods for removing (e.g., extracting and sequestering) carbon dioxide from a gas stream comprising the step of contacting the gas stream with a solution comprising a recombinant carbonic anhydrase polypeptide of a recombinant carbonic anhydrase of the disclosure having an improved property (e.g., increased activity and/or thermostability), whereby carbon dioxide is removed from the gas stream by dissolving into the solution where it is converted to hydrated carbon dioxide by the carbonic anhydrase. In another embodiment, the method can comprise the further step of isolating the solution comprising the hydrated carbon dioxide and contacting the isolated solution with hydrogen ions and a recombinant carbonic anhydrase polypeptide, thereby converting the hydrated carbon dioxide to carbon dioxide gas and water. Thus, it is contemplated that the solution can be removed from contact with the gas stream (e.g., isolated after some desired level of hydrated carbon dioxide is reached) and further treated with a carbonic anhydrase to convert the bicarbonate in solution into carbon dioxide gas, which is then released from the solution and captured e.g., into a pressurized chamber.

In some embodiments, the methods for removing (e.g., extracting and sequestering) carbon dioxide from a gas stream disclosed herein can be used in processes for removing carbon dioxide from the flue gas produced by a fossil fuel (e.g., coal-fired) power plant. Equipment and processes that can employ the recombinant carbonic anhydrases in processes to remove carbon dioxide from the flue gas of fossil fuel power plants have been described—see e.g., U.S. Pat. No. 6,143,556, US patent publication no. 2007/0004023A1, and PCT publications WO98/55210A1, WO2004/056455A1, and WO2004/028667A1, each of which is hereby incorporated by reference herein.

In certain embodiments, the methods of removing carbon dioxide from a gas stream can be carried out wherein the solution is aqueous, or an aqueous co-solvent system. In some embodiments of the method, the solutions and solvent systems comprise amine compounds that exhibit improved thermodynamic and kinetic properties for the absorption of CO₂ and exhibit relatively low corrosive properties. Such solutions and solvent systems are described in e.g., WO2006/089423A1, which is hereby incorporated by reference herein. Exemplary solutions or solvent systems useful in the methods disclosed herein can comprise monoethanolamine (MEA), methyldiethanolamine (MDEA), 2-aminomethylpropanolamine (AMP), 2-(2-aminoethylamino)ethanol (AEE), triethanolamine, 2-amino-2-hydroxymethyl-1,3-propanediol (Tris), dimethyl ether of polyethylene glycol (PEG DME), piperazine, or ammonia. In some embodiments, solvent systems comprising AMP and/or MDEA are preferred due to the relatively low corrosive and degradative properties of these solvents coupled with their relatively favorable thermodynamic and kinetic properties for solvating carbon dioxide.

In some embodiments of the method, the solution is a co-solvent system comprising a ratio of water to organic solvent from about 90:10 (v/v) to about 10:90 (v/v), in some embodiments, from about 80:20 to about 20:80 (v/v), in some embodiments, from about 70:30 (v/v) to about 30:70 (v/v), and in some embodiments, from about 60:40 (v/v) to about 40:60 (v/v).

Further, the methods of removing carbon dioxide from a gas stream can be carried out wherein the recombinant carbonic anhydrase polypeptide is immobilized on a surface, for example wherein the enzyme is linked to the surface of a solid-phase particle (e.g., beads) in the solution. Methods for linking (covalently or non-covalently) enzymes to solid-phase particles (e.g., porous or non-porous beads, or solid supports) such that they retain activity for use in bioreactors are well-known in the art. Methods for treating a gas stream using immobilized enzymes are described in e.g., U.S. Pat. No. 6,143,556, US patent publication no. 2007/0004023A1, and PCT publications WO98/55210A1, WO2004/056455A1, and WO2004/028667A1, each of which is hereby incorporated by reference herein.

As noted above, any of the carbonic anhydrase polypeptides described herein, including those exemplified in Table 2, can be used in the methods. Moreover, in some embodiments, the methods can use a carbonic anhydrase polypeptides comprising an amino acid sequence that is at least about 85%, 86%, 87%, 88%, 89%, 90%, 91%, 92%, 93%, 94%, 95%, 96%, 97%, 98%, or 99% identical to the amino acid sequence of the Methanosarcina thermophila carbonic anhydrase of SEQ ID NO:2, and, further, that comprises, as compared to the amino acid sequence of the Methanosarcina thermophila carbonic anhydrase of SEQ ID NO:2, at least one amino acid substitution selected from the group consisting of: residue at position 2 is an aliphatic or non-polar amino acid selected from the group consisting of alanine, leucine, isoleucine, valine, glycine, and methionine, or a polar amino acid selected from the group consisting of asparagine, serine, and threonine, or a constrained amino acid selected from the group consisting of proline and histidine; residue at position 3 is an aliphatic or non-polar amino acid selected from the group consisting of alanine, leucine, isoleucine, valine, glycine, and methionine, or an aromatic amino acid selected from phenylalanine, tyrosine, or tryptophan; residue at position 6 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 7 is a polar amino acid selected from the group consisting of asparagine, glutamine, serine, and threonine, or a constrained amino acid selected from the group consisting of proline and histidine; residue at position 8 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 10 is an aliphatic or non-polar amino acid selected from the group consisting of alanine, leucine, isoleucine, valine, glycine, and methionine, or an aromatic amino acid selected from phenylalanine, tyrosine, or tryptophan; residue at position 11 is a constrained amino acid selected from the group consisting of proline and histidine; residue at position 14 is an aromatic amino acid selected from phenylalanine, tyrosine, or tryptophan; residue at position 16 is an aliphatic or non-polar amino acid selected from the group consisting of alanine, leucine, isoleucine, valine, glycine, and methionine; residue at position 22 is an aliphatic or non-polar amino acid selected from the group consisting of alanine, leucine, isoleucine, valine, glycine, and methionine, or a basic amino acid selected from the group consisting of lysine and arginine; residue at position 23 is a basic amino selected from the group consisting of lysine and arginine, or a non-polar amino acid selected from the group consisting of alanine, leucine, isoleucine, valine, glycine, and methionine, or a polar amino acid selected from the group consisting of asparagine, glutamine, serine, and threonine; residue at position 26 is a polar amino acid selected from the group consisting of asparagine, glutamine, serine, and threonine; residue at position 27 is a non-polar amino acid selected from the group consisting of alanine, leucine, isoleucine, valine, glycine, and methionine, or an acidic amino acid selected from aspartic acid and glutamic acid; residue at position 31 is a cysteine, or an acidic amino acid selected from aspartic acid and glutamic acid, or a polar amino acid selected from the group consisting of asparagine, glutamine, serine, and threonine; residue at position 33 is an aliphatic or non-polar amino acid selected from the group consisting of alanine, leucine, isoleucine, valine, glycine, and methionine; residue at position 36 is an aliphatic or non-polar amino acid selected from the group consisting of alanine, leucine, isoleucine, valine, glycine, and methionine, or a constrained amino acid selected from the group consisting of proline and histidine; residue at position 37 is a constrained amino acid selected from the group consisting of proline and histidine; residue at position 40 is an aliphatic or non-polar amino acid selected from the group consisting of alanine, leucine, isoleucine, valine, glycine, and methionine, or a cysteine; residue at position 44 is an aliphatic or non-polar amino acid selected from the group consisting of alanine, leucine, isoleucine, valine, glycine, and methionine, or a polar amino acid selected from the group consisting of asparagine, glutamine, serine, and threonine, or a constrained amino acid selected from the group consisting of proline and histidine; residue at position 46 is an aliphatic or non-polar amino acid selected from the group consisting of alanine, leucine, isoleucine, valine, glycine, and methionine, or a polar amino acid selected from the group consisting of asparagine, glutamine, and serine, or an acidic amino acid selected from aspartic acid and glutamic acid; residue at position 56 is cysteine or a constrained amino acid selected from the group consisting of proline and histidine; residue at position 57 is an aliphatic or non-polar amino acid selected from the group consisting of alanine, leucine, isoleucine, valine, glycine, and methionine; residue at position 58 is an aliphatic or non-polar amino acid selected from the group consisting of alanine, leucine, isoleucine, valine, glycine, and methionine; residue at position 87 is a polar amino acid selected from the group consisting of asparagine, glutamine, serine, and threonine; residue at position 90 is a basic amino acid selected from the group consisting of lysine and arginine; residue at position 95 is a polar amino acid selected from the group consisting of asparagine, glutamine, serine, and threonine, or a basic amino acid selected from the group consisting of lysine and arginine; residue at position 98 is an aliphatic or non-polar amino acid selected from the group consisting of alanine, leucine, valine, glycine, and methionine, or a basic amino acid selected from the group consisting of lysine and arginine; residue at position 104 is a polar amino acid selected from the group consisting of asparagine, glutamine, serine, and threonine; residue at position 105 is a polar amino acid selected from the group consisting of asparagine, glutamine, serine, and threonine, or an aromatic amino acid selected from phenylalanine, tyrosine, or tryptophan; residue at position 122 is an aliphatic or non-polar amino acid selected from the group consisting of alanine, leucine, isoleucine, glycine, and methionine; residue at position 127 is an acidic amino acid selected from aspartic acid and glutamic acid, or a basic amino acid selected from the group consisting of lysine and arginine, or an aromatic amino acid selected from phenylalanine, tyrosine, or tryptophan; residue at position 131 is a polar amino acid selected from the group consisting of asparagine, glutamine, serine, and threonine; residue at position 136 is a polar amino acid selected from the group consisting of asparagine, glutamine, serine, and threonine; residue at position 137 is an aliphatic or non-polar amino acid selected from the group consisting of alanine, leucine, isoleucine, valine, glycine, and methionine; residue at position 138 is a polar amino acid selected from the group consisting of asparagine, glutamine, serine, and threonine; residue at position 139 is an aliphatic or non-polar amino acid selected from the group consisting of alanine, leucine, isoleucine, valine, glycine, and methionine; residue at position 142 is a polar amino acid selected from the group consisting of asparagine, glutamine, serine, and threonine; residue at position 147 is a polar amino acid selected from the group consisting of asparagine, glutamine, serine, and threonine, or a constrained amino acid selected from the group consisting of proline and histidine; residue at position 149 is a polar amino acid selected from the group consisting of asparagine, glutamine, serine, and threonine; residue at position 156 is a polar amino acid selected from the group consisting of asparagine, glutamine, serine, and threonine; residue at position 161 is a polar amino acid selected from the group consisting of asparagine, glutamine, or serine; residue at position 165 is a polar amino acid selected from the group consisting of asparagine, glutamine, serine, and threonine, or a basic amino acid selected from the group consisting of lysine and arginine; residue at position 191 is a constrained amino acid selected from the group consisting of proline and histidine; residue at position 194 is an aliphatic or non-polar amino acid selected from the group consisting of alanine, leucine, isoleucine, valine, glycine, and methionine, or an acidic amino acid selected from aspartic acid and glutamic acid; residue at position 195 is a non-polar amino acid selected from the group consisting of alanine, leucine, isoleucine, valine, glycine, and methionine; residue at position 203 is an aliphatic or non-polar amino acid selected from the group consisting of alanine, leucine, isoleucine, valine, glycine, and methionine; residue at position 204 is an aliphatic or non-polar amino acid selected from the group consisting of alanine, leucine, isoleucine, valine, glycine, and methionine, or a polar amino acid selected from the group consisting of asparagine, glutamine, serine, and threonine; residue at position 208 is an aliphatic or non-polar amino acid selected from the group consisting of alanine, leucine, isoleucine, valine, glycine, and methionine; residue at position 212 is a basic amino acid selected from the group consisting of arginine and lysine, or a non-polar amino acid selected from the group consisting of alanine, leucine, isoleucine, valine, glycine, and methionine; residue at position 213 is an aliphatic or non-polar amino acid selected from the group consisting of alanine, leucine, isoleucine, valine, glycine, and methionine; and residue at position 214 is a cysteine, or an acidic amino acid selected from aspartic acid and glutamic acid, or an aliphatic or non-polar amino acid selected from the group consisting of alanine, leucine, isoleucine, valine, glycine, and methionine, or a basic amino acid selected from the group consisting of lysine and arginine, or an aromatic amino acid selected from phenylalanine, tyrosine, or tryptophan, or a constrained amino acid selected from the group consisting of proline and histidine. The forgoing improved carbonic anhydrase polypeptides may further comprise additional modifications, including substitutions, deletions, insertions, or combinations thereof. The substitutions can be non-conservative substitutions, conservative substitutions, or a combination of non-conservative and conservative substitutions. In some embodiments, these carbonic anhydrase polypeptides can have optionally from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 mutations at other amino acid residues. In some embodiments, the number of modifications can be 1, 2, 3, 4, 5, 6, 7, 8, 9, 10, 11, 12, 14, 15, 16, 18, 20, 22, 24, 26, 30, 35 or about 40 other amino acid residues.

In some embodiments, the methods can use an improved carbonic anhydrase polypeptide of the present disclosure that comprises an amino acid sequence that is at least about 85%, 86%, 87%, 88%, 89%, 90%, 91%, 92%, 93%, 94%, 95%, 96%, 97%, 98%, or 99% identical to the amino acid sequence of the Methanosarcina thermophila carbonic anhydrase of SEQ ID NO:2, and that further comprises, as compared to the amino acid sequence of the Methanosarcina thermophila carbonic anhydrase of SEQ ID NO:2, at least one amino acid substitution selected from the group consisting of: residue at position 2 is alanine, histidine, asparagine, or proline; residue at position 3 is alanine, leucine, or tryptophan; residue at position 6 is methionine, or glutamine; residue at position 7 is proline, or serine; residue at position 8 is alanine, or glutamine; residue at position 10 is valine, or tryptophan; residue at position 11 is proline; residue at position 14 is phenylalanine; residue at position 16 is valine; residue at position 22 is isoleucine, or lysine; residue at position 23 is glycine, lysine, or serine; residue at position 26 is serine; residue at position 27 is glutamic acid, or leucine; residue at position 31 is cysteine, aspartic acid, or glutamine; residue at position 33 is glycine; residue at position 36 is alanine, or histidine; residue at position 37 is histidine; residue at position 40 is cysteine, or valine; residue at position 44 is alanine, proline, or glutamine; residue at position 46 is aspartic acid, leucine, serine, or valine; residue at position 56 is cysteine, or histidine; residue at position 57 is valine; residue at position 58 is valine; residue at position 87 is threonine; residue at position 90 is lysine; residue at position 95 is glutamine; residue at position 98 is lysine, or valine; residue at position 104 is glutamine; residue at position 105 is threonine, or tryptophan; residue at position 122 is isoleucine; residue at position 127 is glutamic acid, arginine, or tryptophan; residue at position 131 is asparagine; residue at position 136 is glutamine; residue at position 137 is glycine; residue at position 138 is serine; residue at position 139 is methionine, or valine; residue at position 142 is glutamine; residue at position 147 is alanine, or histidine; residue at position 149 is serine; residue at position 156 is threonine; residue at position 161 is asparagine; residue at position 165 is asparagine, or lysine; residue at position 191 is proline; residue at position 194 is alanine, glutamic acid, or glycine; residue at position 195 is methionine; residue at position 203 is isoleucine; residue at position 204 is glycine, glutamine, or threonine; residue at position 208 is valine; residue at position 212 is arginine, glycine, or lysine; residue at position 213 is leucine; and residue at position 214 is cysteine, aspartic acid, glutamic acid, histidine, lysine, methionine, or tryptophan.

In certain embodiments, the methods can be carried out using a recombinant carbonic anhydrase polypeptide of the present disclosure, wherein the polypeptide comprises an amino acid sequence selected from the group consisting of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302. In some embodiments, the foregoing improved recombinant carbonic anhydrase polypeptides useful with the methods disclosed herein may further comprise additional modifications, including substitutions, deletions, insertions, or combinations thereof. The substitutions can be non-conservative substitutions, conservative substitutions, or a combination of non-conservative and conservative substitutions. In some embodiments, these carbonic anhydrase polypeptides can have optionally from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 mutations at other amino acid residues. In some embodiments, the number of modifications can be 1, 2, 3, 4, 5, 6, 7, 8, 9, 10, 11, 12, 14, 15, 16, 18, 20, 22, 24, 26, 30, 35 or about 40 other amino acid residues.

In some embodiments, the methods of the present disclosure use a carbonic anhydrase comprising the amino acid sequence selected from the group consisting of SEQ ID NO:4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In some embodiments, the methods of the present disclosure use a carbonic anhydrase comprising the amino acid sequence selected from the group consisting of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 60, 62, 64, 66, 68, 70, 72, 74, 76, 78, 80, 82, 84, 86, 88, 90, 92, 94, 96, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In other embodiments, the methods of the present disclosure use a carbonic anhydrase comprising the amino acid sequence selected from the group consisting of SEQ ID NO: 4, 6, 10, 12, 14, 16, 20, 22, 24, 28, 36, 38, 44, 50, 56, 60, 62, 64, 66, 68, 70, 72, 74, 76, 78, 80, 82, 84, 86, 88, 90, 94, 96, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In particular embodiments, the methods of the present disclosure use a carbonic anhydrase comprising the amino acid sequence selected from the group consisting of SEQ ID NO: 4, 6, 10, 16, 20, 22, 24, 60, 62, 64, 66, 68, 70, 72, 74, 76, 78, 80, 82, 84, 86, 88, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In particular embodiments, the methods of the present disclosure use a carbonic anhydrase comprising the amino acid sequence selected from the group consisting of SEQ ID NO: 4, 6, 16, 22, 24, 60, 62, 64, 66, 68, 70, 72, 74, 76, 78, 80, 84, 86, 88, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In particular embodiments, the methods of the present disclosure use a carbonic anhydrase comprising the amino acid sequence selected from the group consisting of SEQ ID NO: 4, 22, 24, 60, 62, 64, 66, 68, 70, 72, 74, 76, 78, 80, 84, 86, 88, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

In various embodiments, the methods of using the recombinant carbonic anhydrase polypeptides disclosed herein may be carried out under a range of different reaction conditions. The ordinary artisan will recognize that certain reaction conditions can favor the hydration of carbon dioxide to bicarbonate. The recombinant carbonic anhydrase polypeptides disclosed herein are biocatalysts with the improved abilities (e.g., thermal stability, solvent stability, and/or base stability) to catalyze hydration of carbon dioxide to bicarbonate under a range of such reaction conditions.

Accordingly, in some embodiments, the methods of using recombinant carbonic anhydrase polypeptides disclosed herein can be carried out in the presence of from about 0.1 M K2CO₃ to about 5 M K2CO₃, from about 0.2 M K2CO₃ to about 4 M K2CO₃, or from about 0.3 M K2CO₃ to about 3 M K2CO₃.

In some embodiments, the methods of using recombinant carbonic anhydrase polypeptides disclosed herein can be carried out at increased temperature ranges of from about 50° C. to 100° C., from about 60° C. to 90°, or from about 70° C. to 80°, and wherein said polypeptide is exposed to the increased temperature for a period of time from about 5 minutes to about 180 minutes, from about 10 minutes to about 120 minutes, or from about 15 minutes to about 60 minutes.

In some embodiments, the methods of using recombinant carbonic anhydrase polypeptides disclosed herein can be carried out under a combination of challenging conditions, including, e.g., in the presence of from about 0.1 M K2CO₃ to about 0.5 M K2CO₃ after heating the recombinant carbonic anhydrase polypeptide and the reference polypeptide at a temperature within the range of from about 50° C. to 100° C. for a period of time within the range of from about 5 minutes to about 180 minutes.

In some embodiments, the methods of using recombinant carbonic anhydrase polypeptides disclosed herein can be carried out in the presence of a range of solvent conditions, including e.g., in an aqueous solution (e.g., a buffered solution), a non-aqueous solvent solution (e.g., an organic solvent), or a co-solvent solution (e.g., an aqueous-organic co-solvent system). In some embodiments, the solution, or co-solvent system used in the methods, comprises a solvent that thermodynamically and/or kinetically favors the solvation of CO₂ from a gas-solvent interface.

In particular embodiments, the carbonic anhydrase-catalyzed hydration reactions described herein are carried out in a solvent. Suitable solvents include water (e.g., aqueous solution), and mixtures of water and an organic reagent or solvent (e.g., monoethanolamine, methyldiethanolamine, and 2-aminomethylpropanolamine, dimethyl ether of polyethylene glycol, piperazine, ammonia, and the like) or aqueous carbonate mixtures. In certain embodiments, aqueous solvents, including water and aqueous co-solvent systems, are used.

Exemplary aqueous co-solvent systems have water and one or more organic solvents. In general, an organic solvent component of an aqueous co-solvent system is selected such that it does not completely inactivate the carbonic anhydrase enzyme. Appropriate co-solvent systems can be readily identified by measuring the enzymatic activity of the specified engineered carbonic anhydrase enzyme in the candidate solvent system, utilizing an enzyme activity assay, such as those described herein.

In some embodiments, the methods of using recombinant carbonic anhydrase polypeptides disclosed herein can be carried out in the presence of a co-solvent selected from the group consisting of: monoethanolamine (MEA), methyldiethanolamine (MDEA), 2-aminomethylpropanolamine (AMP), 2-(2-aminoethylamino)ethanol (AEE), triethanolamine, 2-amino-2-hydroxymethyl-1,3-propanediol (Tris), dimethyl ether of polyethylene glycol (PEG DME), piperazine, ammonia, and mixtures thereof. In some embodiments, the methods can be carried out in the presence of from about 0.5 M AMP to about 3.0 M AMP, from about 1.0 M AMP to about 2.0 M AMP, or from about 1.25 M AMP to about 1.75 M AMP.

The organic solvent component of an aqueous co-solvent system may be miscible with the aqueous component, providing a single liquid phase, or may be partly miscible or immiscible with the aqueous component, providing two liquid phases. In general, the ratio of water to organic solvent in the co-solvent system is in the range of from about 90:10 (v/v) to about 10:90 (v/v), and typically from about 80:20 (v/v) to about 20:80 (v/v), from about 70:30 (v/v) to about 30:70 (v/v), or from about 60:40 (v/v) to about 40:60 (v/v). The co-solvent system may be pre-formed prior to addition to the reaction mixture, or it may be formed in situ in the reaction vessel.

The aqueous solvent (water or aqueous co-solvent system) may be pH buffered or unbuffered. Generally, hydration of carbon dioxide can be carried out at a pH of about pH 9 or above or at a pH of about pH 10 or above, usually in the range of from about 8 to about 12.

In some embodiments, the methods can be carried out in a solution at a basic pH that thermodynamically and/or kinetically favors the solvation of CO₂—e.g., from about pH 8 to about pH 12. Accordingly, in some embodiments, the rate is determined at a pH of from about pH 8 to about pH 12, from about pH 9 to about pH 11.5, or from about pH 9.5 to pH 11.

In other embodiments, release (dehydration) of captured carbon dioxide (e.g., as bicarbonate) is carried out at a pH of about 9 or below, usually in the range of from about pH 5 to about pH 9. In some embodiments, the dehydration is carried out at a pH of about 8 or below, often in the range of from about pH 6 to about pH 8.

During the course of both the hydration and the dehydration reactions, the pH of the reaction mixture may change. The pH of the reaction mixture may be maintained at a desired pH or within a desired pH range by the addition of an acid or a base during the course of the reaction. Alternatively, the pH may be controlled by using an aqueous solvent that comprises a buffer. Suitable buffers to maintain desired pH ranges are known in the art and include, for example, carbonate, HEPES, triethanolamine buffer, and the like. The ordinary artisan will recognize that other combinations of buffering and acid or base additions known in the art may also be used.

In carrying out the reactions depicted in Scheme 1, the engineered carbonic anhydrase enzyme may be added to the reaction mixture in the form of the purified enzymes, whole cells transformed with a gene encoding the enzyme, and/or cell extracts and/or lysates of such cells.

Whole cells transformed with a gene encoding the engineered carbonic anhydrase enzyme or cell extracts and/or lysates thereof, may be employed in a variety of different forms, including solid (e.g., lyophilized, spray-dried, and the like) or semisolid (e.g., a crude paste) forms.

The cell extracts or cell lysates may be partially purified by precipitation (ammonium sulfate, polyethyleneimine, heat treatment or the like, followed by a desalting procedure prior to lyophilization (e.g., ultrafiltration, dialysis, and the like). Any of the cell preparations may be stabilized by crosslinking using known crosslinking agents, such as, for example, glutaraldehyde or immobilization to a solid phase (e.g., Eupergit C, and the like) or by the crosslinking of protein crystals or precipitated protein aggregate particles.

Suitable conditions for carrying out the carbonic anhydrase-catalyzed hydration reactions described herein include a wide variety of conditions which can be optimized by routine experimentation that includes, but is not limited to, contacting the engineered carbonic anhydrase enzyme and substrate at an experimental pH and temperature and detecting product, for example, using the methods described in the Examples provided herein.

The carbonic anhydrase catalyzed hydration (absorption) is typically carried out at a temperature in the range of from about 25° C. to about 85° C. or higher. In some embodiments, the reaction is carried out at a temperature in the range of from about 40° C. to about 80° C. In still other embodiments, it is carried out at a temperature in the range of from about 50° C. to about 75° C.

The carbonic anhydrase catalyzed dehydration (stripping) is typically carried out at a temperature in the range of from about 25° C. to about 85° C. or higher, optionally at reduced pressure.

### EXAMPLES

Various features and embodiments of the disclosure are illustrated in the following representative examples, which are intended to be illustrative, and not limiting.

### Example 1

### Construction of a Gene Encoding the Wild Type Carbonic Anhydrase Enzymes of Methanosarcina thermophila and Construction of Expression Vectors

The gene coding for the carbonic anhydrase, CAM, from Methanosarcina thermophila TM-1 was synthesized based upon the known sequence disclosed as GenBank Accession No. U08885. The gene was synthesized by GenScript (Piscataway, N.J.), cloned into the SfiI cloning sites of expression vector, pCK110900, under the control of a lac promoter and lacIq repressor gene, creating plasmid pCK900-cam. The expression vector also contained the P15a origin of replication and the chloramphenicol resistance gene. The plasmid was transformed into an E. coli expression host, E coli BL21, using standard methods. Several clones were sequenced to confirm the correct DNA sequence. A sequence designated CAM001 (SEQ ID NO: 1) was used as the starting material for all further experiments.

Polynucleotides encoding carbonic anhydrases of the present invention were similarly cloned into vector pCK110900, then transformed and expressed from E. coli BL21, using standard methods.

### Example 2

### Carbonic Anhydrase Enzyme Preparation

Shake Flask Preparation: A single microbial colony of E. coli containing a plasmid carrying the carbonic anhydrase gene of interest was inoculated into 50 ml Luria Bertani broth containing 30 μg/ml chloramphenicol and 1% glucose. Cells were grown overnight (at least 16 hrs) in an incubator at 30° C. with shaking at 250 rpm. The culture was diluted into 250 ml 2YT (16 g/L bacto-tryptone, 10 g/L yeast extract, 5 g/L sodium chloride 30 μg/ml chloramphenicol) in 1 liter flask to an optical density at 600 nm (OD600) of 0.1 and allowed to grow at 30° C. Expression of carbonic anhydrase gene was induced with 1 mM IPTG, and ZnSO₄ added to a final concentration of 0.5 mM when the OD600 of the culture was 0.6 to 0.8 and then the broth was incubated overnight (at least 16 hrs). Cells were harvested by centrifugation (5000 rpm, 15 min, 4° C.) and the supernatant discarded. The cell pellet was resuspended with 3 ml of lysis buffer per gram of cell wet weight and allowed to incubate at room temperature. The lysis buffer consisted of 25 mM HEPES, 0.5 mg/mL lysozyme and 0.25 mg/mL PMBS, pH 8.2. The resuspended cells were then passed (two passes) through a Constant Systems Cell Disruptor System (Constant Systems, UK), at a pressure of 33.6 kpsi. Soluble and insoluble cell contents were separated by centrifugation at 12,000 rpm for 20 minutes at 4° C. The clarified lysate was then lyophilized and stored at −20 degrees C.

High Throughput Expression and Production of Carbonic Anhydrase: On day 1, freshly transformed colonies on a Q-tray (Genetix USA, Inc. Beaverton, Oreg.) containing 200 ml LB agar+1% glucose, 30 mg/ml chloramphenicol were picked using a Q-bot® robot colony picker (Genetix USA, Inc., Beaverton, Oreg.) into shallow 96 well plates containing media (70 μL/well Luria Broth (LB)+1% glucose, 30 μg/ml chloramphenicol) for overnight growth at 30° C., 225 revolutions per minute (rpm), 85% relative humidity (RH). A negative control (E. coli BL21 with empty vector) and a positive control (E. coli BL21 with vector containing CAM001, SEQ ID NO: 1) were included. These master well plate cultures were covered with AirPore™ microporous tape (Qiagen, Inc., Valencia, Calif.). These overnight cultures were diluted 40-fold into fresh 2YT (24 g/L yeast extract, 12 g/L bacto-tryptone containing 30 μg/mlchloramphenicol) in deep 96 well plates and after 2.5 hours of growth at 250 rpm shaker 30° C. (OD should equal 0.7-0.8), 1/10 volume 10 mM IPTG (isopropyl thiogalactoside) and 5 mM ZnSO₄ were added (1 mM final IPTG and 0.5 mM final ZnSO₄). The cultures were allowed to grow another 5 hours at 30° C. Cells were pelleted via centrifugation and lysed in 0.20 ml lysis buffer by shaking at room temperature for 1 hour. Lysis buffer contained 25 mM Hepes buffer (pH 8.3), 0.5 mg/ml PMBS (polymixin B sulfate), 0.2 mg/ml lysozyme, 1 mM DTT (dithiothreitol). The plate was centrifuged at 4000 rpm, 4° C., for 25 minutes and the clarified lysate assayed for carbonic anhydrase activity using the assays described below.

### Example 3

### Purification of Carbonic Anhydrase

Clarified cell lysate was applied to a DEAE FF column (GE Biosciences HiPrep 16/10 DEAE FF) that was equilibrated in 75% Buffer A (20 mM HEPES, pH 8.0), 25% Buffer B (20 mM HEPES, 1 M NaCl, pH 8.0) on an AKTA FPLC (GE Healthcare Bio-Sciences Corp., NJ). After injection of the cleared lysate, a gradient from 25% Buffer B to 55% Buffer B was run over 20 column volumes at a flow rate of 4.5 mL/min. The maximum wild type carbonic anhydrase enzyme peak eluted at 346 mM NaCl, or 34.6% Buffer B. Other CA enzyme variants were purified using this method and eluted under similar conditions.

### Example 4

### Carbonic Anhydrase Activity Assay (CO₂ Dehydration)

The assay was adapted from the Wilbur-Anderson assay (Wilbur & Anderson, Journal of Biological Chemistry (1948) 176:147-154). The clarified lysate from Example 2 was assayed for carbonic anhydrase activity in an assay solution containing 150 mM K2CO₃²⁻, pH 10.9, 400 μM phenolphthalein. The activity assay was carried out in Whatman 96-well plates with 300 μL volume wells (GE Healthcare, Inc. Piscataway, N.J.). Briefly, the assay was carried out as follows. The assay reaction mix was prepared by adding 20 μL of clarified lysate to 180 μL assay solution in a plate well. The assay reaction mix then was allowed to equilibrate in a 20% CO₂ atmosphere for 25 minutes at room temperature. During this equilibration period, the CO₂ hydration reaction commenced causing the pH indicator dye, phenolphthalein, to turn colorless due to the accumulation of protons. Once equilibrated, the now clear assay reaction mix was removed from the 20% CO₂ atmosphere, and the HCO₃⁻ dehydration reaction rate was determined by monitoring the change in absorbance at 550 nm over time using a SpectraMax M2 plate reader (MDS Analytical Technologies, Inc., Sunnyvale, Calif.). The onset time (in seconds) at which the absorbance at 550 nm of the assay reaction mix reached a set optical density value (typically OD₅₅₀=0.15) was recorded. Carbonic anhydrase activity was calculated from the onset time using the equation:

2(t₀−t)/t

where t is the onset time for the assay reaction mix (i.e., the sample) and t₀ is the onset time in seconds for a negative reaction (i.e., control reaction) to reach the set OD₅₅₀ value. In these experiments, the negative reaction contained “negative lysates” from E coli BL21 cells transformed with pCK110900 vector alone. The negative lysates typically exhibited some carbonic anhydrase activity due to the presence of some residual E. coli “background” carbonic anhydrase activity.

### Example 5

### Assay of Heat Treated Carbonic Anhydrase Enzymes

Carbonic anhydrase enzyme to be tested (clarified lysate or lyophilized powder dissolved at a concentration of 30 mM in 25 mM Hepes buffer (pH 8.3)) were incubated at 75° C. for 30 minutes or 1 hour. The heat treated carbonic anhydrase enzyme (20 μL) was added to 180 μL of a solution containing 150 mM K2CO₃ (pH 10.9), 400 μM phenolphthalein and assayed using the dehydration assay described above in Example 4.

As indicated in Table 3, heat treated carbonic anhydrase enzyme variants were identified that have improved enzymatic activity over the heat treated wild type enzyme of SEQ ID NO: 2.

TABLE 3

| | | | |
|---|---|---|---|
| SEQ ID | | Fold | Fold |
| NO: | Amino Acid Substitutions | Improvement | Improvement |
| (nt/aa) | (As Compared To SEQ ID NO: 2) | over WT^{a} | over WT^{b} |

| | | | |
|---|---|---|---|
| 3/4 | E212K T213L S214H and 21 additional amino | 8.3 | |
| | acids (KAKLATITITIREEQMGKLDL) attached | | |
| | at the carboxy terminus | | |
| 5/6 | S40V S58V E90K | 5.3 | |
| 7/8 | S40V M56C S58V | 2.7 | |
| 9/10 | M56H | 4.9 | |
| 11/12 | S40V S58V | 3.5 | |
| 13/14 | M56H S58V | 3.0 | |
| 15/16 | M56H | 5.4 | |
| 17/18 | S40V M56C S58V | 2.5 | |
| 19/20 | M56H I87T | 3.8 | |
| 21/22 | M56H E212G | 6.7 | |
| 23/24 | D7S E212K T213L S214H and 21 additional | 7.5 | 10 |
| | amino acids (KAKLATITITIREEQMGKLDL) | | |
| | attached at the carboxy terminus | | |
| 25/26 | D7S T195M | 2.1 | |
| 27/28 | D7S E23K G165N | 3.3 | |
| 29/30 | D7S | ND | 5 |
| 31/32 | D7S E95K D131N T195M | 2.9 | |
| 33/34 | D7S T195M | 2.1 | |
| 35/36 | D7S E95K T195M | 3.5 | 6.5 |
| 37/38 | D7S T195M | 2.3 | |
| 39/40 | D7S D131N G165N T195M | 2.6 | |
| 41/42 | D7S E95Q G165N T195M | 2.4 | |
| 43/44 | D7S E95K D131N G165N T195M | 3.6 | |
| 45/46 | D7S E95Q D131N G165N T195M | 2.6 | |
| 47/48 | D7S D131N T195M | 2.4 | |
| 49/50 | D7S D131N G165N E208V | ND | 6.5 |
| 51/52 | D7S E95Q T195M | 2.6 | |
| 53/54 | D7S D131N T195M | 2.5 | |
| 55/56 | D7S E95K D131N G165N T195M | 3. | 6.5 |

^{a}activity measured after heating at 75° C. for 30 min
^{b}activity measured after heating at 75° C. for 1 hour
Data are expressed as the fold-improvement over the rate observed with the wild-type (WT) carbonic anhydrase of SEQ ID NO: 2.
ND: not determined

### Example 6

### Further Characterization of Improved Carbonic Anhydrase Enzymes

Purified carbonic anhydrase enzyme variants with improved characteristics were challenged at higher temperature in carbonate buffer The CA enzymes to be tested were dissolved at a concentration of 30 mM in 150 mM K2CO₃ buffer (pH 10.9) and incubated at the indicated temperature for a predetermined period of time. The heat challenged enzymes were assayed using the dehydration assay described above. (N=3)

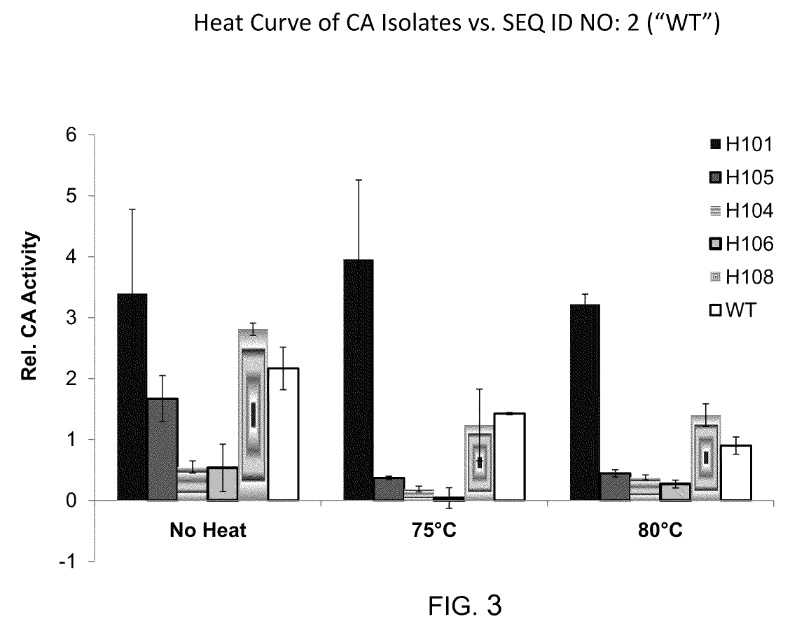

As indicated in FIG. 3, with heating to 75° C. for 30 minutes in 150 mM K2CO₃ buffer (pH 10.9), the recombinant carbonic anhydrase of SEQ ID NO: 24 (H101) was at least twice as active as the wild-type enzyme of SEQ ID NO: 2 (WT). Even after heating at 80° C. for 30 minutes, recombinant carbonic anhydrase of SEQ ID NO:24 (H101) was 2.5 to 3-fold more active than the wild type enzyme of SEQ ID NO:2 (WT). The recombinant carbonic anhydrase of SEQ ID NO: 4 (H108) was more sensitive to heat treatment in carbonate buffer than SEQ ID NO: 24, exhibiting a decrease in stability at 30 minutes in 75° or 80° C.

The other best variant hits from the high throughput screening assay, SEQ ID NO: 50 (H105), SEQ ID NO: 36 (H104), and SEQ ID NO: 56 (H106) did not show improved stability at 75° C. and 80° C. when compared with WT at equal protein concentration. These variants likely showed improvement during HTP assay screen due to increased protein expression, i.e., they were produced in greater quantity during induction and growth. Thus, the variant polypeptides of SEQ ID NOs: 36, 50, and 56 exhibit the improved property of increased expression.

### Example 7

### Carbonic Anhydrase Activity: Solvent Tolerance

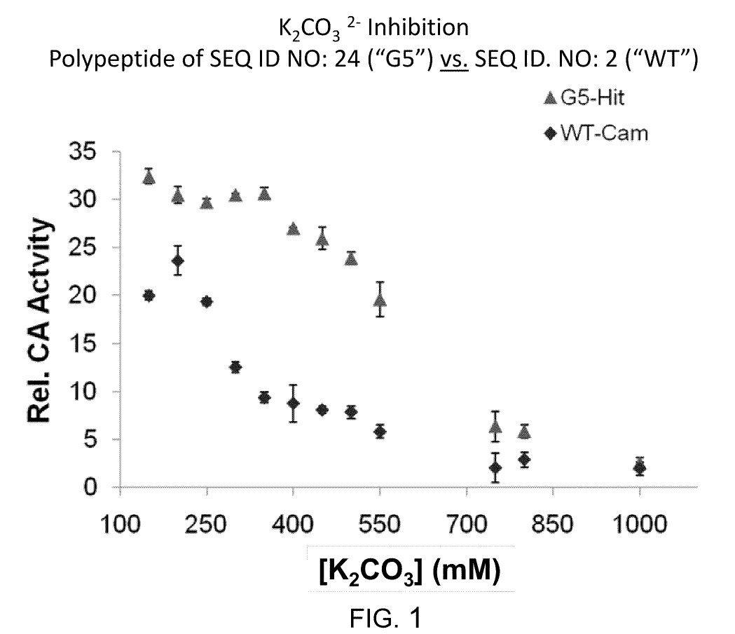

The enzymatic activity of the recombinant carbonic anhydrase of SEQ ID NO:24 (in crude form—i.e. as a bacterial cell lysate), as well as that of the wild type enzyme of SEQ ID NO:2 were determined in the presence of increasing concentrations of K2CO₃. The enzymes were assayed using the dehydration assay described above with the modification that the K2CO₃ concentration used covered a range of 0.15 M to 1 M. The data obtained are presented in FIG. 1, which indicates that the carbonic anhydrase of SEQ ID NO:24 was more active than the wild type control in the presence of increased levels of K2CO₃.

### Example 8

### Carbonic Anhydrase Activity with Heat-Treated Enzymes: Solvent Tolerance

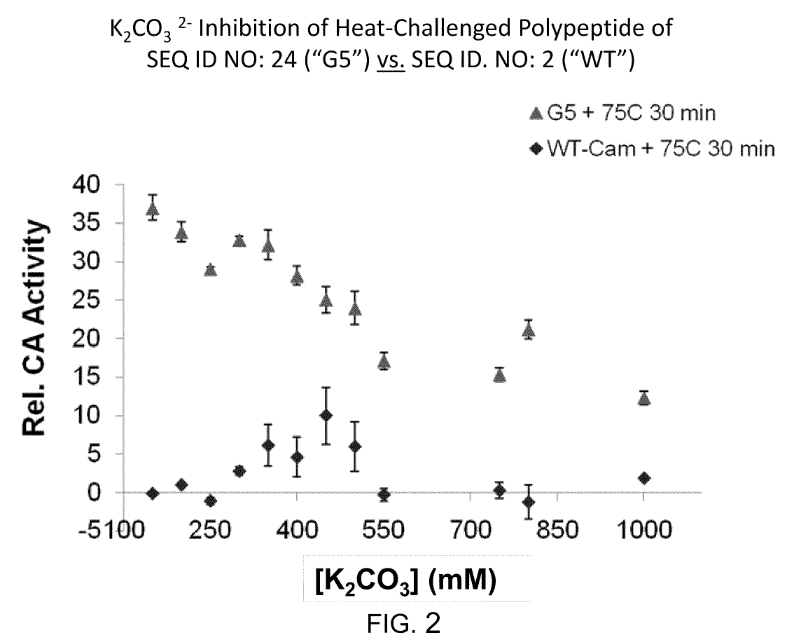

The enzymatic activities of pre-heated (75° C., 30 minutes) recombinant carbonic anhydrase of SEQ ID NO:24 (in crude form—i.e. as a bacterial cell lysate), as well as that of the similarly treated wild type enzyme of SEQ ID NO:2 were determined in the presence of increasing concentrations of K2CO₃. The enzymes were assayed using the dehydration assay described above with the modification that the K2CO₃ concentration used covered a range of 0.15 M to 1 M. The data obtained are presented in FIG. 2, which indicates that, after heating, the carbonic anhydrase of SEQ ID NO: 24 was markedly more active than the similarly treated wild type control, when assayed in the presence of increased levels of K2CO₃.

### Example 9

### C-terminal Fusions Providing Increased Carbonic Anhydrase Stability

This example illustrates construction of a truncation library of carbonic anhydrase variants having varying lengths of the 21 amino acid C-terminal fusion of SEQ ID NO: 24 (“G05”) to determine the minimum length of this additional C-terminal extension (or “tail”) that confers improved stability. The C-terminal fusion appears to have occurred due to a frame shift caused by a single nucleotide deletion at position 633 of the wild-type polynucleotide sequence of SEQ ID NO: 1.

In order to determine whether shorter C-terminal fusion sequences conferred equal or improved stability, a library of twenty-one carbonic anhydrase variants were constructed with C-terminal extension lengths increasing in one amino acid residue increments from all 0 extra amino acids (also referred to as “G05-21” with “-21” indicating 21 extra amino acids truncated) up to a 20 amino acid extension (referred to as “G05-1” with “-1” indicating 1 extra amino acid truncated).

The twenty-one truncation library variants were obtained by introducing two stop codons (TGA, TAA) after the codon for the extension amino acid residue where truncation was desired during the PCR amplification reaction of SEQ ID NO: 23 (the polynucleotide sequence encoding the polypeptide of SEQ ID NO: 24). A silent mutation, A219A (GCC→GCG), also was introduced into SEQ ID NO: 23 in order to destroy an internal SfiI site. PCR products were digested with SfiI, gel purified, ligated into pCK110900, and ligations were transformed into E. coli W3110 fhuA. Preparation of polynucleotide sequences having the desired 21 different extensions were confirmed by sequencing. The polynucleotide and translated amino acid sequences of the full-length variant polypeptides in the truncation library are provided in the sequence listing as SEQ ID NOs: 59-100. The amino acid sequences of the C-terminal extensions alone are also shown in TABLE 3 and provided in the sequence listing as SEQ ID NOs: 101-118.

The truncation library variants were heat challenged at 75° C. in 150 mM K2CO₃, pH 10.9 to determine the minimum tail length that confers equal or improved stability when compared to the parent variant of SEQ ID NO: 24 (also referred to as “G05”). The truncation library variants (“G05-1” through “G05-21”) were assayed in 150 mM K2CO₃, pH 10.9, 400 μM phenolphthalein.

As indicated by the results shown in FIG. 4, a C-terminal extension as short as the 6 amino acids of SEQ ID NO: 88 (G05-15) can still provide an increase in thermostability relative to wild-type that is the equivalent to that provided by the 21 amino acid extension of SEQ ID NO: 24. An exception was SEQ ID NO: 82 (G05-12) which had a 9 amino acid C-terminal extension but exhibited slightly lower thermostability than SEQ ID NO: 24 under the conditions tested. Several of the variants having truncated C-terminal extensions showed increased stability relative to SEQ ID NO: 24, including SEQ ID NO: 60 (G05-1), SEQ ID NO: 66 (G05-4), SEQ ID NO: 72 (G05-7), and SEQ ID NO: 84 (G05-13). Furthermore, as shown by a comparison of SEQ ID NO: 98 (G05-20) to SEQ ID NO: 100 (G05-21), the use of a C-terminal extension of only 1 additional lysine amino acid was sufficient to improve thermostability. These results suggest that the length of the C-terminal extension alone is not the only factor contributing to the thermal stability, and the amino acid composition of the tail as a whole or a particular ending residue may also significantly contribute.

### Example 10

### Secretion of Recombinant Carbonic Anhydrase by Transformed Bacillus megaterium

Secretion of a recombinant (engineered) carbonic anhydrase polypeptide can facilitate large-scale production of the enzyme for use in industrial carbon capture and sequestration processes. This example illustrates construction of a signal peptide construct of the recombinant carbonic anhydrase polypeptide corresponding to SEQ ID NO: 24 and secretion of this carbonic anhydrase from the Bacillus species B. megaterium. The polynucleotide of SEQ ID NO: 23 (which encodes the engineered carbonic anhydrase of SEQ ID NO: 24) was modified by PCR to remove the starting methionine and add SpeI and NgoMVI restriction sites. This modified construct was cloned into the SpeI and NgoMVI restriction sites into the E. coli-B. megaterium shuttle vector pMM1522 (MoBiTec, Goettingen, Germany) The pMM1522 shuttle vector had been modified by the inclusion of one of three different signal peptide sequences capable of providing protein secretion via the SEC pathway in Bacillus megaterium. The N-terminal modification of SEQ ID NO: 24 to provide a SpeI site allowed the corresponding 5′-modified gene of SEQ ID NO: 23 to be cloned in-frame with the signal peptide. The resulting secreted polypeptide would include all of the amino acids at positions 2 to 235 of SEQ ID NO: 24 and at its N-terminus an X-Thr-Ser amino acid sequence (X being the +1 amino acid from the native protein of the corresponding signal peptide sequence) instead of the Met at position 1. The three different signal peptide sequences tested were NprM (extracellular protease signal); YngK (a signal for a homologue of a B. subtilis defense protein); and PenG (the signal for penicillin G acylase), having the sequences shown in Table 4 below.

TABLE 4
Signal peptide sequences evaluated
for CA secretion from B. megaterium

| | |
|---|---|
| NprM | MKKKKQALKVLLSVGILSSSFAFAHTSSA |
| | (SEQ ID NO: 313) |
| YngK | MYIKKCIGSILFLLLFCSSALPAKA (SEQ ID NO: 314) |
| PenG | MKTKWLISVIILFVFIFPQNLVFA (SEQ ID NO: 315) |

Expression of the signal sequence and modified SEQ ID NO: 23 were under the control of a xylA promoter and a xylR repressor protein. The vector also contained the oriU origin of replication, the repU gene, and a tetracycline gene for selection in Bacillus. The vector sequence was confirmed prior to transformation into B. megaterium using standard techniques.

Following transformation, cultures were grown up in shake flask under four different media conditions as follows. Single colonies were inoculated into shake flasks containing 50 mL of either LB (Luria Broth), 2xYT, TB (Terrific Broth), or A5, 0.3% glucose media, and 10 mg/mL Tet media were induced with 0.5% xylose and allowed to grow overnight at 37° C. As controls, the gene of SEQ ID NO: 23 without any signal peptide sequence and empty vector were also transformed and cultured. Culture supernatants and cell lysates were assayed for carbonic anhydrase activity as described in the 1^{st} tier screening assay of Example 11 below.

The media supernatants from the cultured B. megaterium transformants containing the engineered carbonic anhydrase gene of SEQ ID NO: 23 and the YngK, NprM, or PenG signal peptides all exhibited their highest relative carbonic anhydrase activities when cultured in LB media, with relative activities in supernatants of approximately 14, 8, and 2.5, respectively. In contrast, the LB culture of B. megatarium transformants containing the same gene constructs but without the signal peptide exhibited approximately 0.5 relative carbonic anhydrase activity. Empty vector exhibited no activity. For each of the YngK, NprM, and PenG, signal peptide constructs, lower relative activities were observed for cultures grown in 2yt, TB, and A5 media as follows: YngK/2yt˜8.5; NprM/2yt˜6.5; NprM/A5˜5.5; YngK/TB˜5; NprM/TB˜4.8; YngK/A5˜2; PenG/2yt˜1.7; PenG/TB˜1.5. The supernatant of the control gene construct without signal peptide exhibited a relative activity of <1 except for in TB media where a relative activity of ˜1.8 was observed.

SDS-PAGE analysis also was carried out on the concentrated media supernatant from the B. megaterium transformants containing the engineered carbonic anhydrase gene of SEQ ID NO: 23 and the NprM signal peptide grown in LB and A5 media. A strong band observed at ˜28 kD under both media conditions was confirmed by N-terminal amino-acid sequencing to be the expected recombinant carbonic anhydrase polypeptide corresponding to SEQ ID NO: 24. SDS-PAGE analysis of cell lysate showed a band migrating at a slightly higher MW that was confirmed as a polypeptide corresponding to SEQ ID NO: 24. This observation suggests that a portion of the recombinant carbonic anhydrase polypeptide was retained inside the cell.

### Example 11

### Preparation of Recombinant Carbonic Anhydrase Polypeptides with Additional Amino Acid Substitutions Resulting in Improved Enzyme Properties Based on SEQ ID No: 24

A library of engineered polynucleotides was designed and constructed based on the sequence encoding the recombinant carbonic anhydrase polypeptide of SEQ ID NO: 24. The libraries were designed to include all 19 amino acid substitutions at each of the residues corresponding to position 2 through position 235 of SEQ ID NO: 24.

The library was constructed using automated parallel splicing-by-overlap extension PCR, where specific mutations are introduced at various positions along the protein using mutagenic primers based on degenerate primer set: TWG, NNT, and TGG. The library was sub-divided into three pools to facilitate sequencing and screening. The three sub-libraries were cloned into the SpeI-NgoMIV restriction sites of the E. coli-B. megaterium shuttle vector pMM1522 (MoBiTec, Goettingen, Germany) in translational fusion to the NprM signal peptide. The signal peptide sequence NprM, starts with the initial ATG codon and ends with the codon encoding the +1 amino acid of the native NprM protein. The signal peptide sequence was cloned into the shuttle vector between the BsrG1 and SpeI sites. The cloned vectors were then transformed into E. coli, and subsequently transformed into B. megaterium. Colonies of each of the three sub-libraries in B. megatarium were picked, sub-cultured, and harvested as follows:

Picking: Nunc 96-well shallow flat bottom plates were filled with 150 μL/well of picking media (LB, 10 μg/mL tet). Library and control clones were picked into master plates according to the plate layout (streptomyces pins, dip 5 times, 350 mL agar volume setting, 48 pin inoculation). Plates were grown overnight (18-20 hours) in Kuhner shaker (200 rpm, 37° C., and 85% relative humidity).

Subculture: Master plates were visually inspected to ensure even growth in each well. Overnight growth was determined by taking OD of a 1:10 dilution of one of the master plates. Costar 96-well deep plates were filled with 390 μL/well of subculture media (A5 complete media, 10 μg/mL tet). 10 μL., of overnight subculture growth was transferred into deep well plates and allowed to continue growing for 2 hours in Kuhner shaker (250 rpm, 37° C., 85% humidity) to about 0.2-0.3 O.D. Deep well plate cultures were induced by addition of 40 μL/well of 11% xylose and 5 mM ZnSO₄ in sterile water. Final concentration of xylose in each well was about 1% and 0.5 mM of ZnSO₄. After induction wells were allowed to grow overnight (18-24 hours) in Kuhner shaker (250 rpm, 30° C., 85% humidity). Following overnight growth, 70 μL/well of 50% glycerol was added to plates which were heat sealed, shaken (2 min on Micromix shaker), and stored in −80° C. freezer bins.

Harvest: Plates were centrifuged at 4000 rpm and 4° C. for 25 minutes. Supernatant (170-200 μL) was transferred to wells of a new 96 Costar Plate. Plates were stored at 4° C.

Two tiers of library screening were carried out on the harvested supernatant.

First-tier high throughput screening: Supernatant samples containing engineered carbonic anhydrase secreted by B. megaterium were challenged by incubation for 15 minutes at 55° C. in 1.25 M 2-amino-2-methyl-1-propanol (AMP) and assayed using the 1^{st} tier Endpoint Assay as follows.

Assay mix (50 mL for 275 reactions) was prepared by combining and mixing: 100 μL of 100 mM Thymol Blue (final conc.=200 μM), 5 mL of 1 M HEPES pH 7.0 (final conc.=100 mM), 6 mL of 10.427 M AMP (final conc.=1.25 M), and 38.9 mL of ddH₂O. Bubble 100% CO₂ (g) into solution for ˜1 hour. Transfer 180 μl., of Assay mix into each well of a polystyrene square well plate. Add 20 μL of supernatant into assay mix plates. Incubate assay plate(s) in incubator (55° C.) on shaker for 15 min. Remove plates from incubator and allow to incubate at Room Temperature for at least 30 minutes. A color distinction between the positive (blue) and negative (yellow) wells should become apparent. Briefly spin plates at 4° C. 4000 rpm. Read plate(s) at 600 nm and 440 nm using an M2 plateReader and SoftPro Max software with the following parameter settings: Endpoint; Monitor two wavelengths (Lm1=600 nm; Lm2=440 nm); Mix 3 sec Before Reading; Pathcheck on.

Second-tier screening: Supernatant samples showing >1.2-fold improvement over positive control in the 1^{st} tier screen were challenged by 30-60 minutes room temperature incubation in 1.5 M AMP (pH 9.7) and assayed for improved activity (e.g., increased rate) using a 2^{nd} tier Kinetic Assay as follows.

Assay mix (for 200 μL reaction) was prepared by combining: 0.8 μL of 0.1 M phenolphthalein (final conc.=400 μM), 28.8 μL of 10.427M AMP, pH 9.7 (final conc.=1.5 M), and 170.4 μL of ddH₂O. Transfer plate(s) containing assay mix to CO₂ chamber (20% CO₂ blend with 80% compress air) and place on a shaker with gentle shaking. The pH indicator dye turns from deep pink to clear upon equilibration of solution with CO₂.

Set up SoftPro Max software on M2 plateReader outside of CO₂ chamber with the following parameter settings: Absorbance mode; Kinetic read; 550 nm wavelength; 30 min duration, 37 sec intervals; 3 seconds of shaking before the 1^{st} read as well as in between subsequence reads.

Transfer CO₂ equilibrated plates from CO₂ chamber to plate reader. Inspect plate(s) and remove any bubbles before reading on the plate reader. Click READ to start SoftPro Max. Relative carbonic anhydrase activity is determined using SLOPE value from SoftPro Max or by calculating slope using exported absorbance versus time values.

Table 5 below lists the sequence identifiers, sequence features, and relative carbonic anhydrase activities (based on 2^{nd} tier screening results) of engineered carbonic anhydrase secreted by B. megatarium that showed improved activity relative to the positive control of the polypeptide of SEQ ID NO: 120 following thermal and solvent challenge.

TABLE 5

| SEQ ID | Amino acid substitutions | Activity FIOP |
|---|---|---|
| NO: | (as compared to SEQ ID | (as compared to |
| (nt/aa) | NO: 120) | SEQ ID NO: 120) |
| 119/120 | — | 1.00 |
| 121/122 | A191P; | 1.15 |
| 123/124 | N147A; | 1.34 |
| 125/126 | P16V; | 1.16 |
| 127/128 | A57V; | 1.39 |
| 129/130 | H194G; | 1.06 |
| 131/132 | A127R; | 1.53 |
| 133/134 | A26S; | 1.82 |
| 135/136 | E105W; | 1.29 |
| 137/138 | H214M; | 2.32 |
| 139/140 | T46L; | 1.82 |
| 141/142 | E3W; | 1.67 |
| 143/144 | A33G; | 1.68 |
| 145/146 | H194E; | 1.29 |
| 147/148 | E3A; P66G; | 1.24 |
| 149/150 | N147H; | 1.09 |
| 151/152 | P27L; | 1.06 |
| 153/154 | K212R; | 1.4 |
| 155/156 | Q2N; N11P; | 1.25 |
| 157/158 | C149S; | 1.14 |
| 159/160 | T161N; | 1.85 |
| 161/162 | E44A; A156T; | 1.18 |
| 163/164 | E44Q; | 1.62 |
| 165/166 | P27E; | 1.18 |
| 167/168 | H214E; | 1.8 |
| 169/170 | D36A; | 1.13 |
| 171/172 | H214W; | 1.02 |
| 173/174 | E3A; | 1.26 |
| 175/176 | V6M; | 1.05 |
| 177/178 | H214C; | 1.29 |
| 179/180 | P22K; | 1.6 |
| 181/182 | Q2P; T46S; | 1.68 |
| 183/184 | P31D; | 1.12 |
| 185/186 | K104Q; | 1.07 |
| 187/188 | E105T; | 1.31 |
| 189/190 | A138S; | 1.75 |
| 191/192 | E3L; | 2.45 |
| 193/194 | E14F; | 1.89 |
| 195/196 | V6Q; | 1.34 |
| 197/198 | D36H; | 1.53 |
| 199/200 | S7P; | 1.74 |
| 201/202 | Q2A; S10V; T46V; | 1.22 |
| 203/204 | E8A; | 1.24 |
| 205/206 | S40C; | 1.43 |
| 207/208 | Q137G; | 1.65 |
| 209/210 | G165K; | 1.28 |
| 211/212 | T46D; | 2.21 |
| 213/214 | H214D; | 2.05 |
| 215/216 | Q2H; | 1.47 |
| 217/218 | S10W; P37H; | 1.32 |
| 219/220 | A127E; H214K; | 1.54 |
| 221/222 | E23G; | 1.17 |
| 223/224 | H194A; | 1.3 |
| 225/226 | E23S; | 1.27 |
| 227/228 | P31Q; | 1.46 |
| 229/230 | N203I | 1.57 |
| 231/232 | E44P; | 1.62 |
| 233/234 | P31C; | 1.7 |
| 235/236 | E8Q; | 1.05 |
| 237/238 | A127W; | 1.28 |
| 239/240 | K142Q; | 1.21 |
| 241/242 | P22I; | 1.48 |
| 243/244 | I98V; | 1.03 |
| 245/246 | I98K; | 1.32 |
| 247/248 | M136Q; | 1.04 |
| 249/250 | F139M; | 1.56 |
| 251/252 | F139V; | 1.17 |
| 253/254 | V204T; | 1.33 |
| 255/256 | V204Q; | 1.21 |
| 257/258 | R226P; | 1.22 |
| 259/260 | T222G; | 1.1 |
| 261/262 | R226D; | 1.31 |
| 263/264 | L235T; | 1.61 |
| 265/266 | L235V; | 1.36 |
| 267/268 | L235S; | 1.36 |
| 269/270 | I225M; | 1.06 |
| 271/272 | M230A; | 1.18 |
| 273/274 | A216S; | 1.04 |
| 275/276 | T220G; | 1.29 |
| 277/278 | K215A; | 1.24 |
| 279/280 | T222E; | 1.31 |
| 281/282 | I225L; | 1.57 |
| 283/284 | T220N; | 1.21 |
| 285/286 | R226G; | 1.9 |
| 287/288 | G231D; | 1.26 |
| 289/290 | L233Q; | 1.17 |
| 291/292 | K217G; | 1.1 |
| 293/294 | I225C; | 1.17 |
| 295/296 | I221G; | 1.1 |
| 297/298 | I223T; | 1.23 |
| 299/300 | T220D; | 1.56 |
| 301/302 | I225G; | 1.01 |

As shown by the results summarized in Table 5, the following amino acid substitutions in the core structure (i.e., positions 2 to 214) of the polypeptide of SEQ ID NO: 120 improved carbonic anhydrase activity, tolerance to prolonged exposure to the solvent AMP, and tolerance to high temperature (55° C.) for 15 minutes: Q2AHNP, E3ALW, V6MQ, S7P, E8AQ, S10VW; N11P, E14F, P16V, P221K, E23GS, A26S, P27EL, P31CDQ, A33G, D36AH, P37H, S40C, E44APQ, T46DLSV, A57V, I98 KV, K104Q, E105TW, A127ERW; M136Q, Q137G, A138S; F139MV, K142Q, N147AH, C149S, A156T; T161N, G165K, A191P; H194AEG, N203I, V204GQT, K212R, and H214CDEMWK.

As shown by the results summarized in Table 5, the following amino acid substitutions in the 21 amino acid C-terminal extension (or “tail”) structure (i.e., positions 215 to 235) of the polypeptide of SEQ ID NO: 120 improved carbonic anhydrase activity, tolerance to prolonged exposure to the solvent AMP, and tolerance to high temperature (55° C.) for 15 minutes: K215A, A216S, K217G, T220DGN, I221GT, T222EG, I225CGLM, R226DGP, M230A, G231D, L233Q, and L235STV. The amino acid sequences of the C-terminal extensions alone are also shown in Table 3 and provided in the sequence listing as SEQ ID NOs: 316-338.

As shown by the results summarized in Table 6, the following nucleotide substitutions (relative to SEQ ID NO: 119) that do not encode amino acid substitutions (i.e., “silent mutations”) also appear to result in increased activity likely due to increased expression and/or secretion into the supernatant: g48t; c165t; t160a; a217t; a300g; a333t; t453g; a537g; c612t; and t618g.

TABLE 6

| | Nucleotide differences | Activity FIOP |
|---|---|---|
| SEQ ID NO: | (as compared to SEQ | (as compared to |
| (nt) | ID NO: 119) | SEQ ID NO: 120) |
| 303 | a537g; | 1.02 |
| 304 | t160a; | 1.2 |
| 305 | a300g; | 1.42 |
| 306 | g48t; | 1.11 |
| 307 | c165t; | 1.04 |
| 308 | a333t; | 1.29 |
| 309 | a217t; | 1.1 |
| 310 | t453g; | 1.22 |
| 311 | t618g; | 1.54 |
| 312 | c612t; | 1.04 |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A recombinant carbonic anhydrase polypeptide having an improved enzyme property relative to a reference polypeptide of SEQ ID NO:2, wherein said polypeptide comprises an amino acid sequence having at least 80% identity to SEQ ID NO:2 and one or more of the following amino acid substitutions at the position corresponding to the indicated position of SEQ ID NO: 2:
residue at position 2 is alanine, histidine, asparagine, or proline;
residue at position 3 is tryptophan;
residue at position 7 is proline;
residue at position 8 is alanine, or glutamine;
residue at position 10 is valine, or tryptophan;
residue at position 11 is proline;
residue at position 14 is phenylalanine;
residue at position 16 is valine;
residue at position 22 is isoleucine, or lysine;
residue at position 23 is lysine, or serine;
residue at position 26 is serine;
residue at position 27 is glutamic acid, or leucine;
residue at position 31 is cysteine, or aspartic acid;
residue at position 33 is glycine;
residue at position 36 is alanine;
residue at position 37 is histidine;
residue at position 40 is cysteine;
residue at position 46 is aspartic acid, leucine, serine, or valine;
residue at position 56 is cysteine, or histidine;
residue at position 57 is valine;
residue at position 58 is valine;
residue at position 87 is threonine;
residue at position 90 is lysine;
residue at position 95 is glutamine;
residue at position 98 is lysine;
residue at position 105 is threonine, or tryptophan;
residue at position 127 is glutamic acid, or arginine;
residue at position 131 is asparagine;
residue at position 136 is glutamine;
residue at position 137 is glycine;
residue at position 142 is glutamine;
residue at position 147 is alanine, or histidine;
residue at position 149 is serine;
residue at position 156 is threonine;
residue at position 161 is asparagine;
residue at position 165 is asparagine, or lysine;
residue at position 191 is proline;
residue at position 194 is alanine, glutamic acid, or glycine;
residue at position 195 is methionine;
residue at position 203 is isoleucine;
residue at position 212 is glycine;
residue at position 213 is leucine;
residue at position 214 is cysteine, aspartic acid, glutamic acid, histidine, lysine, methionine, or tryptophan.

2. The recombinant carbonic anhydrase polypeptide of claim 1, wherein the amino acid sequence further comprises one or more of the following amino acid substitutions at the position corresponding to the indicated position of SEQ ID NO: 2:
residue at position 3 is alanine, leucine, or tryptophan;
residue at position 6 is methionine, or glutamine;
residue at position 7 is proline, or serine;
residue at position 23 is glycine, lysine, or serine;
residue at position 31 is cysteine, aspartic acid, or glutamine;
residue at position 36 is alanine, or histidine;
residue at position 40 is cysteine, or valine;
residue at position 44 is alanine, proline, or glutamine;
residue at position 98 is lysine, or valine;
residue at position 104 is glutamine;
residue at position 105 is threonine, or tryptophan;
residue at position 122 is isoleucine;
residue at position 127 is glutamic acid, arginine, or tryptophan;
residue at position 138 is serine;
residue at position 139 is methionine, or valine;
residue at position 204 is glycine, glutamine, or threonine;
residue at position 208 is valine;
residue at position 212 is arginine, glycine, or lysine.

3. The recombinant carbonic anhydrase polypeptide of claim 1, wherein the amino acid sequence further comprises one or more of the following amino acid substitutions at the position corresponding to the indicated position of SEQ ID NO: 2:
residue at position 7 is proline, or serine;
residue at position 212 is arginine, glycine, or lysine.

4. The recombinant carbonic anhydrase polypeptide of claim 1, wherein the amino acid sequence comprises at least two of the following amino acid substitutions at the position corresponding to the indicated position of SEQ ID NO: 2:
residue at position 7 is proline, or serine;
residue at position 212 is arginine, glycine, or lysine;
residue at position 213 is leucine;
residue at position 214 is cysteine, aspartic acid, glutamic acid, histidine, lysine, methionine, or tryptophan.

5. The recombinant carbonic anhydrase polypeptide of claim 1, wherein the amino acid sequence comprises the following amino acid substitutions at the position corresponding to the indicated position of SEQ ID NO: 2:
residue at position 7 is serine;
residue at position 212 is lysine;
residue at position 213 is leucine; and
residue at position 214 is histidine.

6. The recombinant carbonic anhydrase polypeptide of claim 1, wherein the amino acid sequence further comprises one or more of the following amino acid substitutions at the position corresponding to the indicated position of SEQ ID NO: 2:
Q2A; Q2H; Q2N; Q2P; E3A; E3L; E3W; V6M; V6Q; D7P; D7S; E8A; E8Q; S10V; S10W; N11P; E14F; P16V; P22I; P22K; E23G; E23K; E23S; A26S; P27E; P27L; P31C; P31D; P31Q; A33G; D36A; D36H; P37H; S40C; S40V; E44A; E44P; E44Q; T46D; T46L; T46S; T46V; M56C; M56H; A57V; S58V; P66G; I87T; E90K; E95K; E95Q; I98K; I98V; K104Q; E105T; E105W; V122I; A127E; A127R; A127W; D131N; M136Q; Q137G; A138S; F139M; F139V; K142Q; N147A; N147H; C149S; A156T; T161N; G165K; G165N; A191P; H194A; H194E; H194G; T195M; N203I; V204Q; V204T; E208V; E212G; E212K; E212R; T213L; S214C; S214D; S214E; S214H; S214K; S214M; S214W.

7. The recombinant carbonic anhydrase polypeptide of claim 1, wherein the amino acid sequence further comprises a carboxy terminal fusion of any one of the polypeptides of SEQ ID NOs: 101-118, 316-338, KAK, KA, or the single amino acid K.

8. The recombinant carbonic anhydrase polypeptide of claim 7, wherein the amino acid sequence further comprises a carboxy terminal fusion of a polypeptide of SEQ ID NO: 101.

9. The recombinant carbonic anhydrase polypeptide of claim 8, wherein the amino acid sequence comprises one or more of the following amino acid substitutions at the position corresponding to the indicated position of a polypeptide comprising SEQ ID NO: 2 and a carboxy terminal fusion of a polypeptide of SEQ ID NO: 101:
Q2A; Q2H; Q2N; Q2P; E3A; E3L; E3W; V6M; V6Q; D7P; D7S; E8A; E8Q; S10V; S10W; N11P; E14F; P16V; P22I; P22K; E23G; E23K; E23S; A26S; P27E; P27L; P31C; P31D; P31Q; A33G; D36A; D36H; P37H; 540C; 540V; E44A; E44P; E44Q; T46D; T46L; T46S; T46V; M56C; M56H; A57V; S58V; P66G; I87T; E90K; E95K; E95Q; I98K; I98V; K104Q; E105T; E105W; V122I; A127E; A127R; A127W; D131N; M136Q; Q137G; A138S; F139M; F139V; K142Q; N147A; N147H; C149S; A156T; T161N; G165K; G165N; A191P; H194A; H194E; H194G; T195M; N203I; V204Q; V204T; E208V; E212G; E212K; E212R; T213L; S214C; S214D; S214E; S214H; S214K; S214M; S214W; K215A; A216S; K217G; T220D; T220G; T220N; I221G; T222E; T222G; I223T; I225C; I225G; I225L; I225M; R226D; R226G; R226P; M230A; G231D; L233Q; L235S; L235T; L235V.

10. A recombinant carbonic anhydrase polypeptide having an improved enzyme property relative to a reference polypeptide of SEQ ID NO:120, wherein said polypeptide comprises an amino acid sequence having at least 80% identity to SEQ ID NO:120 and one or more of the following amino acid substitutions at the position corresponding to the indicated position of SEQ ID NO: 2:
Q2A; Q2H; Q2N; Q2P; E3A; E3L; E3W; V6M; V6Q; S7D; S7P; E8A; E8Q; 510V; 510W; N11P; E14F; P16V; P22I; P22K; E23G; E23K; E23S; A26S; P27E; P27L; P31C; P31D; P31Q; A33G; D36A; D36H; P37H; S40C; S40V; E44A; E44P; E44Q; T46D; T46L; T46S; T46V; M56C; M56H; A57V; S58V; P66G; I87T; E90K; E95K; E95Q; I98K; I98V; K104Q; E105T; E105W; V122I; A127E; A127R; A127W; D131N; M136Q; Q137G; A138S; F139M; F139V; K142Q; N147A; N147H; C149S; A156T; T161N; G165K; G165N; A191P; H194A; H194E; H194G; T195M; N203I; V204Q; V204T; E208V; K212E; K212G; K212R; T213L; H214C; H214D; H214E; H214S; H214K; H214M; H214W; K215A; A216S; K217G; T220D; T220G; T220N; I221G; T222E; T222G; I223T; I225C; I225G; I225L; I225M; R226D; R226G; R226P; M230A; G231D; L233Q; L235S; L235T; L235V.

11. The recombinant carbonic anhydrase polypeptide of claim 10, wherein the improved enzyme property is at least 1.2-fold increased rate of hydrating carbon dioxide to bicarbonate in the presence of about 1.5 M AMP and the one or more amino acid substitutions at the position corresponding to the indicated position of SEQ ID NO: 2 are selected from the following:
Q2A; Q2H; Q2N; Q2P; E3A; E3L; E3W; V6Q; S7P; E8A; 510V; 510W; N11P; E14F; P22I; P22K; E23S; A26S; P31C; P31Q; A33G; D36H; P37H; 540C; E44P; E44Q; T46D; T46L; T46S; T46V; A57V; P66G; I98K; E105T; E105W; A127E; A127R; A127W; Q137G; A138S; F139M; K142Q; N147A; T161N; G165K; H194A; H194E; N203I; V204Q; V204T; K212R; H214C; H214D; H214E; H214K; H214M; K215A; T220D; T220G; T220N; T222E; I223T; I225L; R226D; R226G; R226P; G231D; L235S; L235T; and L235V.

12. The recombinant carbonic anhydrase polypeptide of claim 10, wherein the improved enzyme property is at least 1.2-fold increased rate of hydrating carbon dioxide to bicarbonate in the presence of about 1.5 M AMP and the one or more amino acid substitutions at the position corresponding to the indicated position of SEQ ID NO: 2 are selected from the following:
Q2P; E3L; E3W; S7P; E14F; P22K; A26S; P31C; A33G; D36H; E44P; E44Q; T46D; T46L; T46S; A127E; A127R; Q137G; A138S; F139M; T161N; N203I; H214D; H214E; H214K; H214M; T220D; 1225L; R226G; and L235T.

13. The recombinant carbonic anhydrase polypeptide of claim 1, wherein said improved enzyme property is increased rate of hydrating carbon dioxide to bicarbonate.

14. The recombinant carbonic anhydrase polypeptide of claim 13, wherein said rate is increased at least 1.2-times, that of the reference polypeptide having the amino acid sequence of SEQ ID NO: 2.

15. The recombinant carbonic anhydrase polypeptide of claim 13, wherein said rate is measured in the presence of from about 0.1 M K2CO₃.

16. The recombinant carbonic anhydrase polypeptide of claim 13, wherein said rate is determined after heating the recombinant carbonic anhydrase polypeptide and the reference polypeptide at a temperature of from about 50° C. to 100° C. for a period of time of about 5 minutes to about 180 minutes.

17. The recombinant carbonic anhydrase polypeptide of claim 13, wherein said rate is determined in the presence of from about 0.1 M K2CO₃ to about 0.5 M K2CO₃ after heating the recombinant carbonic anhydrase polypeptide and the reference polypeptide at a temperature within the range of from about 50° C. to 100° C. for a period of time within the range of from about 5 minutes to about 180 minutes, and said rate is determined.

18. The recombinant carbonic anhydrase polypeptide of claim 13, wherein said rate is determined in the presence of a co-solvent selected from the group consisting of: monoethanolamine (MEA), methyldiethanolamine (MDEA), 2-aminomethylpropanolamine (AMP), 2-(2-aminoethylamino)ethanol (AEE), triethanolamine, 2-amino-2-hydroxymethyl-1,3-propanediol (Tris), piperazine, dimethyl ether of polyethylene glycol (PEG DME), ammonia, and mixtures thereof.

19. The recombinant carbonic anhydrase polypeptide of claim 13, wherein said rate is determined in the presence of from about 0.5 M AMP to about 3.0 M AMP.

20. The recombinant carbonic anhydrase polypeptide of claim 13 wherein said rate is determined at a pH of from about pH 8 to about pH 12.

21. The recombinant carbonic anhydrase polypeptide of claim 1 which comprises an amino acid sequence selected from the group consisting of SEQ ID NOS: 4, 6, 10, 12, 14, 16, 20, 22, 24, 28, 36, 38, 44, 50, 56, 58, 60, 62, 64, 66, 68, 70, 72, 74, 76, 78, 80, 82, 84, 86, 88, 90, 92, 94, 96, 98, 100, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302.

22. A polynucleotide encoding a recombinant carbonic anhydrase polypeptide of claim 1.

23. The polynucleotide of claim 22 which comprises a nucleotide sequence selected from the group consisting of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 304, 305, 306, 307, 308, 309, 310, 311, and 312.

24. An expression vector comprising the polynucleotide of claim 22 operably linked to control sequences capable of directing expression of the encoded polypeptide in a host cell.

25. (canceled)

26. (canceled)

27. (canceled)

28. (canceled)

29. (canceled)

30. A host cell comprising the expression vector of claim 24.

31. (canceled)

32. (canceled)

33. (canceled)

34. The host cell of claim 30, wherein codons comprising the expression vector have been optimized for expression in the host cell.

35. A method of producing a recombinant carbonic anhydrase polypeptide comprising:
(a) transforming a host cell with an expression vector polynucleotide encoding a recombinant carbonic anhydrase polypeptide of claim 1;
(b) culturing said transformed host cell under conditions whereby said recombinant carbonic anhydrase polypeptide is produced by said host cell; and
(c) recovering said recombinant carbonic anhydrase polypeptide from said host cells.

36. (canceled)

37. A composition comprising the recombinant carbonic anhydrase polypeptide of claim 1 and a solution comprising a solvent selected from the group consisting of: monoethanolamine (MEA), methyldiethanolamine (MDEA), 2-aminomethylpropanolamine (AMP), piperazine, ammonia, and mixtures thereof.

38. A method for removing carbon dioxide from a gas stream comprising the step of contacting the gas stream with a solution comprising a recombinant carbonic anhydrase polypeptide of claim 1, whereby carbon dioxide from the gas stream is dissolved in the solution and converted to hydrated carbon dioxide.

39. The method of claim 38, wherein the solution is aqueous.

40. The method of claim 38, wherein the solution is an aqueous co-solvent system.

41. The method of claim 38, wherein the aqueous-solvent system comprises an organic solvent selected from monoethanolamine, methyldiethanolamine, and 2-aminomethylpropanolamine.

42. The method of claim 38, wherein the recombinant carbonic anhydrase polypeptide is immobilized on a surface.

43. The method of claim 38, wherein the method further comprises the step of isolating the solution comprising hydrated carbon dioxide and contacting the isolated solution with hydrogen ions and a recombinant carbonic anhydrase polypeptide of claim 1, thereby converting the hydrated carbon dioxide to carbon dioxide gas and water.


SEQUENCE LISTING (338 sequences)

SEQ ID NO: 1 - DNA, 642 bp, Artificial Sequence
  codon optimized M. thermophila carbonic anhydrase gene
atgcaggaaataaccgtggatgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 2 - protein, 214 aa, Methanosarcina thermophila
MQEITVDEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 3 - DNA, 705 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 4 - protein, 235 aa, Artificial Sequence
  variant of M. thermophila carbonic anhydrase
MQEITVDEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 5 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggctgtagtcataggagaggtgacaataggtgccaacgttatggtttcccccatggcagtcattaggagtgacgaaggaatgccaattttcgtaggagacagaagcaatgtccaggatggagttgtgcttcatgcccttgagacaataaatgaaaaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 6 - protein, 214 aa, Artificial Sequence
  variant of M. thermophila carbonic anhydrase
MQEITVDEFSNIRENPVTPWNPEPSAPVIDPTAYIDPQAVVIGEVTIGANVMVSPMAVIRSDEGMPIFVGDRSNVQDGVVLHALETINEKGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 7 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggctgtagtcataggagaggtgacaataggtgccaacgttatggtttccccctgcgcagtcattaggagtgacgaaggaatgccaattttcgtaggagacagaagcaatgtccaggatggcgttgtacttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 8 - protein, 214 aa, Artificial Sequence
  variant of M. thermophila carbonic anhydrase
MQEITVDEFSNIRENPVTPWNPEPSAPVIDPTAYIDPQAVVIGEVTIGANVMVSPCAVIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 9 - DNA, 642 bp, Artificial Sequence
  variant of M. thermophila carbonic anhydrase
atgcaggaaataaccgtggatgagttttcgaacatcagagagaatccggtaacaccatggaatcccgaaccatcagcacctgtgattgatcccacagcctatattgacccccaggcttcagtcataggagaggtgacaataggtgccaacgttatggtttccccccatgcatccattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 10 - protein, 214 aa, Artificial Sequence
  variant of M. thermophila carbonic anhydrase
MQEITVDEFSNIRENPVTPWNPEPSAPVIDPTAYIDPQASVIGEVTIGANVMVSPHASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 11 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggctgtagtcataggagaggtgacaataggtgccaacgttatggtttcccccatggcagtcattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 12 - protein, 214 aa, Artificial Sequence
  variant of M. thermophila carbonic anhydrase
MQEITVDEFSNIRENPVTPWNPEPSAPVIDPTAYIDPQAVVIGEVTIGANVMVSPMAVIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 13 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggcttcagtcataggagaggtgacaataggtgccaacgttatggtttccccccatgcagtcattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 14 - protein, 214 aa, Artificial Sequence
  variant of M. thermophila carbonic anhydrase
MQEITVDEFSNIRENPVTPWNPEPSAPVIDPTAYIDPQASVIGEVTIGANVMVSPHAVIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 15 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggcttcagtcataggagaggtgacaataggtgccaacgttatggtttccccccatgcatccattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 16 - protein, 214 aa, Artificial Sequence; identical to SEQ ID NO: 10
  variant of M. thermophila carbonic anhydrase

SEQ ID NO: 17 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggctgtagtcataggagaggtgacaataggtgccaacgttatggtttccccctgcgcagtcattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 18 - protein, 214 aa, Artificial Sequence; identical to SEQ ID NO: 8
  variant of M. thermophila carbonic anhydrase

SEQ ID NO: 19 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggcttcagtcataggagaggtgacaataggtgccaacgttatggtttccccccatgcatccattaggagtgacgaaggaatgccaattttcgtaggagacagaagcaatgtccaggatggagttgtgcttcatgcccttgagacaacaaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccacactaacgaagccgtagtatatgtaaacgtccaccttgccgagggttataaagaaacttca

SEQ ID NO: 20 - protein, 214 aa, Artificial Sequence
  variant of M. thermophila carbonic anhydrase
MQEITVDEFSNIRENPVTPWNPEPSAPVIDPTAYIDPQASVIGEVTIGANVMVSPHASIRSDEGMPIFVGDRSNVQDGVVLHALETTNEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 21 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggcttcagtcataggagaggtgacaataggtgccaacgttatggtttccccccatgcatccattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaaggaacttca

SEQ ID NO: 22 - protein, 214 aa, Artificial Sequence
  variant of M. thermophila carbonic anhydrase
MQEITVDEFSNIRENPVTPWNPEPSAPVIDPTAYIDPQASVIGEVTIGANVMVSPHASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKGTS

SEQ ID NO: 23 - DNA, 705 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 24 - protein, 235 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 25 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgcgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 26 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHMNEAVVYVNVHLAEGYKETS

SEQ ID NO: 27 - DNA, 642 bp, Artificial Sequence
  variant of M. thermophila carbonic anhydrase
atgcaggaaataaccgtgagtgagttttcgaatatcagagagaatccggtaacaccatggaatcccaaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacaacaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 28 - protein, 214 aa, Artificial Sequence
  variant of M. thermophila carbonic anhydrase
MQEITVSEFSNIRENPVTPWNPKPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDNRYIPAGMVVTSQAEADKLPEVTDDYAYSHTNEAVVYVNVHLAEGYKETS

SEQ ID NO: 29 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 30 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 31 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctataaaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 32 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIKDNIVEVDGKEYAVYIGNNVSLAHQSQVHGPAAVGDNTFIGMQAFVFKSKVGNNCVLEPRSAAIGVTIPDGRYIPAGMVVTSQAEADKLPEVTDDYAYSHMNEAVVYVNVHLAEGYKETS

SEQ ID NO: 33 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacccgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 34 - protein, 214 aa, Artificial Sequence; identical to SEQ ID NO: 26
  variant of M. thermophila carbonic anhydrase

SEQ ID NO: 35 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctataaaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaggaaacttca

SEQ ID NO: 36 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIKDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHMNEAVVYVNVHLAEGYKETS

SEQ ID NO: 37 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 38 - protein, 214 aa, Artificial Sequence; identical to SEQ ID NO: 26
  variant of M. thermophila carbonic anhydrase

SEQ ID NO: 39 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgtgtacttgagccgagatcggcagcaatcggtgtaaccattcctgacaacaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 40 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDNTFIGMQAFVFKSKVGNNCVLEPRSAAIGVTIPDNRYIPAGMVVTSQAEADKLPEVTDDYAYSHMNEAVVYVNVHLAEGYKETS

SEQ ID NO: 41 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatacaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacaacaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 42 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIQDNIVEVDGKEYAVYIGNNVSLAHQSQVHGPAAVGDDTFIGMQAFVFKSKVGNNCVLEPRSAAIGVTIPDNRYIPAGMVVTSQAEADKLPEVTDDYAYSHMNEAVVYVNVHLAEGYKETS

SEQ ID NO: 43 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacgataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctataaaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgcgtacttgagccgagatcggcagcaatcggtgtaaccattcctgacaacaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 44 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIKDNIVEVDGKEYAVYIGNNVSLAHQSQVHGPAAVGDNTFIGMQAFVFKSKVGNNCVLEPRSAAIGVTIPDNRYIPAGMVVTSQAEADKLPEVTDDYAYSHMNEAVVYVNVHLAEGYKETS

SEQ ID NO: 45 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatacaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgtgtacttgagccgagatcggcagcaatcggtgtaaccattcctgacaacaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 46 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIQDNIVEVDGKEYAVYIGNNVSLAHQSQVHGPAAVGDNTFIGMQAFVFKSKVGNNCVLEPRSAAIGVTIPDNRYIPAGMVVTSQAEADKLPEVTDDYAYSHMNEAVVYVNVHLAEGYKETS

SEQ ID NO: 47 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 48 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDNTFIGMQAFVFKSKVGNNCVLEPRSAAIGVTIPDGRYIPAGMVVTSQAEADKLPEVTDDYAYSHMNEAVVYVNVHLAEGYKETS

SEQ ID NO: 49 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgtgtacttgagccgagatcggcagcaatcggtgtaaccattcctgacaacaggtatatcccggcaggcatggttgtcacttcgcaggccgaagccgataagctgccagaggtcaccgatgattacgcatacagccatactaacgaagccgtagtatatgtaaacgtccaccttgccgtgggttataaagaaacttca

SEQ ID NO: 50 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDNTFIGMQAFVFKSKVGNNCVLEPRSAAIGVTIPDNRYIPAGMVVTSQAEADKLPEVTDDYAYSHTNEAVVYVNVHLAVGYKETS

SEQ ID NO: 51 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatacaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 52 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIQDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHMNEAVVYVNVHLAEGYKETS

SEQ ID NO: 53 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacaggagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 54 - protein, 214 aa, Artificial Sequence; identical to SEQ ID NO: 48
  variant of M. thermophila carbonic anhydrase

SEQ ID NO: 55 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctataaaagacaacattgttgaagttgatggcaaggaatacgcagtctatataggaaataatgtttcgcttgctcatcaatcccaggttcatggtccggcggcagtaggcgataatacatttatcggtatgcaggctttcgttttcaagtcaaaggtagggaacaactgtgtacttgagccgagatcggcagcaatcggtgtaaccattcctgacaacaggtatatcccggcaggcatggttgtcacttcgcaggccgaagccgataagctgccagaggtcaccgatgattacgcatacagccatatgaacgaagccgtagtatatgtaaacgtccaccttgccgagggttataaagaaacttca

SEQ ID NO: 56 - protein, 214 aa, Artificial Sequence; identical to SEQ ID NO: 44
  variant of M. thermophila carbonic anhydrase

SEQ ID NO: 57 - DNA, 642 bp, Artificial Sequence
  variant of M. thermophila carbonic anhydrase
atgcaggaaataaccgtggatgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccagattcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataaagaaacttca

SEQ ID NO: 58 - protein, 214 aa, Artificial Sequence
  variant of M. thermophila carbonic anhydrase
MQEITVDEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQIHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 59 - DNA, 705 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaagagcagatgggcaaacttgactga

SEQ ID NO: 60 - protein, 234 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLD

SEQ ID NO: 61 - DNA, 699 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaagagcagatgggcaaactt

SEQ ID NO: 62 - protein, 233 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKL

SEQ ID NO: 63 - DNA, 696 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaagagcagatgggcaaa

SEQ ID NO: 64 - protein, 232 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGK

SEQ ID NO: 65 - DNA, 693 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaagagcagatgggc

SEQ ID NO: 66 - protein, 231 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMG

SEQ ID NO: 67 - DNA, 690 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaagagcagatg

SEQ ID NO: 68 - protein, 230 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQM

SEQ ID NO: 69 - DNA, 687 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaagagcag

SEQ ID NO: 70 - protein, 229 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQ

SEQ ID NO: 71 - DNA, 684 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaagag

SEQ ID NO: 72 - protein, 228 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREE

SEQ ID NO: 73 - DNA, 681 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagggaa

SEQ ID NO: 74 - protein, 227 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIRE

SEQ ID NO: 75 - DNA, 678 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccattagg

SEQ ID NO: 76 - protein, 226 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIR

SEQ ID NO: 77 - DNA, 675 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcaccatt

SEQ ID NO: 78 - protein, 225 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITI

SEQ ID NO: 79 - DNA, 672 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatcacc

SEQ ID NO: 80 - protein, 224 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITIT

SEQ ID NO: 81 - DNA, 669 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcaccatc

SEQ ID NO: 82 - protein, 223 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITI

SEQ ID NO: 83 - DNA, 666 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatcacc

SEQ ID NO: 84 - protein, 222 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATIT

SEQ ID NO: 85 - DNA, 663 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgaccatc

SEQ ID NO: 86 - protein, 221 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATI

SEQ ID NO: 87 - DNA, 660 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcgacc

SEQ ID NO: 88 - protein, 220 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLAT

SEQ ID NO: 89 - DNA, 657 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggcg

SEQ ID NO: 90 - protein, 219 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLA

SEQ ID NO: 91 - DNA, 654 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactg

SEQ ID NO: 92 - protein, 218 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKL

SEQ ID NO: 93 - DNA, 651 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaa

SEQ ID NO: 94 - protein, 217 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAK

SEQ ID NO: 95 - DNA, 648 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggcc

SEQ ID NO: 96 - protein, 216 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKA

SEQ ID NO: 97 - DNA, 645 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataag

SEQ ID NO: 98 - protein, 215 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHK

SEQ ID NO: 99 - DNA, 642 bp, Artificial Sequence
  variant of M. thermophila carbonic anhydrase
atgcaggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcat

SEQ ID NO: 100 - protein, 214 aa, Artificial Sequence
  variant of M. thermophila carbonic anhydrase
MQEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLH

SEQ ID NO: 101 - protein, 21 aa, Artificial Sequence
  C-terminal fusion sequence
KAKLATITITIREEQMGKLDL

SEQ ID NO: 102 - protein, 20 aa, Artificial Sequence
  C-terminal fusion sequence
KAKLATITITIREEQMGKLD

SEQ ID NO: 103 - protein, 19 aa, Artificial Sequence
  C-terminal fusion sequence
KAKLATITITIREEQMGKL

SEQ ID NO: 104 - protein, 18 aa, Artificial Sequence
  C-terminal fusion sequence
KAKLATITITIREEQMGK

SEQ ID NO: 105 - protein, 17 aa, Artificial Sequence
  C-terminal fusion sequence
KAKLATITITIREEQMG

SEQ ID NO: 106 - protein, 16 aa, Artificial Sequence
  C-terminal fusion sequence
KAKLATITITIREEQM

SEQ ID NO: 107 - protein, 15 aa, Artificial Sequence
  C-terminal fusion sequence
KAKLATITITIREEQ

SEQ ID NO: 108 - protein, 14 aa, Artificial Sequence
  C-terminal fusion sequence
KAKLATITITIREE

SEQ ID NO: 109 - protein, 13 aa, Artificial Sequence
  C-terminal fusion sequence
KAKLATITITIRE

SEQ ID NO: 110 - protein, 12 aa, Artificial Sequence
  C-terminal fusion sequence
KAKLATITITIR

SEQ ID NO: 111 - protein, 11 aa, Artificial Sequence
  C-terminal fusion sequence
KAKLATITITI

SEQ ID NO: 112 - protein, 10 aa, Artificial Sequence
  C-terminal fusion sequence
KAKLATITIT

SEQ ID NO: 113 - protein, 9 aa, Artificial Sequence
  C-terminal fusion sequence
KAKLATITI

SEQ ID NO: 114 - protein, 8 aa, Artificial Sequence
  C-terminal fusion sequence
KAKLATIT

SEQ ID NO: 115 - protein, 7 aa, Artificial Sequence
  C-terminal fusion sequence
KAKLATI

SEQ ID NO: 116 - protein, 6 aa, Artificial Sequence
  C-terminal fusion sequence
KAKLAT

SEQ ID NO: 117 - protein, 5 aa, Artificial Sequence
  C-terminal fusion sequence
KAKLA

SEQ ID NO: 118 - protein, 4 aa, Artificial Sequence
  C-terminal fusion sequence
KAKL

SEQ ID NO: 119 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 120 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 121 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacccttacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 122 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYPYSHTNEAVVYVNVHLAEGYKKLHKAKLATITITIREEQMGKLDL

SEQ ID NO: 123 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtaggggctaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 124 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGANCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 125 - DNA, 702 bp, Artificial Sequence
  Variant of M. thermophila carbonic anhydrase
caggaaataaccgtgagtgagttttcgaatatcagagagaatgttgtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 126 - protein, 234 aa, Artificial Sequence
  Variant of M. thermophila carbonic anhydrase
QEITVSEFSNIRENVVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 127 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggtttctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 128 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMVSIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 129 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagcggtactaacgaagccgtagtatatgtaaacgtccaccttgccgagggttataagaaacttcataaggccaaactggccaccatcaccatcaccattagggaagagcagatgggcaagcttgacctg

SEQ ID NO: 130 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSGTNEAVVYVNVHLAEGYKKLHKAKLATITITIREEQMGKLDL

SEQ ID NO: 131 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcgcgtgtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 132 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPARVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 133 - DNA, 702 bp, Artificial Sequence
  Variant of M. thermophila carbonic anhydrase
caggaaataaccgtgagtgagttttcgaatatcagagagaatccggtaacaccatggaatcccgaaccatcatctcctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 134 - protein, 234 aa, Artificial Sequence
  Variant of M. thermophila carbonic anhydrase
QEITVSEFSNIRENPVTPWNPEPSSPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 135 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaagtggtacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 136 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKWYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 137 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttatgaaggccaaactggccaccatcaccatcaccattagggaagagcagatgggcaagcttgacctg

SEQ ID NO: 138 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLMKAKLATITITIREEQMGKLDL

SEQ ID NO: 139 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgctgataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 140 - protein, 234 aa, Artificial Sequence
  Variant of M. thermophila carbonic anhydrase
QEITVSEFSNIRENPVTPWNPEPSAPVIDPTAYIDPQASVIGEVLIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 141 - DNA, 702 bp, Artificial Sequence
  Variant of M. thermophila carbonic anhydrase
cagtggataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 142 - protein, 234 aa, Artificial Sequence
  Variant of M. thermophila carbonic anhydrase
QWITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 143 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacaggttatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 144 - protein, 234 aa, Artificial Sequence
  Variant of M. thermophila carbonic anhydrase
QEITVSEFSNIRENPVTPWNPEPSAPVIDPTGYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 145 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagcgagactaacgaagccgtagtatatgtaaacgtccaccttgccgagggttataagaaacttcataaggccaaactggccaccatcaccatcaccattagggaagagcagatgggcaagcttgacctg

SEQ ID NO: 146 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSETNEAVVYVNVHLAEGYKKLHKAKLATITITIREEQMGKLDL

SEQ ID NO: 147 - DNA, 702 bp, Artificial Sequence
  Variant of M. thermophila carbonic anhydrase
caggctataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgggtattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 148 - protein, 234 aa, Artificial Sequence
  Variant of M. thermophila carbonic anhydrase
QAITVSEFSNIRENPVTPWNPEPSAPVIDPTAYIDPQASVIGEVTIGANVMVSPMASIRSDEGMGIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 149 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggcataactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 150 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGHNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 151 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacttgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 152 - protein, 234 aa, Artificial Sequence
  Variant of M. thermophila carbonic anhydrase
QEITVSEFSNIRENPVTPWNPEPSALVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 153 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagcgtcttcataaggccaaactggccaccatcaccatcaccattagggaagagcagatgggcaagcttgacctg

SEQ ID NO: 154 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKRLHKAKLATITITIREEQMGKLDL

SEQ ID NO: 155 - DNA, 702 bp, Artificial Sequence
  Variant of M. thermophila carbonic anhydrase
aatgaaataaccgtgagtgagttttcgcctatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 156 - protein, 234 aa, Artificial Sequence
  Variant of M. thermophila carbonic anhydrase
NEITVSEFSPIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 157 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactctgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 158 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNSVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 159 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaatattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 160 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVNIPDGRYIPAGMVVTSQAEADKLPEVTDDYAYSHTNEAVVYVNVHLAEGYKKLHKAKLATITITIREEQMGKLDL

SEQ ID NO: 161 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagctgtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcgacagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 162 - protein, 234 aa, Artificial Sequence
  Variant of M. thermophila carbonic anhydrase
QEITVSEFSNIRENPVTPWNPEPSAPVIDPTAYIDPQASVIGAVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSTAIGVTIPDGRYIPAGMVVTSQAEADKLPEVTDDYAYSHTNEAVVYVNVHLAEGYKKLHKAKLATITITIREEQMGKLDL

SEQ ID NO: 163 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggacaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 164 - protein, 234 aa, Artificial Sequence
  Variant of M. thermophila carbonic anhydrase
QEITVSEFSNIRENPVTPWNPEPSAPVIDPTAYIDPQASVIGQVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 165 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcagaggtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 166 - protein, 234 aa, Artificial Sequence
  Variant of M. thermophila carbonic anhydrase
QEITVSEFSNIRENPVTPWNPEPSAEVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 167 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttgagaaggccaaactggccaccatcaccatcaccattagggaagagcagatgggcaagcttgacctg

SEQ ID NO: 168 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLEKAKLATITITIREEQMGKLDL

SEQ ID NO: 169 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgctccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 170 - protein, 234 aa, Artificial Sequence
  Variant of M. thermophila carbonic anhydrase
QEITVSEFSNIRENPVTPWNPEPSAPVIDPTAYIAPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 171 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaactttggaaggccaaactggccaccatcaccatcaccattagggaagagcagatgggcaagcttgacctg

SEQ ID NO: 172 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLWKAKLATITITIREEQMGKLDL

SEQ ID NO: 173 - DNA, 702 bp, Artificial Sequence
  Variant of M. thermophila carbonic anhydrase
caggctataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 174 - protein, 234 aa, Artificial Sequence
  Variant of M. thermophila carbonic anhydrase
QAITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 175 - DNA, 702 bp, Artificial Sequence
  Variant of M. thermophila carbonic anhydrase
caggaaataaccatgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 176 - protein, 234 aa, Artificial Sequence
  Variant of M. thermophila carbonic anhydrase
QEITMSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 177 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaactttgtaaggccaaactggccaccatcaccatcaccattagggaagagcagatgggcaagcttgacctg

SEQ ID NO: 178 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLCKAKLATITITIREEQMGKLDL

SEQ ID NO: 179 - DNA, 702 bp, Artificial Sequence
  Variant of M. thermophila carbonic anhydrase
caggaaataaccgtgagtgagttttcgaatatcagagagaatccggtaacaccatggaataaggaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 180 - protein, 234 aa, Artificial Sequence
  Variant of M. thermophila carbonic anhydrase
QEITVSEFSNIRENPVTPWNKEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 181 - DNA, 702 bp, Artificial Sequence
  Variant of M. thermophila carbonic anhydrase
cctgaaataaccgtgagtgagttttcgaatatcagagagaatccggtaacaccatggaatcccgaaccatcagcacctgtgattgatcccacagcctatattgacccccaggcttcagtcataggagaggtgagtataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 182 - protein, 234 aa, Artificial Sequence
  Variant of M. thermophila carbonic anhydrase
PEITVSEFSNIRENPVTPWNPEPSAPVIDPTAYIDPQASVIGEVSIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 183 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatgatacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 184 - protein, 234 aa, Artificial Sequence
  Variant of M. thermophila carbonic anhydrase
QEITVSEFSNIRENPVTPWNPEPSAPVIDDTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 185 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggccaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 186 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGQEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 187 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaagacttacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 188 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKTYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 189 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcagtctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 190 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQSFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 191 - DNA, 702 bp, Artificial Sequence
  Variant of M. thermophila carbonic anhydrase
cagcttataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 192 - protein, 234 aa, Artificial Sequence
  Variant of M. thermophila carbonic anhydrase
QLITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 193 - DNA, 702 bp, Artificial Sequence
  Variant of M. thermophila carbonic anhydrase
caggaaataaccgtgagtgagttttcgaatatcagatttaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 194 - protein, 234 aa, Artificial Sequence
  Variant of M. thermophila carbonic anhydrase
QEITVSEFSNIRFNPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 195 - DNA, 702 bp, Artificial Sequence
  Variant of M. thermophila carbonic anhydrase
caggaaataacccagagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 196 - protein, 234 aa, Artificial Sequence
  Variant of M. thermophila carbonic anhydrase
QEITQSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 197 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattcatccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 198 - protein, 234 aa, Artificial Sequence
  Variant of M. thermophila carbonic anhydrase
QEITVSEFSNIRENPVTPWNPEPSAPVIDPTAYIHPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 199 - DNA, 702 bp, Artificial Sequence
  Variant of M. thermophila carbonic anhydrase
caggaaataaccgtgcctgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 200 - protein, 234 aa, Artificial Sequence
  Variant of M. thermophila carbonic anhydrase
QEITVPEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 201 - DNA, 702 bp, Artificial Sequence
  Variant of M. thermophila carbonic anhydrase
gctgaaataaccgtgagtgagtttgttaatatcagagagaatccggtaacaccatggaatcccgaaccatcagcacctgtgattgatcccacagcctatattgacccccaggcttcagtcataggagaggtggtgataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 202 - protein, 234 aa, Artificial Sequence
  Variant of M. thermophila carbonic anhydrase
AEITVSEFVNIRENPVTPWNPEPSAPVIDPTAYIDPQASVIGEVVIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 203 - DNA, 702 bp, Artificial Sequence
  Variant of M. thermophila carbonic anhydrase
caggaaataaccgtgagtgctttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 204 - protein, 234 aa, Artificial Sequence
  Variant of M. thermophila carbonic anhydrase
QEITVSAFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 205 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttgtgtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 206 - protein, 234 aa, Artificial Sequence
  Variant of M. thermophila carbonic anhydrase
QEITVSEFSNIRENPVTPWNPEPSAPVIDPTAYIDPQACVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 207 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgggggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 208 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMGAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 209 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacaagaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 210 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDKRYIPAGMVVTSQAEADKLPEVTDDYAYSHTNEAVVYVNVHLAEGYKKLHKAKLATITITIREEQMGKLDL

SEQ ID NO: 211 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtggatataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 212 - protein, 234 aa, Artificial Sequence
  Variant of M. thermophila carbonic anhydrase
QEITVSEFSNIRENPVTPWNPEPSAPVIDPTAYIDPQASVIGEVDIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 213 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttgataaggccaaactggccaccatcaccatcaccattagggaagagcagatgggcaagcttgacctg

SEQ ID NO: 214 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLDKAKLATITITIREEQMGKLDL

SEQ ID NO: 215 - DNA, 702 bp, Artificial Sequence
  Variant of M. thermophila carbonic anhydrase
catgaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 216 - protein, 234 aa, Artificial Sequence
  Variant of M. thermophila carbonic anhydrase
HEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 217 - DNA, 702 bp, Artificial Sequence
  Variant of M. thermophila carbonic anhydrase
caggaaataaccgtgagtgagttttggaatatcagagagaatccggtaacaccatggaatcccgaaccatcagcacctgtgattgatcccacagcctatattgaccatcaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 218 - protein, 234 aa, Artificial Sequence
  Variant of M. thermophila carbonic anhydrase
QEITVSEFWNIRENPVTPWNPEPSAPVIDPTAYIDHQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 219 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggaagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttaagaaggccaaactggccaccatcaccatcaccattagggaagagcagatgggcaagcttgacctg

SEQ ID NO: 220 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAEVGDDTFIGMQAFVFKSKVGNNCVLEPRSAAIGVTIPDGRYIPAGMVVTSQAEADKLPEVTDDYAYSHTNEAVVYVNVHLAEGYKKLKKAKLATITITIREEQMGKLDL

SEQ ID NO: 221 - DNA, 702 bp, Artificial Sequence
  Variant of M. thermophila carbonic anhydrase
caggaaataaccgtgagtgagttttcgaatatcagagagaatccggtaacaccatggaatcccggtccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 222 - protein, 234 aa, Artificial Sequence
  Variant of M. thermophila carbonic anhydrase
QEITVSEFSNIRENPVTPWNPGPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 223 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagcgctactaacgaagccgtagtatatgtaaacgtccaccttgccgagggttataagaaacttcataaggccaaactggccaccatcaccatcaccattagggaagagcagatgggcaagcttgacctg

SEQ ID NO: 224 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSATNEAVVYVNVHLAEGYKKLHKAKLATITITIREEQMGKLDL

SEQ ID NO: 225 - DNA, 702 bp, Artificial Sequence
  Variant of M. thermophila carbonic anhydrase
caggaaataaccgtgagtgagttttcgaatatcagagagaatccggtaacaccatggaatccctctccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 226 - protein, 234 aa, Artificial Sequence
  Variant of M. thermophila carbonic anhydrase
QEITVSEFSNIRENPVTPWNPSPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 227 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcagacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 228 - protein, 234 aa, Artificial Sequence
  Variant of M. thermophila carbonic anhydrase
QEITVSEFSNIRENPVTPWNPEPSAPVIDQTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 229 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaatcgtccaccttgccgagggttataagaaacttcataaggccaaactggccaccatcaccatcaccattagggaagagcagatgggcaagcttgacctg

SEQ ID NO: 230 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVIVHLAEGYKKLHKAKLATITITIREEQMGKLDL

SEQ ID NO: 231 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggacctgtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 232 - protein, 234 aa, Artificial Sequence
  Variant of M. thermophila carbonic anhydrase
QEITVSEFSNIRENPVTPWNPEPSAPVIDPTAYIDPQASVIGPVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 233 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgattgtacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 234 - protein, 234 aa, Artificial Sequence
  Variant of M. thermophila carbonic anhydrase
QEITVSEFSNIRENPVTPWNPEPSAPVIDCTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 235 - DNA, 702 bp, Artificial Sequence
  Variant of M. thermophila carbonic anhydrase
caggaaataaccgtgagtcagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 236 - protein, 234 aa, Artificial Sequence
  Variant of M. thermophila carbonic anhydrase
QEITVSQFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 237 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcgtgggtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 238 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAWVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 239 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttccagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 240 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFQSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 241 - DNA, 702 bp, Artificial Sequence
  Variant of M. thermophila carbonic anhydrase
caggaaataaccgtgagtgagttttcgaatatcagagagaatccggtaacaccatggaatattgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 242 - protein, 234 aa, Artificial Sequence
  Variant of M. thermophila carbonic anhydrase
QEITVSEFSNIRENPVTPWNIEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 243 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacgttgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 244 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNVVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 245 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacaaggttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 246 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNKVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 247 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtcagcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 248 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGQQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 249 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctatggttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 250 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAMVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 251 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctgttgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 252 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAVVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDL

SEQ ID NO: 253 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacactcaccttgccgagggttataagaaacttcataaggccaaactggccaccatcaccatcaccattagggaagagcagatgggcaagcttgacctg

SEQ ID NO: 254 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNTHLAEGYKKLHKAKLATITITIREEQMGKLDL

SEQ ID NO: 255 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaaccagcaccttgccgagggttataagaaacttcataaggccaaactggccaccatcaccatcaccattagggaagagcagatgggcaagcttgacctg

SEQ ID NO: 256 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNQHLAEGYKKLHKAKLATITITIREEQMGKLDL

SEQ ID NO: 257 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattcctgaagagcagatgggcaagcttgacctg

SEQ ID NO: 258 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIPEEQMGKLDL

SEQ ID NO: 259 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcggtatcaccattagggaagagcagatgggcaagcttgacctg

SEQ ID NO: 260 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATIGITIREEQMGKLDL

SEQ ID NO: 261 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattgatgaagagcagatgggcaagcttgacctg

SEQ ID NO: 262 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIDEEQMGKLDL

SEQ ID NO: 263 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacact

SEQ ID NO: 264 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDT

SEQ ID NO: 265 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacgtt

SEQ ID NO: 266 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDV

SEQ ID NO: 267 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgactct

SEQ ID NO: 268 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKLDS

SEQ ID NO: 269 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccatgagggaagagcagatgggcaagcttgacctg

SEQ ID NO: 270 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITMREEQMGKLDL

SEQ ID NO: 271 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcaggctggcaagcttgacctg

SEQ ID NO: 272 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQAGKLDL

SEQ ID NO: 273 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataagtctaaactggccaccatcaccatcaccattagggaagagcagatgggcaagcttgacctg

SEQ ID NO: 274 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKSKLATITITIREEQMGKLDL

SEQ ID NO: 275 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccggtatcaccatcaccattagggaagagcagatgggcaagcttgacctg

SEQ ID NO: 276 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLAGITITIREEQMGKLDL

SEQ ID NO: 277 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcatgctgccaaactggccaccatcaccatcaccattagggaagagcagatgggcaagcttgacctg

SEQ ID NO: 278 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHAAKLATITITIREEQMGKLDL

SEQ ID NO: 279 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcgagatcaccattagggaagagcagatgggcaagcttgacctg

SEQ ID NO: 280 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATIEITIREEQMGKLDL

SEQ ID NO: 281 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccctgagggaagagcagatgggcaagcttgacctg

SEQ ID NO: 282 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITLREEQMGKLDL

SEQ ID NO: 283 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaatatcaccatcaccattagggaagagcagatgggcaagcttgacctg

SEQ ID NO: 284 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLANITITIREEQMGKLDL

SEQ ID NO: 285 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattggtgaagagcagatgggcaagcttgacctg

SEQ ID NO: 286 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIGEEQMGKLDL

SEQ ID NO: 287 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatggataagcttgacctg

SEQ ID NO: 288 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMDKLDL

SEQ ID NO: 289 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcaggacctg

SEQ ID NO: 290 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITIREEQMGKQDL

SEQ ID NO: 291 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccggtctggccaccatcaccatcaccattagggaagagcagatgggcaagcttgacctg

SEQ ID NO: 292 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAGLATITITIREEQMGKLDL

SEQ ID NO: 293 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcacctgtagggaagagcagatgggcaagcttgacctg

SEQ ID NO: 294 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITCREEQMGKLDL

SEQ ID NO: 295 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccggtaccatcaccattagggaagagcagatgggcaagcttgacctg

SEQ ID NO: 296 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATGTITIREEQMGKLDL

SEQ ID NO: 297 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccactaccattagggaagagcagatgggcaagcttgacctg

SEQ ID NO: 298 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITTTIREEQMGKLDL

SEQ ID NO: 299 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccgatatcaccatcaccattagggaagagcagatgggcaagcttgacctg

SEQ ID NO: 300 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLADITITIREEQMGKLDL

SEQ ID NO: 301 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccggtagggaagagcagatgggcaagcttgacctg

SEQ ID NO: 302 - protein, 234 aa, Artificial Sequence
  Variant of M. thermophila carbonic anhydrase
QEITVSEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKKLHKAKLATITITGREEQMGKLDL

SEQ ID NO: 303 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaggccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 304 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggttagtcccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 305 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaggttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 306 - DNA, 702 bp, Artificial Sequence
  Variant of M. thermophila carbonic anhydrase
caggaaataaccgtgagtgagttttcgaatatcagagagaatcctgtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 307 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccctatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 308 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggtaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 309 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagatctaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 310 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtactggagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccaccttgccgagggttataagaaacttcataaggccaaactggccaccatcaccatcaccattagggaagagcagatgggcaagcttgacctg

SEQ ID NO: 311 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgtccacctggccgagggttataagaaacttcataaggccaaactggccaccatcaccatcaccattagggaagagcagatgggcaagcttgacctg

SEQ ID NO: 312 - DNA, 702 bp, Artificial Sequence
  Variant of M. thermophila carbonic anhydrase
caggaaataaccgtgagtgagttttcgaatatcagagagaatccggtaacaccatggaatcccgaaccatcagcacctgtgattgatcccacagcctatattgacccccaggcttcagtcataggagaggtgacaataggtgccaacgttatggtttcccccatggcatctattaggagtgacgaaggaatgccaattttcgtaggagacagaagcaatgtccaggatggagttgtgcttcatgcccttgagacaataaatgaagaaggcgaacctatagaagacaacattgttgaagttgatggcaaggaatacgcagtctatataggaaataatgtttcgcttgctcatcaatcccaggttcatggtccggcggcagtaggcgatgatacatttatcggtatgcaggctttcgttttcaagtcaaaggtagggaacaactgtgtacttgagccgagatcggcagcaatcggtgtaaccattcctgacggcaggtatatcccggcaggcatggttgtcacttcgcaggccgaagccgataagctgccagaggtcaccgatgattacgcatacagccatactaacgaagccgtagtatatgtaaacgttcaccttgccgagggttataagaaacttcataaggccaaactggccaccatcaccatcaccattagggaagagcagatgggcaagcttgacctg

SEQ ID NO: 313 - protein, 29 aa, Artificial sequence
  nprm signal peptide
MKKKKQALKVLLSVGILSSSFAFAHTSSA

SEQ ID NO: 314 - protein, 25 aa, Artificial sequence
  YngK signal peptide
MYIKKCIGSILFLLLFCSSALPAKA

SEQ ID NO: 315 - protein, 24 aa, Artificial sequence
  PenG signal peptide
MKTKWLISVIILFVFIFPQNLVFA

SEQ ID NO: 316 - protein, 21 aa, Artificial sequence
  C-terminal fusion sequence
KAKLATITITIPEEQMGKLDL

SEQ ID NO: 317 - protein, 21 aa, Artificial sequence
  C-terminal fusion sequence
KAKLATIGITIREEQMGKLDL

SEQ ID NO: 318 - protein, 21 aa, Artificial sequence
  C-terminal fusion sequence
KAKLATITITIDEEQMGKLDL

SEQ ID NO: 319 - protein, 21 aa, Artificial sequence
  C-terminal fusion sequence
KAKLATITITIREEQMGKLDT

SEQ ID NO: 320 - protein, 21 aa, Artificial sequence
  C-terminal fusion sequence
KAKLATITITIREEQMGKLDV

SEQ ID NO: 321 - protein, 21 aa, Artificial sequence
  C-terminal fusion sequence
KAKLATITITIREEQMGKLDS

SEQ ID NO: 322 - protein, 21 aa, Artificial sequence
  C-terminal fusion sequence
KAKLATITITMREEQMGKLDL

SEQ ID NO: 323 - protein, 21 aa, Artificial sequence
  C-terminal fusion sequence
KAKLATITITIREEQAGKLDL

SEQ ID NO: 324 - protein, 21 aa, Artificial sequence
  C-terminal fusion sequence
KSKLATITITIREEQMGKLDL

SEQ ID NO: 325 - protein, 21 aa, Artificial sequence
  C-terminal fusion sequence
KAKLAGITITIREEQMGKLDL

SEQ ID NO: 326 - protein, 21 aa, Artificial sequence
  C-terminal fusion sequence
AAKLATITITIREEQMGKLDL

SEQ ID NO: 327 - protein, 21 aa, Artificial sequence
  C-terminal fusion sequence
KAKLATIEITIREEQMGKLDL

SEQ ID NO: 328 - protein, 21 aa, Artificial sequence
  C-terminal fusion sequence
KAKLATITITLREEQMGKLDL

SEQ ID NO: 329 - protein, 21 aa, Artificial sequence
  C-terminal fusion sequence
KAKLANITITIREEQMGKLDL

SEQ ID NO: 330 - protein, 21 aa, Artificial sequence
  C-terminal fusion sequence
KAKLATITITIGEEQMGKLDL

SEQ ID NO: 331 - protein, 21 aa, Artificial sequence
  C-terminal fusion sequence
KAKLATITITIREEQMDKLDL

SEQ ID NO: 332 - protein, 21 aa, Artificial sequence
  C-terminal fusion sequence
KAKLATITITIREEQMGKQDL

SEQ ID NO: 333 - protein, 21 aa, Artificial sequence
  C-terminal fusion sequence
KAGLATITITIREEQMGKLDL

SEQ ID NO: 334 - protein, 21 aa, Artificial sequence
  C-terminal fusion sequence
KAKLATITITCREEQMGKLDL

SEQ ID NO: 335 - protein, 21 aa, Artificial sequence
  C-terminal fusion sequence
KAKLATGTITIREEQMGKLDL

SEQ ID NO: 336 - protein, 21 aa, Artificial sequence
  C-terminal fusion sequence
KAKLATITTTIREEQMGKLDL

SEQ ID NO: 337 - protein, 21 aa, Artificial sequence
  C-terminal fusion sequence
KAKLADITITIREEQMGKLDL

SEQ ID NO: 338 - protein, 21 aa, Artificial sequence
  C-terminal fusion sequence
KAKLATITITGREEQMGKLDL